SEQUENCE LISTING (96 sequences)

SEQ ID NO: 1 - DNA, 4594 bp, Sorghum bicolor
  source 1..4594: mol_type = genomic DNA
atggcggcggcgccgaggcccaagtcgccgcctgcctcgccggacccctgcggccgccaccgcctccagctcgccgtcgacgcgctccaccgggagatcagcttcctcgaggtatgtatgtatgtatgtatgtacggatcactcaatactagctaagctagctagctaccacctgcatgccattcttcgattctagctcgcatatatattatgctgcagtgctcgaccgatctttgcagtttgcatcacctacctatcctcttgctgccttgctcagctgcgtttgccattttccttctctctctctctctctctctctctcttatgtgtttcaaaaatgcatatataaacaatcgcattcagaaaatgatatacaaggataaataaagtgtgcgtagtcaacgaagttcaaatttaccagttgctggtggctcctctccatatgggccggccggccgagaactgaaacgctatcagcagtagtagatgacaccgtttaactttgaaagcttaattagtttaatccatcctagctgtgctaatggtcagcagtttaatctctctctgcctctgggcacgtgcggatcgtaccatggatttgcgcgcgtgtcaagcctcaactctgctctttatattccgtttgcctaccatttttttcccacgattccgactcgtctcagagcttggattcttcttctcttctaacctgctgatgatgatattatactacgggcgtgtttagatccaaaatttttttggattttgacattgtagcactttcgtttttatttaacaaacattgttcaatcatggagtaactaggtttaaaagattcgtctcgcgatttacagacaaactgtgtaattagtttttatttttatctatatttaatacttcatacatgtgccgcaagattcgatgtgatggggaatcttgtaaagttttgggtttttgagtgtatctaaacaaggcctacattacttctctcacacacacaatctcaaataaagcactattactactactactaatgatttttttttgtctatctatcgatctggctagggtttaagggatctaggatctagtacatggaagaagatacaagggccggcccacgctttgattattgcctttgtgtcacctgcgtatatcctaaatactactccgatcctatatcctatcatatggcttggaggctttcactacaataatataatactacacccatgtaggcgtcagtgtgtcactataatgctacattagcgctaaaccatagcagctgtacttgcgaaaacatgtttcagatcagcaggctcttcctccttcaaggcttcaactcatcatgaaaaatttcgtccagcccggcctggcctttgcttgccggacaccttctcgttttaatgccttgttattatttatgttatatctgaaataatatatcagcagcacgcatactggacataaagttgcttctcatggtgccacatttccaactactcatccctgtccagcagcctgctcactcatcacttcatcagagctcagctacccagcaataaaatattcccgaaaacagatgttaattgaacactattatttactgcccgatgagcctgcaaaaacacagccctgcattggtgccatgcctacgtgccatctcggtatgtaagagttaattaatcttaactgacaaatttattcaaatactccacaactgatggttctacttcaaactgatcatgacctactaggctaccacaccagcttgtgtgtatgcgaatgcatggcttccttccttgatgcatatagtacctccccccccttacataataaccttaggccttgtttagtttctaaaaatttttaaggttctccgtcacattgaatcttgcggcacatgcatgaagcattatatatagacgaaaataaaaactaattacacaatttgcctgtaaatcgtgagacgaatcttttgaccctagttagtctatgattggacaatatttgccacaaacaaacgaaagtgctacagtagcgaaatccaaaaaatttcgcgaactgaaccaggccttattaatttgttacaagattgtttgtttcttctgttccaacacagcaacgaagcattatatcaaaaaaaaaaacgacgttgcattgatgccaaaatatgctggcgtacggccgtttgcatatggttgcagactgcagtatctcgttctattagaatattggaaagctcggcccccagctggggttttcttcagtaaactcaaactttgaatgtgctgtgtgttaaaaatatcccagacgacaacgcctgaaaagttgacagctgaacacatggcttttacttaccagcagtatacagtagtatttactattaccaatatgatattattatattatatatcctgaaacgctcgatttaatttctttgctgcagggcgaaataagttccattgagggggtccacgctgcctccagatgctgcaaagagtaagctcaacaaatactccattattactactttgtgtacaggggaccttattacttgcagtgttttcacaagaatggctgaaaatgctagtatcagaaaataattcatgaacttaatttctctttaatttaaactgcaaaacatattctcatccaaatgtccatgcatccatccattgttcttcggaatagatactggataactatggttggataactagttttataggtttctatatgagtaaattgaattgcttctctatatattgaccgatgaccatgtggatcgtcggagaaaccaaagatgattctctgttgattgcctctctttaataaatctttgcagggttgatgagtttgttggaagtaatccggatccattcctaacgatgtatgaattttcaggttgtgaatttgtctttaacttagcatgaatgctgcatttttcttattatctctttttaaaaagcagtcagccagagaaaggaagccatgatcaatctcagcagtttctgaagaagttccggtacttcctttgctcctcacaactcttaatattgttggttcttaccattaccagtagcacacgcgcgcatatatagtaatgttattttgttgtacgagaaaaattctaccatcattatctcctaataataatcctaaaatcattgttaggtaggagaatgtacacagaaagataacagtctctgtatatcgtcacaagacataaaactaacaccagccttgtttacttctacccaaaaacctaaaattttttaagattctccgtcacgtcgaatctttagacgcatgcatgtaatattaaatatagacgaaaataaaaactaattacatagttaggtcagaatttacgagacgaatcttttgagtctagttagtccataattagacaataattaccacaaacaaacgaaagtgttacagtgtcgtgaaatctttcccatgaggaactaaacacggcctaaaactgaaatgaatgacacatatatctctccattattactgtctactgtcctattgagaacacaacctgtccattttctgaaaagtatgcaagtgcatattcattcagttcaagctgtatgaagtttgtgatcatctgcgagcctcagtcccaccagcgtacgttctagcagcttcaacggctcgtatcagatctttgctgtttttttcctctgcagcagcctgcacccctccactgtactactgcactacaaactttcagagcaattaccttgtcgtttactactttaaactgccatgccatcaacatcacattatcagccagagcaatcagtttgtcgatcgttgtctgcactaacacttaattgagcatctaaagagctacttttcagaaccgtcacctcattgttactataattgtttcaattattcctcaacatgccgtcagaaaccatcagctactgaatatgtccgtcagtaaaccatgaaatgatgctgctgtttgttgctagctagctagcttgcattgggtttttgggtgaatgattgacaatcgatcacggagcagatgaagttgcttactcttgtgcatgcatgcagagcaaagagctgcctgagctactacctctcgtggatctgctgctgcggcggtggcggtggcggtggcagtggcgggtggtgcccgccgtcgctgcagctcaagaggccagcggcgccgagctgctcctgcgcgccgcggctgaggaagctctgctgctgctgttgctgctgccggtgccgcgtggtgtacgccggcggcgccggcggctgcgggtgctgcgcgccgtgcccgcgctgctcgtgcgactgcacctgcgcctgcccgcgctgctgctcctgccccaggtgcagcgccgcgtgctgctgcgccccgcgctgctgcctgtgcctatgatggccacatgagaaattttgtgtgcatgctactacccggagaaccgtggttttgatgatggatgactggcattttgcagcatgttttttttttgtttagtgtcggcgacacgagtactattgctaacttgctgttctggttcgtgctgcatgctgattgctgaatggtgtcgattcc

SEQ ID NO: 2 - DNA, 1660 bp, synthetic construct
  source 1..1660: mol_type = other DNA
agcgatcgagtagtagtagagggagtagctgccggccgggagcccggagctacagtataggaggaggaggaggaggaggaggaggaggaggaggagtactcttcctagctacagtagctgtgcgttgcgacgagcttctgcattgtgcattgcattgctgcatcatccgatccgtttggtttcccgctcgctccacctcctccttcctcgtcgtctcctgccggctgccactgccaacaaagggcgcgccacgcacggcccggccggtccccccgcactacgcagcagtattccggaaagaaacgaggcgctgctcttgctgctgctgctgctctctcccctccatcattgccgcccgaaacggcaacccgccaccgtcccactgcactgcactgcactgcactcccctccatctccatccatgtgctcgcctgctgcaccacctcgctctcgctcctccactaccgccatttaatccacctcgcctagctcgctgctccttgcacactgctggctggctgcctacctagctactcctagctccctcctccatcattattccggcctgcaagcacacttgctcttgctcgatcattaattagttcgtcgctcgatcgatccctcgccctgcacgcagcaagccccgcccattctgctaccttctgcctgcgcatgcatctccctctcttccttcaattcatcagcaaagccttgccgcgcgccgcgtgcgtaacgtaccagctgctagtagtgcactagtgctacgccgccgccggctctctcgtcgtcgtcgtcgttgacgatcgtcgttcgtccagtatatatatatatatacaccaattaataggatcggataagaagaagaagctactagctagaacagataatggcggcggcgccgaggcccaagtcgccgcctgcctcgccggacccctgcggccgccaccgcctccagctcgccgtcgacgcgctccaccgggagatcagcttcctcgagggcgaaataagttccattgagggggtccacgctgcctccagatgctgcaaagaggttgatgagtttgttggaagtaatccggatccattcctaacgattcagccagagaaaggaagccatgatcaatctcagcagtttctgaagaagttccgagcaaagagctgcctgagctactacctctcgtggatctgctgctgcggcggtggcggtggcggtggcagtggcgggtggtgcccgccgtcgctgcagctcaagaggccagcggcgccgagctgctcctgcgcgccgcggctgaggaagctctgctgctgctgttgctgctgccggtgccgcgtggtgtacgccggcggcgccggcggctgcgggtgctgcgcgccgtgcccgcgctgctcgtgcgactgcacctgcgcctgcccgcgctgctgctcctgccccaggtgcagcgccgcgtgctgctgcgccccgcgctgctgcctgtgcctatgatggccacatgagaaattttgtgtgcatgctactacccggagaaccgtggttttgatgatggatgactggcattttgcagcatgttttttttttgtttagtgtcggcgacacgagtactattgctaacttgctgttctggttcgtgctgcatgctgattgctgaatggtgtcgattcc

SEQ ID NO: 3 - DNA, 597 bp, synthetic construct
  source 1..597: mol_type = other DNA
atggcggcggcgccgaggcccaagtcgccgcctgcctcgccggacccctgcggccgccaccgcctccagctcgccgtcgacgcgctccaccgggagatcagcttcctcgagggcgaaataagttccattgagggggtccacgctgcctccagatgctgcaaagaggttgatgagtttgttggaagtaatccggatccattcctaacgattcagccagagaaaggaagccatgatcaatctcagcagtttctgaagaagttccgagcaaagagctgcctgagctactacctctcgtggatctgctgctgcggcggtggcggtggcggtggcagtggcgggtggtgcccgccgtcgctgcagctcaagaggccagcggcgccgagctgctcctgcgcgccgcggctgaggaagctctgctgctgctgttgctgctgccggtgccgcgtggtgtacgccggcggcgccggcggctgcgggtgctgcgcgccgtgcccgcgctgctcgtgcgactgcacctgcgcctgcccgcgctgctgctcctgccccaggtgcagcgccgcgtgctgctgcgccccgcgctgctgcctgtgcctatga

SEQ ID NO: 4 - protein, 198 aa, Sorghum bicolor
MAAAPRPKSPPASPDPCGRHRLQLAVDALHREISFLEGEISSIEGVHAASRCCKEVDEFVGSNPDPFLTIQPEKGSHDQSQQFLKKFRAKSCLSYYLSWICCCGGGGGGGSGGWCPPSLQLKRPAAPSCSCAPRLRKLCCCCCCCRCRVVYAGGAGGCGCCAPCPRCSCDCTCACPRCCSCPRCSAACCCAPRCCLCL

SEQ ID NO: 5 - DNA, 5224 bp, Setaria italica
  source 1..5224: mol_type = genomic DNA
ggcggaaacgaaggctcgcttggcgttggcgcagtctcccatctcccctccatcattgccgcccgaaacggcaacccgcccgccaccctctctccctcgctcctccatgtgcccgcctgcagctcgctccatcgccatttaatccgcctcgcctcgctgcttgctggctgcgcgtggcagcagcccggccggcggcagcttgctagtagcgctagctccatcatcattttcttcctcacagctccttgttcttattcactcgtcgcccgctcgccaagcaagccattcacctccagcaccgaacagcttcaccgtcagagtccggccgctcgattatatcggccggcggccgtcagtcagtcgccagcagaaagcaggaacgaaaaaaataatataatggctgctgcgccggcggcacccaggcccaagtcgccgccggcctcgcccgacccgtgcggccgccaccgcctgcagctcgccgtcgacgcgctccaccgggagatcggcttcctcgaggtacccggcttactacctgccatacttgcacacacttggatgttgatcacaactgcgatgcatacacaacgcccgtcttcgtctttgcatcgcaccacctatctgcatgccatgtctttgtcctccccggcccggctcatggtcctggctggcacacccaatgttcttcctgctcctcttgctgcgccgcatttttttttcttttccttttgtttttgttttcagatatggagatgtatagataattgcatactatggataaacaaagcgtctcaggtcaacgaaattactagtaggttgttccagggacggagagctgatgatgaactgggattggagttactaggagctagcgtagcgattgataagaccgttagctttgaaagcttagtttaatccaggcaatctggacagttttaattaggccccgtttgatagggcttctcaaagtgcttctccaccggcttttggtgaagccctaccaaagggtcaatttcaaaacggcttcaccaccaaagtcggggagaagccgcaaaacggcttacactagagaggagaagccgaaaaaagtggcttcaccggcttctcctccctctccaagcattaagtgatcataaaattacatatattgccattgagaagccgttttaccaaatgttttgcaaaacggcttcagcttcactaaaaaagccactccaccgaaaaagccgaagccgaagccgttttatgagaagctgaagctctaccaaacgggaccttaatctctctggctgctggcttttggttttggggcttccgcctctgggcacgtgcggatcgtaccatggatttgcgcgcgcgtcaaggctaactcatctgctcgtcttattccttttgcctaccatttccaccgctccatctcctggacccttcttctagctagggtttaaggggtcctattattatcatcccgcttgcatttccttaatacaatatcatatggcttgcattgttaattaagcttgagcccccgggcggacggtttgactcatcatgtagtaggcgactttgaccaatacactctactagagtaggtactagtatgtagtagggtactttgaccaaaacctcttttttataatagcacctaaaacctttggtaggcatgcatttgttttctcaattactaggaaactggggtgatttgaagctctttattaggtcttattcgattaattcccttttccaagggattatggaaggaatttagagggctgtatacatgatcgtaggcttgtcttctgtaatgacgtgcttaaccagcctgtaactaagctttcttcagcttcacctcatcatgaacttccaagctagcctttgctagctatatattctcgctttaatgttctattgttataatattattatatatacaatgtagcagtcagcagcgcatactggacgacatagagttgcttgtggtggtattgggactgctccagatttccaactactcatccagcactgtccagcctgctcgctgggatgtataaaataatcctgcaaaaatatctgttaattaacactatttactgcccgatgagactgaaaaaacacagccttgcatgccggggccatgtctacgtgccatccatcggtacggcagaaataattaatgtaggtgacaaatttattcctatacaagcaatccatactcttccactaaccgcatagtatgggcttgtttggttgctgcctgccaggtagcaaaaaccaacctcaggcactcaaaatccaacacctggggttgctgcctgagccaggcaggtctccacgacctgaagcaaacaagccctacatgctgtgtattgatccaggagaaaaaaatgtattgatccaggagggaaaaaaacgacagcagtaaatggccttctttgatgcataagatcatccctgccttctgtttgataccaatgctgtttccatcgacgatcttgcattgatgccaatgctggcgtacggccgttagattgcagcatctcgttagaatattggaaagccctcccctgatgatttttctccttcagtactcgactctgaatgtgctgcgtgataaaattatcccagacagcacctgaaaagttgacagcctgagaacatggcttttacttgctgtgcagtattactgttgccaatagcccagtataatattactattattctggaacgctgaaatttaataatttcattgcagggtgaaataagttccattgacggggtccacgctgcctccagatgctgcaaagagtaagctgcagaaatgttactactgcgtacacgggatcttgtattctcacaacaaatattactggaggctaaattaatgaattttcttgttacacttcaaagcatgtattctccattcaagtgtccatgcgtggatgcttaggattagatactagatcaccatatttggtgcaaattagcgattcacaagcagaagctcatatcgcaaactagcttttcactgggaattatacaggaagcaaaattattcagaattgcgccaataatttgtgagctgttttactgtacacaatagtatataacttgtgccaataatagcttttcacttagtagtatataagtatatgtaagatctaattgttcattattatggataatttagtaattattacattgtaaaaagtgatatttcaaatagaatggtctttaaactttaaactttatgctagtgtgaaaaaaataaaattatagtgatgccatttgtgtgttttttattgtgataaccttatactacatatactactcatgtatactatccataccacaggtgaaggttccagtagtatacaattaatcttgatcgtcggatgtttttatactagtccttttcaaacactagtgtagcttagtattgtgtaccataaaagagatttaatttgtatatacaattcctcaaaaatatttttccattaattttattttaatcataaaaatactagtttttaagtttctttttgaataaactgttgttgctaacaatttggagaaaacgaaagaggatctatgttgattgcctctttgatattttttgcagggttgatgagtttgtgggaagaaatcccgatccattcataacgatgtatggattttcaggttgagaatttgtctttagcttggcataaatgttgcattttcttattatctctctttaaaagcagtcagccagagaaacgaagcaatgagcagtctcagcagtttctgaagaagttccggtactttcttagttcccagctcttagtattgttggttcttggggcatataattatgttattttttgttaattaattatccttgagtactgtcagctcagaagaaaaaggacaaattgcatggccaaaaagataatctacatactgtcaaacatggtaccgctcagttgataggcatcattaagacacataaaacacaattaagacacaaaactaacaccagctaaattcacacctatctgtttagggaaagaaaacaacctgtccattttctgaaaaggaagtgcacatatattcagttcaatttgcgtgaagtttctgaccaggacacctgcaaatgtatatatatgattccaagctagcgttttgtagcatcttgtatccatcagctgattcttttagagctagctaataatataagattgcattagaaatctagaacagctttatcagatcatcgctcttagcttttcttatgcaacctgcaacccctccactgtacaaacttcgacaaaacttgtccttcttattgctttaaatatcatctcatcagtgccacactgacagaacgatcaactcattgctaatcacctcaataattgctgaacttgtcgttgcctgcacatctagcaatgatgcttttcagaaacatcaactcattgttaccataattggctcaataattcctgaacttgtcgttaacagtcaagatgatttcaatgaaccagagccaagagctgcctgagctacctgtcgtggatctgctgcggcggcggcgggtgcccgccgttccagctgaagacgacgatgaggccgccgtcggcgagctgctcctgcggcggcgcgcggctgcggaagctctgctcgtccccgtgctgctgctgctgctgctgcaggtgccgcgtggtgtacgccgggtgctgcgcgccgtgcccgcgctgctcgtgcggctgcgcctgcccgcggtgctcgtcgtgcgcctgctgccccacctgcagcgacgcgtgctgcgccccgcgctgctgcctgtgcctatgatggcgttgcgtatgatgcgcgcgtgctgctttaggatcgatgctgctacctgatcgatgaacgaagaagatgatggctttgatgcatggatgttggcagtttgcggtgtcgtctgacaatgcctggtcgtgctggcgaattgagtcaacttattatttcggcccggggtcgcgatttttttcaaaatgacctttatgttcacagaaatcaaacaacagtctccaggatagttatcgggagttcgggacgctagttttttttttactaagctccaaaatatttgaataggaagaagagaaaataccggtttctaattgattttgcagaaagctggcgtcactccttaaaccataaaaacgcagccacttaagaaagtgaggtggagcataaagta

SEQ ID NO: 6 - DNA, 1372 bp, synthetic construct
  source 1..1372: mol_type = other DNA
ggcggaaacgaaggctcgcttggcgttggcgcagtctcccatctcccctccatcattgccgcccgaaacggcaacccgcccgccaccctctctccctcgctcctccatgtgcccgcctgcagctcgctccatcgccatttaatccgcctcgcctcgctgcttgctggctgcgcgtggcagcagcccggccggcggcagcttgctagtagcgctagctccatcatcattttcttcctcacagctccttgttcttattcactcgtcgcccgctcgccaagcaagccattcacctccagcaccgaacagcttcaccgtcagagtccggccgctcgattatatcggccggcggccgtcagtcagtcgccagcagaaagcaggaacgaaaaaaataatataatggctgctgcgccggcggcacccaggcccaagtcgccgccggcctcgcccgacccgtgcggccgccaccgcctgcagctcgccgtcgacgcgctccaccgggagatcggcttcctcgagggtgaaataagttccattgacggggtccacgctgcctccagatgctgcaaagaggttgatgagtttgtgggaagaaatcccgatccattcataacgattcagccagagaaacgaagcaatgagcagtctcagcagtttctgaagaagttccgagccaagagctgcctgagctacctgtcgtggatctgctgcggcggcggcgggtgcccgccgttccagctgaagacgacgatgaggccgccgtcggcgagctgctcctgcggcggcgcgcggctgcggaagctctgctcgtccccgtgctgctgctgctgctgctgcaggtgccgcgtggtgtacgccgggtgctgcgcgccgtgcccgcgctgctcgtgcggctgcgcctgcccgcggtgctcgtcgtgcgcctgctgccccacctgcagcgacgcgtgctgcgccccgcgctgctgcctgtgcctatgatggcgttgcgtatgatgcgcgcgtgctgctttaggatcgatgctgctacctgatcgatgaacgaagaagatgatggctttgatgcatggatgttggcagtttgcggtgtcgtctgacaatgcctggtcgtgctggcgaattgagtcaacttattatttcggcccggggtcgcgatttttttcaaaatgacctttatgttcacagaaatcaaacaacagtctccaggatagttatcgggagttcgggacgctagttttttttttactaagctccaaaatatttgaataggaagaagagaaaataccggtttctaattgattttgcagaaagctggcgtcactccttaaaccataaaaacgcagccacttaagaaagtgaggtggagcataaagta

SEQ ID NO: 7 - DNA, 582 bp, synthetic construct
  misc_feature 1..582: note = Artificial Sequence
  source 1..582: mol_type = other DNA
atggctgctgcgccggcggcacccaggcccaagtcgccgccggcctcgcccgacccgtgcggccgccaccgcctgcagctcgccgtcgacgcgctccaccgggagatcggcttcctcgagggtgaaataagttccattgacggggtccacgctgcctccagatgctgcaaagaggttgatgagtttgtgggaagaaatcccgatccattcataacgattcagccagagaaacgaagcaatgagcagtctcagcagtttctgaagaagttccgagccaagagctgcctgagctacctgtcgtggatctgctgcggcggcggcgggtgcccgccgttccagctgaagacgacgatgaggccgccgtcggcgagctgctcctgcggcggcgcgcggctgcggaagctctgctcgtccccgtgctgctgctgctgctgctgcaggtgccgcgtggtgtacgccgggtgctgcgcgccgtgcccgcgctgctcgtgcggctgcgcctgcccgcggtgctcgtcgtgcgcctgctgccccacctgcagcgacgcgtgctgcgccccgcgctgctgcctgtgcctatga

SEQ ID NO: 8 - protein, 193 aa, Setaria italica
MAAAPAAPRPKSPPASPDPCGRHRLQLAVDALHREIGFLEGEISSIDGVHAASRCCKEVDEFVGRNPDPFITIQPEKRSNEQSQQFLKKFRAKSCLSYLSWICCGGGGCPPFQLKTTMRPPSASCSCGGARLRKLCSSPCCCCCCCRCRVVYAGCCAPCPRCSCGCACPRCSSCACCPTCSDACCAPRCCLCL

SEQ ID NO: 9 - DNA, 411 bp, synthetic construct
  source 1..411: mol_type = other DNA
atggcggcggcgccgaggcccaagtcgccgcctgcctcgccggacccctgcggccgccaccgcctccagctcgccgtcgacgcgctccaccgggagatcggcttcctcgagggcgaaataagttccattgagggggtccacgctgcctccagatgctgcaaagaggttgatgagtttgttggaagtaatccggatccattcctaacgattcagccagagaaaggaagccatgatcaatctcagcagtttctgaagaagttccgagcaaagagctgcctgagctactacctctcgtggatctgctgctgcggcggtggcggtggcggtggcggtggcggtggcggtggtgcccgccgtcgctgcagctcaagaggccagcggcgccgagctgctcctgcgcgccgcggctga

SEQ ID NO: 10 - protein, 136 aa, synthetic construct
MAAAPRPKSPPASPDPCGRHRLQLAVDALHREIGFLEGEISSIEGVHAASRCCKEVDEFVGSNPDPFLTIQPEKGSHDQSQQFLKKFRAKSCLSYYLSWICCCGGGGGGGGGGGGGARRRCSSRGQRRRAAPARRG

SEQ ID NO: 11 - DNA, 204 bp, synthetic construct
  source 1..204: mol_type = other DNA
atggcggcggcgccgaggcccaagtcgccgcctgcctcgccggacccctgcggccgccaccgcctccagctcgccgtcgacgcgctccaccgggagatcagcttcctcgagggcgaaataagttccatgagggggtccacgctgcctccagatgctgcaaagaggttgatgagtttgttggaagtaatccggatccattcctaa

SEQ ID NO: 12 - protein, 67 aa, synthetic construct
MAAAPRPKSPPASPDPCGRHRLQLAVDALHREISFLEGEISSMRGSTLPPDAAKRLMSLLEVIRIHS

SEQ ID NO: 13 - DNA, 126 bp, synthetic construct
  source 1..126: mol_type = other DNA
atggctgctgcgctcggcggcacccaggcccaagtcgccgccggcctcgcccgacccgtgcggccgccaccgcctgcagctcgccgtcgacgcgctccaccgggagatcggcttcctcgagggtga

SEQ ID NO: 14 - protein, 41 aa, synthetic construct
MAAALGGTQAQVAAGLARPVRPPPPAARRRRAPPGDRLPRG

SEQ ID NO: 15 - protein, 67 aa, synthetic construct
LQLAVDALHREISFLEGEISSIEGVHAASRCCKEVDEFVGSNPDPFLTIQPEKGSHDQSQQFLKKFR

SEQ ID NO: 16 - protein, 231 aa, Oryza sativa
MAMAAAPRPKSPPAPPDPCGRHRLQLAVDALHREIGFLEGEINSIEGIHAASRCCREVDEFIGRTPDPFITISSEKRSHDHSHHFLKKFRCLCRASACCLSYLSWICCCSSAAGGCSSSSSSFNLKRPSCCCNCNCNCCSSSSSSCGAALTKSPCRCRRRSCCCRRCCCGGVGVRACASCSCSPPCACCAPPCAGCSCRCTCPCPCPGGCSCACPACRCCCGVPRCCPPCL

SEQ ID NO: 17 - protein, 149 aa, Oryza sativa
MAMAAAPRPKSPPAPPDPCGRHRLQLAVDALHREIGFLEGEINSIEGIHAASRCCREVDEFIGRTPDPFITISSEKRSHDHSHHFLKKFRCLCRASACCLSYLSWICCCSSAAGGCSSSPPPSTSRGRAAAATATATAAPPPPPHVGRR

SEQ ID NO: 18 - protein, 198 aa, Zea mays
MAAAAAPRPKSPPASPDPCGRHRLQLAVDALHREIGFLEGEISSIEGVHAASRCCKEVDEFVGRNPDPFLTIQQERGSHDQSQQFLKKFRGKSCLSYYLSWICGGGWWCPPPLQLKRPPAPSCSCAPRLGKLCSSTASSCCSCCCCRFRVVYAAAGCGCCAPCPRCSCDCTCACPRCCSCACPMCSAACCAPRCCLCL

SEQ ID NO: 19 - DNA, 8913 bp, Oryza sativa
  source 1..8913: mol_type = genomic DNA
ctataaaaaggttgaatggtgcaaagtaagcatagctaaactggttttttatgttacttgttgtggaccctatctacttggattcaaggtttaaacttggtatgggtgcacgtatttacggctaattatttttaaatggtaggtgatgtactgttgacagcgagacgctctcatgatgactttatcaatctaaacatatatatgttggtgcaactttttcaaaggtacttaattcaaaggataggatatttatgtatgagtttgttcataagaacgaatatgccttgataggttgtgcatgggtatctataagagtgagtgtgtgtataatatgatagggtgtgtgtgtgtttttataagaacgactacgcgcatctctatatttcggtacatcccgggtatgccttctctaaaaatgtcttgtgctaaagttgaagacaacttctaaggaaggtatctctcatcttccttgtgtgtccctactagaaaatatttttacaggcggccaaaatatttttttttgcaggcgtccataacctccgcctacacccaaagtcatgcaaaaatcaataatttttgtaggtggctaaatccacccgtttgcgaaaatattttcagcgtaaagaaaattcgccgccctcagccctacccaccgccgccctccccttcggccggatctagggttatggttttgggaggaagagggaggggaggggagctgcgtcaccatcactgaatctggtaggtgagagcgccgccatgagctcaccggtgctgccgccgacctcctgcctacgtgccgccttctccgctgcaagtcgccgctgctgccctccccacgacacagctggatccgggaggggagggcagggaaggttcgctgtcgccgccgccaccctccctacgacgtagccagatatgggtggagaggtaagcgccgccgccgctgcacaacccacgatgccgccgctgcacaacccgcgatgccgtcggccggatttgggaggtggagcgtcgccgccctccgccccgcgccatgtctgaggaaagagagcgatgggagcgccgccggccaccaccgtccccccttccccctccaaccagatctgggaggaagggagggagggaggggagccatcacgtcgttgggagtagatgccgccaccgccctccccctccctgcggccaccacactggggtgcaccgccaccgctaggtagccgcctctggctggatccacgccgaggagaggaaggagagggagagggagagggaggggaggggagatgtggaagttgacttagaaaattttgacgcccggtggtttttaagatactttacctttttgcaggcggatttattaaagaggtccgtctgcaaaaataatgatattttcacggtcggacctcttaagatatctacatgtagaaatctatatttttttacttaaattactgccagggctttgcctcgtagtataattttcataaaaactcaaaatatttacaaattacaaaagaggaaggaaggagaaaaacttataaagattacaatgtttgtaaccaatcaaaaactatctgctttaaaggatctttcattctacacatatgaagaagaacttcttttctaaacgatattcttcatgaaccaaaggagggaagctcattccgaaaatttttcccatttctctgctcgtaggcggataactatctcatctcacggttgttatttttgtaggtggctatttggcttccagagacctactcgtccgggaaaaagaaatgccagcatctagaaaaatagtttttctagtagagtgttgtgagcgtctgcgatcgtggtaaagaaagcaaaaaaggtgaaggacgagtggcatgatacatatgggaaagctgtagactttgacccttgactactcgttggaagtgtgcgtctgcatgcattattgaacggctctgatccccgcggcgcagcggatcggggtcatgtccggatgggcatatcgacgagaaggatccgtccccgacaatctttcaaggcccgtgcccccgtccctcctctcctctgcgcctttccatcatcatttacgcccaaccccaacacatgtacatttcccttggcttgcttccggagaagaaaagagcggccatccactccactctccactctctcccttccatcattacttgcccaaaaacggcaatcccctcccctccatctccatgtgctcttccacctagctccgccattcaaagcaaagcaccaagcttttgcctctccctctaccatgcctgccccctatacatagctgctgcaccgtctctcttcataaatatactagtaggagtagcagagctcatcacttcgatcatctccattatcggaacttcggagtgacatggcaatggcggcggcgccccggcccaagtcgccgccggcgccgcccgacccatgcggccgccaccgcctccagctcgccgtcgacgcgctccaccgcgagatcggattcctcgaggtacaatctatctctatctgtctatatcactaccattcatactccttcgatcttgcttcaaaacaaaaaaatatatatttcctacttcatattcatatacacacgtacggcttgctatctgtcgaattgtttgcttctgcatgcatgcatcactctcattgtaagtttttcccagcttaaaaccactccttttatcttcgttcttcttccttcttgtttttttttaaaaaaacaacaactcatttaatcttcatatagtgtatcatgcatcatttgcttctttgatcagttccccaaaaactgctcctctcttcccagccaaataattaaacttaagcaaacaagcaagttgaactgatgatccaataaaacaaaacaaaaccgatcgaataggaagtcaatggcatataccagctgctatagctgaagccacgaatgcttagcttagctctagtcgatccctgttgactgttcaacacactgcactaacacaccagttaatgagctgattaattaaaccattaaatgagcttaacgggcggctagcttcttcctctgggcccgtgccgatcgtaccatcggtttgcgcgtccctccacctaaactctctgccttgttattccctttgcctagtactacatgcatttgcatcatcatcccatcaccaatagtactacgtttcaactggattttggtggtgtccaaccatatcatatttggttttgttctctagtttactcctacattagtctctagcggttttgtggagtactaaagaaaacaactaatcagccagggtttaacgtttaatcggttggtggttttgttaattaatttcatctactattttagacttcacaggtcttcgagctataagcatcgattgccatgcatcaatcgatgctggtccacgctagtttctgagttctgactagctctcttaattgtgctttgacctactttaattaattaaccagtggctgcgtcactcattgaccaacattgtcatgttacccggactgattttttttttctttaaaaaaacaccggatatattattagttagtgtatatatatgtctgctcaagaagcgcatgcatatagtttctcgtcaaacaaaaaatgtactgtatgctcaaagcatctgttttggaattgtcatattcgcctttataattaaaataattaaaatggtgatgcccagctttttttttcctccaataatttatttattggcttgatttcctgtgctattaggagtaaaactactccgttttaattagcaccatttttaaagcttctaaaattaacctaagtaaagtacgacagtacttgctgtctagctttaaatgttttgggtgttaaaatatccctcagacatcacctgaaaagttgacaggctaaacacatgcccatctccctcgtttacttaaattaattcgaacaaacaactgtatatatatttcttgcagggtgaaataaattcaatcgaagggatccacgctgcctccagatgctgcagagagtaagccagcctgctgtttctttttgtactacttccatttcttctcgtctttactcttaccatgcattcacaaaatatacttacttaccccagtttttgatcatgaactttgaccgttatctttttaagcaacttcaataaaataggtttaaatgcaaatgttatgtacaccattgattataaaaccttggcaaatgaaagtaaaaaaccagcacatttaatttctgaacgttgggagtattattatttttatatcttttactatcatttaatcatagtatcgtgcaagctttttgagtgtaattaggttgcttaaggtaaaaaaatgtaactaggttacatttagtactaaactgaacatttaattagtaatgtttcgttaagtaactgtaatttcaatgcatgcatgtcctcccgtaagagcaagtttaatagtatagccaactactagctccaatttatttatagacaatctaatagctcattcatacaataattacatactacactattaatatctgatcccacctgtcatacacatactgcattttggagtccgtgctatagctgactacaaatctatagtccgctgctcttctctctctttatttatctccttaaaatatgtttgcagctggcttatagcctgctattgtacctgctctgaaaatagtgcagagactgttcaaaaagtcattgcacaataactattcacatggaactgtgaaaagtatatattggaacttactagctagatccttttgggaacatgggaaaagccaagtcacgtgtggaatccctattccctgtgttcttcagctagaagagtgaaaataatgtactactactatacggagatgaaattacagcaggagcagaaagcgggaaaaaaactttaaatcaattaaacaaacctctctctgcaaaattcaacaccagcaacgaacaactcatcaagttcttgtgttatgtaccggccggtaactaattgttgtttgcataagcgaaacggtatatttgcaaacaaaaaataatttatgaataaaacttttatatacatgttcttaattatctcaaaacaaaggttgaaaaataaacttcgatgaaaaaatctcaaaatcaattccaaatttatggtgaaaattttaaattttgtctgataaacataagtataagcaaaaaaaaaagtaaagcaatgtcactatgttaatggtattggtatatatacctttgagtttctgtctgtactttaggagtacatgctacgaacatgttttcttggcttctatttcgttggaattacttgcgtattgtgggccagacgcctggtgaacttcgtcgattgtgtggactaattaagctcacctgaaataagtggttgaaaacaaggctaaagatgattttaatcatggattttggcttggaaatttttttgtagggttgacgaattcatcggaagaactcctgatccattcataacgatgtatggattttcaggtcgagaatttgtctttaacttcgcacgactgttattttttttcttattaattctctgtttacaagcagttcatcggagaagcgaagtcatgatcattctcaccacttcttgaagaagtttcggtactcacttcattcccggatcttaatgtatatatgcatatctgcactgtgctaattggtgtacacattatgtgatcatcagtccaagttaattattacttacaaaactgaactaataaacactagaaaatatgtaacttgcaaagtacatattgaatcagggattcatatatagaactccacctgcagatttcttccaatatatatatgctgtcaccatgttttcacttgtcacctagtacacctttgactgggagactttccttgatgatcgacgtggtcatattcttcagattgatttaatttcagatagaaaaaaatattgtttacttagtttctctccttcagtaagagagatgtgcaagaccagcgatcaaactatatgaactgttcgtttcatgataaaaaaaacatgatatggaataactaggtgattcaacatataatggctgataatccctcgtttcaggagataccatcagttttctacttttctacttttctccatgttctctttttcatgtttgaggtggatcggagcttgtattagatgtttgctcagctcaattattgctgcagatttccctatatagcctccactgtatatatactccctccgattccataatttaatgatattttgaacaatgacgctgtctccaaaatatatctttcactttgttttcctattataatatatacaataaaaaaaatacatatttacttttcttataaatagtttcaaagacaaatctatatatgttgttatataactcttttaaactaaatatttttaaagttatagtcaaagttacaaaagttggacctcaaacatgtataaaacgtcgagaattgtatatctgatcaaccaataatttgtagtgcattgtcttaaaaaaattgcatttctagctatgtatctagataattacaagaaccaggtgaaatcacattttatttttactggaccacgaactcattgtttaattacttccagccttgcactaaataacaataattgaacctggcatcacctgcacaattaatttggacacaaagtaaacatgaatgcaacatacttcgtttcatattgtttgttggtgtaggatttaaattttgtgtcaaaatacttgccgttcttactacattcttaagcactttgagaactaaccttctcttcctacccttcatcaatacagtcatactaactaattggctcttatgccttgaaaaactaattaggatgtatttaatgagggtaaccatgtaatctgccaccagtgaatgcaattttggttcaaaatttcgggcccccgccctaaaaagtcattatctcgatagaatttttttgaatttagtcaaaatttattcaaatttagccaaattgtgttaaatttcaaataatttcagtctaaaaagtgctgaaaatcccgaaatttaggttctaccgaaatggccggaaattttcagcgaaaatcaaacatgaaaaccttgtctgccacatttgttagtttgtgtaaagatttgctagaacgataagtaatttgaagcggatgtatattatatatcccacaaaaccatcaacttgttaattacatgtatatttgtgcatgatgctttcaccactttgtggttgttaacgattaaatcacacgttttattttctcacagctgtttgtgcagagcaagtgcgtgctgcctcagctacctctcctggatctgctgctgcagcagcgccgccggcggctgctcatcctcctcctcctcctccttcaacctcaagaggccgagctgctgctgcaactgcaactgcaactgctgctcctcctcctcctcctcatgtggggcggcgttaacgaagagtccgtgtcgctgccgccgccgcagctgctgctgccgtcgctgctgctgcggcggcgtcggcgtccgcgcgtgcgcgagctgcagctgctccccgccgtgcgcgtgctgcgcgccgccgtgcgcgggatgctcgtgccgctgcacctgcccgtgcccgtgccccggcggctgctcctgcgcgtgcccggcgtgcaggtgctgctgcggcgtccctcgttgctgccccccctgcttgtgatcgatcgatcgattgagcgaagctgcactgattggttaattaattagttctcgatgatgatcgatcgagctgcgcgcgtacttaattagctagctaggttctggtgttaattagttcctcatcgatgcatatgttgattgccttgctctgcttgcggttatctgtaatttggctttgctgccatgatgagtacgtgattgctgatttattttacatatcctctgctatatatatctagctggtagtagctagttttgatctcacttgcaaaactattcatttcctgattttaacaaggtcaaatgacttggttgcctttggttcatactccttagagcaagtataaaggtttatatggaggagagaggtaacaaaaaaaaatcaagagtattggctctcatgcgagagctagcttatcacaagctacaaatcaaatatattaaatgtataagtaagagatagagagaggaataaaattatagctaaccttataggtaatgtattatatatgttaattttaaaataagctaatagtaaaaagtgagctttattattatccttgctcttagtaatggcctcgattcctacattttttttttgagagagtagtacatattttcgcagcgtaatatggacattggccgacgggcctaactattactgggtctaagtgcctaactgagctaaaagccttaaacccaatactgaaaacgaaaaagttcactccgggtccctcatcttgtcgacgggttttaaaatcatcattgaaccggaatacgcattcccaatctttcaaaaccggatcacactaggtctaaggcagtattgctcccgattttggctgatatggtggttgagtcagcgtgagacacacataggctccacatgtcaacgacccaatcttctttcacctctctcccctctcttcacatctttatcgccatccgatcaacgatggcatggaggtcgtcaacaacatccgggcgcacggggtcgaacaccttgtcgtcgtaggggttgaccaaggggccgtcgtgcacggtcttcagcacacgttgatgttgtgcagcgtaacaccaatggggccaacaccgagcaagcctgctctcgcagagtcgtggcgtcgatggttggtggagtgggagagagaaa

SEQ ID NO: 20 - DNA, 696 bp, Oryza sativa
  source 1..696: mol_type = other DNA
atggcaatggcggcggcgccccggcccaagtcgccgccggcgccgcccgacccatgcggccgccaccgcctccagctcgccgtcgacgcgctccaccgcgagatcggattcctcgagggtgaaataaattcaatcgaagggatccacgctgcctccagatgctgcagagaggttgacgaattcatcggaagaactcctgatccattcataacgatttcatcggagaagcgaagtcatgatcattctcaccacttcttgaagaagtttcgctgtttgtgcagagcaagtgcgtgctgcctcagctacctctcctggatctgctgctgcagcagcgccgccggcggctgctcatcctcctcctcctccttcaacctcaagaggccgagctgctgctgcaactgcaactgcaactgctgctcctcctcctcctcctcatgtggggcggcgttaacgaagagtccgtgtcgctgccgccgccgcagctgctgctgccgtcgctgctgctgcggcggcgtcggcgtccgcgcgtgcgcgagctgcagctgctccccgccgtgcgcgtgctgcgcgccgccgtgcgcgggatgctcgtgccgctgcacctgcccgtgcccgtgccccggcggctgctcctgcgcgtgcccggcgtgcaggtgctgctgcggcgtccctcgttgctgccccccctgcttgtga

SEQ ID NO: 21 - DNA, 450 bp, Oryza sativa
  source 1..450: mol_type = other DNA
atggcaatggcggcggcgccccggcccaagtcgccgccggcgccgcccgacccatgcggccgccaccgcctccagctcgccgtcgacgcgctccaccgcgagatcggattcctcgagggtgaaataaattcaatcgaagggatccacgctgcctccagatgctgcagagaggttgacgaattcatcggaagaactcctgatccattcataacgatttcatcggagaagcgaagtcatgatcattctcaccacttcttgaagaagtttcgctgtttgtgcagagcaagtgcgtgctgcctcagctacctctcctggatctgctgctgcagcagcgccgccggcggctgctcatcctctcctcctccttcaacctcaagaggccgagctgctgctgcaactgcaactgcaactgctgctcctcctcctcctcctcatgtggggcggcgttaa

SEQ ID NO: 22 - DNA, 6957 bp, Zea mays
  source 1..6957: mol_type = genomic DNA
actcctagctcactcgtcagcagtctcgtctcagattcagccagtggtggcggactggcggcctagctagctagctatagctgcgcgttgcgagcttgcattgctgcaccggagtagtagtactagtaccgtactaccatctctttctctctctctcgcagcacagccacagcaacactagggtatactacccggaaagagagaaacgaaggcttggctgccgctgtctctctcttctctctctattctctctctctcccctccatcattgatcattgccgcccgaaacggcaacccgccaccatcccactgcacgctgcagctcgccccgtcctcctcctcctcatcctcctctcccccttccccacccccatgtgctggcctgcagcagctcgctccactaccgccatttaatcctcctcgcgctcgctcgctgctgctgcgcctctagcctagtaggctctctgcctcccatcattcgattcctttgcacttgctgtgctggctttgcttgctcttgctctcccctcgccagcaataagctcggccccattccgcttctgccatcaagcccttgccgcgcgcgcacccgcgcactacgtataaccagctagagtagcttggtagctaggctcggctcgctagcgagctcgtcgatccaatccatcgctcgttcgtccactataacaatcgacgacgtgtacgatggcagctgcggcggcgccgaggcccaagtcgccgcctgcctcgccggacccctgcggccgccaccgcctccagctcgccgtcgacgcgctccaccgggagatcggcttcctcgaggtacactacagtacgcattaattaataaatactaatatataaacctcacctgcacatgctcgcataccaccacctctcatatcactccatcatcgattacgctgcactgctcggccgtcttcccgactgccgggcctttccgcattttaatttgcatgcatcacctatatgacaccatatcttatgagcaaggagccctgctgctcccatgagcaaaatcaatccagaccacaccctccacatccaacggtgctgcgtcaggggggtttttgtaaaatgttcagagttgtaaagaacattttacaaaaccccctgacacagcaccgttggatgtggaggttgtggtctggattaattttgctcatgggagcaccaacgctccttgcgcacaagatacgttctctatatgttccttgctgctgagtcagatgcgcttccctttctctctctgtactgaaaatgcaatcgcatgcgtacgtgcagactgcacaaacaaaggatgacgaattgaggataacgaaattgaaattaccagttgcgctagctctggttggtggttctcgctcgatccggccggccgcgagagaaactgaaacgctagcgctagctagcactagcagcagtggggcgattcatgagaccgttaccttgaaagctagcttagtagttctttaatcctctgggcttgctggtttattagtttaggcctccgggcacgtgcggacccgtacgtacgtaccatggatttgcgcgcgtgtcaaggctcaacactctgcgctttatattcgttttgcctaccatttcccacgactacttactccgtcgtctccgtctctctactctatttagctagcttggattcttctttgtcttctgcctgccgctgatactacttctctctctctctctctcaagaatcaaagcactactactactgctgattcagttctgttctatatatctagctactagctagggtttaagggatctaggatctctacatgtacaagaagatacaaggccggcggcccacgctttgattattggtgtcacactgtcacatatgcatctcctagtcctaaatactcctctcgtatggcatgcatgcatggcttttaatacactatccgtatgatcgattacccgcaccatctcatcttatatcaccgcatttgttgattttacttaagattatgtttggttgtttgtatgtgtgagtcacggcctgcataggtggatgcaaaataagccattttgatgttactaaactgcatccacccatacgctcaaaattatcgtatctagccagtcaggacggtgtcagcctgcgcatggacgtgcatccatccaatcaggctgtataatacatcacctaaccgccttatctaggtactctagtatccacgtgtattaagatgaatcgagatgtgaattagtttaatttatacccccgcccccaatccacccccaatacatatggattggggtgaacactatagtacccaaacaaagctgtaggcgtaagtgcacaccttgcattgctggctacaccggcgcacactacactatagtagcaggcgcgtgtagtatatgctaaaacatattcccttcattttttattttgtcatggcttagttcaaaaataaactagcttacgataaatattcaagaacggagctagtatatatttaaacagcctaaatcaaggcttcctttagctcatcaatgaaaaactccgtgcaacctgccctttgctaactttattgttttaatgtattattattattgttgttgttgttgttgttattattattattattattattattattattattattattattattattattattattattattactactattattattattatctgaaacatagtcagcagggcatacgcgacataaagttgcttctgatctgatggtatagggaacgctccacatttgcaactactcatgctgtccagcagcctgctcactagctcagagctatgtcagctacccaagcaataaaatactcctgaaaacatacgttaattaaacactatttactgcccgatgagcatgcagaaacacagctagccctgcgttgccatcggtgtactgcatatatggcagagttattaattaatctaaaccgacaaattcatttagctctccgcaacgtactgatgatgttctacaacatactcatgcatgaccttactggctactgcaccagcagcttgcagcctatatgcatgtctttcttccttgatgcatacatagtcgtactccgcatggccgcatatataacgtacgagttcctttaggctattactagcagcttatccatcttcatctttttatatttaaacttcactcttccaacactacacagtctaccttgtaaaacagttttttagacgaccatacaagtgaattgttaaagactgtttaattttggcggtctattattaggacattagaatttttttgacgtgtcttcccttcgccttcggacgtactccctccgtttctttttatttgtcgctggatagtgcaattttacactattcagcgacaaataaaaagaaacggagggagtaccaccgtcgacgcctcaccaatcaccatggcatggcatgcaccacgtacgagacggtagaactttggtatgtagagcttcgttcacggtcacgcggtggtggagctccatcctgcagactttaaccatgcatgcccgtgagtccgtgacgaccttcttttccctctctctctctccccctactgctatcatggtcatggccatgggctcatggctaacctgcttcggctgtcttcttcaccgctctccccgtccccacaccgtctgacctgtccgttttttttaaaaaaataattaattaattaaagccgccaaatttgaagaagagaagaaggagaagatgagaagagagaaaaactgagtgccgaaagattcgatcggagtgagatgtagactttcggcagcatatagttataaccttaatgattaatctatcacgagattattgatatgccactatatatatgccggcgtacgtacggccgtctgtatataactatatagttgcactatctcgttacattagaataataatactgcaggaaaggccccaatgcaagctgggtttttttttcttcttcagcgagactccaaacttcgaaggtgccgcgtgtgttttaaaaatatcccagacaatgcctgaaaagttgacagctgaacacatggctttttacttgaccagtatacagtggtatttactatttaccaacatagtattactatatcattaccccctgaaacgctcggtttggtttccgttgctgcatgcagggcgaaataagttccatcgagggggtccacgctgcctccagatgctgcaaagagtaagctgaacacatgctccgttatatattatattgctacgtacgtgtgttatatacatacaggggggcggcccttgttacatttacatgcatgcgatccacgaggatggcttaaaacgctagtatcagaaataatcgatgaactttctctttagtttaattaattgcaaaacatgcatgttctcattcagttgtccatccatccatccatacattgttgttctttggataagatatatactagatagccatggtcggataactagctagttttaggtttctgtctgaatgaatcgctgctacaacaatatatatactgaccatgtgggcaaataaccaaagaagattctctgctgattgcctctctttgataattctctgcagggttgatgagttcgttggaaggaacccggatccgttcctaacgatgtatggattttcaggttgtgaattcgtcctttaacctagcgtgaatggtgccgttttcttaacatatatatctgttcaaaagcagtcagcaagagagaggaagccacgatcaatctcagcagtttctgaagaagttccggtacttccgttgctcacagctcttaataattattatagtaatcgccggttcttgcccattagtaaactacacgtctttattaatgttattttgttgtcaattgtgtattaccctagcactagctagcagatcgatcacatgaaaaaagactaatttgtacgagagaaaaaaaaatcctactatcatcacccaaatcatttttaggtaggatgggagaagcaacataaacaactagcctctgcaaatcgtcatatataagacagaaaactatcacccagagctaataaaccccgaaatgaatgagacatagatatctcaattattactgtctactgagaccacaacctgtccattttctgaaaaagtatgaaaagggggcatattcattcagtgcaagctgtatgaaagcttggtgatcatccgcgaacgagctgagcctcagtcccactccctcccaccagcgcgcgtacgtacgtgtacgtcctagcagcttcaacggcttgtatcagatctctgctgctttttttttacccctttgcagcctgcaacccgtactgcactactccctccgttcctttttatttgtcgctggatagtgtaattttgcactatccagcgacaaataaaaagaaacggagggagtacaaactttcagagtaatcagcatgtcgtctactttaaactgccatgccgtcaacatcacacgttgtcagcccagccctgcagagcaatcagtttgtcgttgtctgcgctaacacttaatcgagcatctatagtgctgcttttcagaaccgtcacctcattgttactataattgtttcaactatttcctcatcaccacgtcgtcagaaaaaccatcgtcgtctgctgctggctagctgaataggagtgcatcagtaaaacctgaaaataatgctgctgctactgtttgttgctaatgctagctagctactgcatagcttgcattgagttgggtgaattaatgattggcaatcagatgaatgaagttgagttttgtgcatgcatgcatgcagaggaaagagctgcctgagctactacctctcgtggatctgcggtggcgggtggtggtgcccgccgccgttgcagctcaagaggccaccggcgccgagctgctcctgcgcgccgcggctggggaagctctgctcctccaccgcctcctcctgctgcagctgttgctgctgccggttccgcgtcgtgtacgccgccgccggctgcgggtgctgcgcgccgtgcccgcgctgctcgtgcgactgcacctgcgcctgcccgcgctgctgctcctgcgcctgccccatgtgcagcgccgcgtgctgcgccccgcgctgctgcctgtgcctatgatggccatgagaaattttgcgtgcatgctagtagtagtagtacccggagatgatgatggatgactggcattttgcagcatggttttttttttggtttagtttcggcgacacgagtactgttgtaactggctgtacgtggctgatctggttagtgctggtgaatggtgtcgattccacattttccgctgagagttgccatgacatgcagccctactagtcttacgtacagcacaaaagatgcttagattccactcagtccttcggttattgcaatttaatgtggacgggagtgtattgaaatgtattgggatagattttgacttgctattaatttaaaccgacccaatatcactcaatccatatggattaacagtgcgaacaaaccctaatggcctgttcgtttatgccggatttcactaaaaatcgttccagctgatcaaaccttatgcggctgattggttggactgctgcccgtggcctgcctcaggcgagcatgccccgtgggaaacagaagtttctagcgtacccgcgcatgcagctttaagagatatttttttgctagcgttgcatctgccagtacaacttgatctcgttttatgcacttaaccaaacaaaggctagtgttgggtcaacgcacgcgctcaaacgggacggacatgtgcagcataccaatcagaaggtatataaattagacaaacaatccagctagaaattatttcgggtgctcaatccactggaaccgaacatgccccaagatggtttaagactagaaaaagac

SEQ ID NO: 23 - DNA, 597 bp, Zea mays
  source 1..597: mol_type = other DNA
atggcagctgcggcggcgccgaggcccaagtcgccgcctgcctcgccggacccctgcggccgccaccgcctccagctcgccgtcgacgcgctccaccgggagatcggcttcctcgagggcgaaataagttccatcgagggggtccacgctgcctccagatgctgcaaagaggttgatgagttcgttggaaggaacccggatccgttcctaacgattcagcaagagagaggaagccacgatcaatctcagcagtttctgaagaagttccgaggaaagagctgcctgagctactacctctcgtggatctgcggtggcgggtggtggtgcccgccgccgttgcagctcaagaggccaccggcgccgagctgctcctgcgcgccgcggctggggaagctctgctcctccaccgcctcctcctgctgcagctgttgctgctgccggttccgcgtcgtgtacgccgccgccggctgcgggtgctgcgcgccgtgcccgcgctgctcgtgcgactgcacctgcgcctgcccgcgctgctgctcctgcgcctgccccatgtgcagcgccgcgtgctgcgccccgcgctgctgcctgtgcctatga

SEQ ID NO: 24 - DNA, 20 bp, synthetic construct
  source 1..20: mol_type = other DNA
gccccggcccaagtcgccgc

SEQ ID NO: 25 - DNA, 20 bp, synthetic construct
  source 1..20: mol_type = other DNA
gacgcgctccaccgcgagat

SEQ ID NO: 26 - DNA, 20 bp, synthetic construct
  source 1..20: mol_type = other DNA
ccgggagatcggcttcctcg

SEQ ID NO: 27 - RNA, 103 nt, synthetic construct
  source 1..103: mol_type = other RNA
gccccggcccaagtcgccgcgttttagagctagaaatagcaagttaaaataaggctagtccgttatcaacttgaaaaagtggcaccgagtcggtgcttttttt

SEQ ID NO: 28 - RNA, 103 nt, synthetic construct
  source 1..103: mol_type = other RNA
gacgcgctccaccgcgagatgttttagagctagaaatagcaagttaaaataaggctagtccgttatcaacttgaaaaagtggcaccgagtcggtgcttttttt

SEQ ID NO: 29 - RNA, 103 nt, synthetic construct
  source 1..103: mol_type = other RNA
cgaggaagccgatctcccgggttttagagctagaaatagcaagttaaaataaggctagtccgttatcaacttgaaaaagtggcaccgagtcggtgcttttttt

SEQ ID NO: 30 - DNA, 698 bp, Oryza sativa
  source 1..698: mol_type = other DNA
atggcaatggcggcggcgccccggcccaagtcgcacgccggcgccgcccgacccatgcggccgccaccgcctccagctcgccgtcgacgcgctccaccgcgaagatcggattcctcgagggtgaaataaattcaatcgaagggatccacgctgcctccagatgctgcagagaggttgacgaattcatcggaagaactcctgatccattcataacgatttcatcggagaagcgaagtcatgatcattctcaccacttcttgaagaagtttcgctgtttgtgcagagcaagtgcgtgctgcctcagctacctctcctggatctgctgctgcagcagcgccgccggcggctgctcatcctcctcctcctccttcaacctcaagaggccgagctgctgctgcaactgcaactgcaactgctgctcctcctcctcctcctcatgtggggcggcgttaacgaagagtccgtgtcgctgccgccgccgcagctgctgctgccgtcgctgctgctgcggcggcgtcggcgtccgcgcgtgcgcgagctgcagctgctccccgccgtgcgcgtgctgcgcgccgccgtgcgcgggatgctcgtgccgctgcacctgcccgtgcccgtgccccggcggctgctcctgcgcgtgcccggcgtgcaggtgctgctgcggcgtccctcgttgctgccccccctgcttgtga

SEQ ID NO: 31 - DNA, 563 bp, Zea mays
  source 1..563: mol_type = other DNA
atggcagctgcggcggcgccgaggcccaagtcgccgcctgcctcgccggacccctgcggccgccaccgctgggcttcctcgagggcgaaataagttccatcgagggggtccacgctgcctccagatgctgcaaagaggttgatgagttcgttggaaggaacccggatccgttcctaacgattcagcaagagagaggaagccacgatcaatctcagcagtttctgaagaagttccgaggaaagagctgcctgagctactacctctcgtggatctgcggtggcgggtggtggtgcccgccgccgttgcagctcaagaggccaccggcgccgagctgctcctgcgcgccgcggctggggaagctctgctcctccaccgcctcctcctgctgcagctgttgctgctgccggttccgcgtcgtgtacgccgccgccggctgcgggtgctgcgcgccgtgcccgcgctgctcgtgcgactgcacctgcgcctgcccgcgctgctgctcctgcgcctgccccatgtgcagcgccgcgtgctgcgccccgcgctgctgcctgtgcctatga

SEQ ID NO: 32 - protein, 42 aa, Oryza sativa
MAMAAAPRPKSHAGAARPMRPPPPPARRRRAPPRRSDSSRVK

SEQ ID NO: 33 - protein, 30 aa, Zea mays
MAAAAAPRPKSPPASPDPCGRHRWASSRAK

SEQ ID NO: 34 - DNA, 811 bp, synthetic construct
  source 1..811: mol_type = other DNA
ggatccacgctgcctccagatgctgcagagaggttgacgaattcatcggaagaactcctgatccattcataacgatttcatcggagaagcgaagtcatgatcattctcaccacttcttgaagaagtttcgctgtttgtgcagagcaagtgcgtgctgcctcagctacctctcctggatctgctgctgcagcagcgccgccggcggctgctcatcctcctcctcctccttcaacctcaagaggccgagctgctgctgcaactgcaactgcaactgctgctcctcctcctcctcctcatgtggggcggcgttaacgaagagtccgtgtcgctgccgccgccgcagctgctgctgccgtcgctgctgctgcggcggcgtcggcgtccgcgcgtgcgcgagctgcagctgctccccgccgtgcgcgtgctgcgcgccgccgtgcgcgggatgctcgtgccgctgcacctgcccgtgcccgtgccccggcggctgctcctgcgcgtgcccggcgtgcaggtgctgctgcggcgtccctcgttgctgccccccctgcttgtgatcgatcgatcgattgagcgaagctgcactgattggttaattaattagttctcgatgatgatcgatcgagctgcgcgcgtacttaattagctagctaggttctggtgttaattagttcctcatcgatgcatatgttgattgccttgctctgcttgcggttatctgtaatttggctttgctgccatgatgagtacgtgattgctgatttattttacaaaaaaaaaaaaaaaaaaactcgagggggggcccggtacc

SEQ ID NO: 35 - DNA, 823 bp, synthetic construct
  source 1..823: mol_type = other DNA
gagctcggatccacgctgcctccagatgctgcagagaggttgacgaattcatcggaagaactcctgatccattcataacgatttcatcggagaagcgaagtcatgatcattctcaccacttcttgaagaagtttcgctgtttgtgcagagcaagtgcgtgctgcctcagctacctctcctggatctgctgctgcagcagcgccgccggcggctgctcatcctcctcctcctccttcaacctcaagaggccgagctgctgctgcaactgcaactgcaactgctgctcctcctcctcctcctcatgtggggcggcgttaacgaagagtccgtgtcgctgccgccgccgcagctgctgctgccgtcgctgctgctgcggcggcgtcggcgtccgcgcgtgcgcgagctgcagctgctccccgccgtgcgcgtgctgcgcgccgccgtgcgcgggatgctcgtgccgctgcacctgcccgtgcccgtgccccggcggctgctcctgcgcgtgcccggcgtgcaggtgctgctgcggcgtccctcgttgctgccccccctgcttgtgatcgatcgatcgattgagcgaagctgcactgattggttaattaattagttctcgatgatgatcgatcgagctgcgcgcgtacttaattagctagctaggttctggtgttaattagttcctcatcgatgcatatgttgattgccttgctctgcttgcggttatctgtaatttggctttgctgccatgatgagtacgtgattgctgatttattttacaaaaaaaaaaaaaaaaaaactcgagggggggcccggtaccactagt

SEQ ID NO: 36 - DNA, 30 bp, synthetic construct
  source 1..30: mol_type = other DNA
ggtaccaccatggcaatggcggcggcgccc

SEQ ID NO: 37 - DNA, 27 bp, synthetic construct
  source 1..27: mol_type = other DNA
agatctcaagcagggggggcagcaacg

SEQ ID NO: 38 - DNA, 25 bp, synthetic construct
  source 1..25: mol_type = other DNA
agatctacgccgccccacatgagga

SEQ ID NO: 39 - DNA, 17 bp, synthetic construct
  source 1..17: mol_type = other DNA
ccgcgagatcggattcc

SEQ ID NO: 40 - DNA, 19 bp, synthetic construct
  source 1..19: mol_type = other DNA
cgtggatcccttcgattga

SEQ ID NO: 41 - DNA, 19 bp, synthetic construct
  source 1..19: mol_type = other DNA
aaccagctgaggcccaaga

SEQ ID NO: 42 - DNA, 24 bp, synthetic construct
  source 1..24: mol_type = other DNA
acgattgatttaaccagtccatga

SEQ ID NO: 43 - DNA, 38 bp, synthetic construct
  source 1..38: mol_type = other DNA
ttcagaggtctctctcgcactggaatcggcagcaaagg

SEQ ID NO: 44 - DNA, 37 bp, synthetic construct
  source 1..37: mol_type = other DNA
agcgtgggtctcgtcagggtccatccactccaagctc

SEQ ID NO: 45 - DNA, 38 bp, synthetic construct
  source 1..38: mol_type = other DNA
ttcagaggtctctctgacactggaatcggcagcaaagg

SEQ ID NO: 46 - DNA, 37 bp, synthetic construct
  source 1..37: mol_type = other DNA
agcgtgggtctcgaccgggtccatccactccaagctc

SEQ ID NO: 47 - DNA, 41 bp, synthetic construct
  source 1..41: mol_type = other DNA
aacggattcagccggtctcggttttagagctagaaatagca

SEQ ID NO: 48 - DNA, 40 bp, synthetic construct
  source 1..40: mol_type = other DNA
cgagaccggctgaatccgtttgccacggatcatctgcaca

SEQ ID NO: 49 - DNA, 41 bp, synthetic construct
  source 1..41: mol_type = other DNA
gggacttgaacggattcagcgttttagagctagaaatagca

SEQ ID NO: 50 - DNA, 41 bp, synthetic construct
  source 1..41: mol_type = other DNA
gctgaatccgttcaagtccccggcagccaagccagcacccg

SEQ ID NO: 51 - DNA, 20 bp, synthetic construct
  source 1..20: mol_type = other DNA
tacatagctgctgcaccgtc

SEQ ID NO: 52 - DNA, 22 bp, synthetic construct
  source 1..22: mol_type = other DNA
gaagcaagatcgaaggagtatg

SEQ ID NO: 53 - DNA, 21 bp, synthetic construct
  source 1..21: mol_type = other DNA
actataacaatcgacgacgtg

SEQ ID NO: 54 - DNA, 20 bp, synthetic construct
  source 1..20: mol_type = other DNA
agcagtgcagcgtaatcgat

SEQ ID NO: 55 - protein, 121 aa, Triticum aestivum
MAAPRPKSPLDPCGRRRLQLAVDALHRQISFLEGEISSIEGLHAASICCKEVDEFIGKNADPFITISSEKGNADQSHRSPKKIRTRWACLSCSSSCACPACAGCGPACCGGVPRPRCCLCS

SEQ ID NO: 56 - protein, 170 aa, Triticum aestivum
MAAPRPKSPLDPCGRRRLQLAVDALHRQISFLEGEISSIEGLHAASICCKEVDEFIGKNADPFITISSEKGNADQSHRSPKKIRTRWACLSCFPWICGGGCSAVQLKGPSCCCGCPRCCAGSGGCGGGGPSCGCSCSCAGCSSSCACPACAGCGPVCCGGVPRPRCCLCS

SEQ ID NO: 57 - protein, 169 aa, Triticum aestivum
MAAPRPKSPLDPCGRHRLQLAVDALHRQISFLEGEINSIEGLHAASICCKEVDEFIGKNADPFITISSEKGNAEQSHPFPKKIRTRWACLSCFPWICGGGCSAVQLKGPSCCCGCPRCCAGSGGCGGGPSCGCSCSCAGCSSSCACPACAGCGTVCCGGVPRPRCCLCS

SEQ ID NO: 58 - protein, 169 aa, Triticum aestivum
MAAPRPKSPLDPCGRHRLQLAVDALHRQISFLEGEISSIEGLHAASICCKEVDEFIGKNADPFITISSEKGNADQSHRFPKKIRTRWACLSCFPWICGGGCSAVQLKGPSCCCGCPRCCVGSGGCGGGPSCGCSCSCAGCSSSCACPACTGCGATCCGGVPRPRCCLCS

SEQ ID NO: 59 - DNA, 6109 bp, Triticum aestivum
  source 1..6109: mol_type = genomic DNA
acccgtacccacataataaagcctcaaaaaagggtaataagcactttccacccctcatctgactaacctgaatatctttcaatgaaaaaacggtctaaacataacatcttggagctcctgcagcccctgaagtcgacttgccacagcagcctcctcatgtcgtggagcaagggcgtgttatacctatggatgcacctactgtcgctacaaactcaagtgcaaacgaggacacatttccttcctgggacgatgttcttacccaaccttatagcccaagacatacacaaggtttgcaattgtgttaactaccgcatattctgtctatggtcaatatttttacaaggtaccactaacttctttgctcttatcgaaccgacagacttaggcgctgcaaataatagctctagtagcatagagtgccaatctggaacttggccagcttctgatgcgattgggcaagaaatttggccaacttccgatgcgattgagcaagagattgtacttccagcattgcatggtgagcttctctttgtacatcatacatctatacagtttgttaacatgctggagactgttgtggacatctatacaaatttttaataaattcacaaccctatggattatggtggcaactgttgtggttatattaagattcatctggttatattagatgtaaactggaatgcatgatacgtgcaaattctgttccacttgttaaagagttgatcaatcggtcacattcatttaattctatttccacttgttaaagtgcatgatatgatatgtgcaaattctattccgcatatctggcagatgtgcatgatatgagagtacactaacatggctagtttaactccattcattatttttgaacctttcagtctggtgtcaatcagtgtgtttctccctgtttaactctgttggagccttctgcatttggcttaccttcattctgtccatcaaattttgcaaatttgccgtaatgctcttgatgtctggatgataaatgatgtaccctgttacaatatgaaaaatgtaatgatgtgcagtatttcgtattaccctttgatggccttgatatgaaacaatgtgttcatgtttttggtttatcagttccctatctgacatttgttttgtacttattctggtgaacttgcagccatgccagactttgctcaagtatacaactttcttgggggcgtatttgatccagatacaacaggacatttgcagaagttgagagagatggatcctatagatgctgaaacggtattgccatttgctaatctcccctctgtaattgattcatctgccctgctataaagctattgatttctgcaagaatgaaggccaggtcgttttgtttgcattgtttagtgacatcccagttactcgcaagctgctatgtcagtacctcgattcagtcagtgtcatataaaaacctatgaaactattccggacaggtgtattctggggtgaagtttaattactcattgttgcacattgctccttatcaaggatatttagttgctgtaattcttatatacgttacttatatgttcgcagcaaatgcagatgctcactttgttcattcttttgcaagtcgaaatgtactgtcagggattcacttttattacctaataacttctactcacttacaatgatctttttatgggctaatctccaggtactacaactgatgaaaaatctatcggtaaacctgtctagcccggactttgagacacacgtaaggacttaaactcgtttttctttcatgccacctgcttttaatatttatttactctgtgatccacaatatttgtctacctattgtgaacaaagtagctgacactgctgtagaacagaagtagcttgcgctattgtcgaacagttctcaaactgttttccatgccgacagccacattagtgtcacaactattctgttcaactaattactttagcataataattttgcttgatttgtgaatctgccgcaacttgttgtgtactaatccgttagctttgaagattctttttgtgactgttctgaactgaatcaacacaaaaccagttacgttccactccatgctgcacattaatcgaatctttgctatcctgttatgcaaaataaaagtaaatggcaggatggtgttttgcttgctgggtgctttgtcagtaatgaagcatattgtataatattgtgcttcatgagatcatttcatgctccctaatctgttgcaagtcatctttcttggaaatccagctgagcatacagcacccctgactgttgcagaggaggatgctctcgtcgcacggtggcggcatgggccaagccaggcacgagagcgtaggagatccggggtctctactcagacccttcgcgttccattcatgtaatctctagtcaccctcttcgtgatccacgctcacgacatcgctaatttcactaagcgcatggaccgctctgcagggtaaggagcagatgaggaaagcgtacggtacatcgttacattgttgtcaaagttgcgacatgttaatgcatcgccgcctatttttgtacatatttcttggagatgttctcgggctcttaggcctgggaacttttacctatccggcttgtggcatgtatagtcaggacatggttgcgcatgttgttttggagaggggagaggggaaagtgtttattacccatgaaggaagaccgttcttgtgtgtaattctttgtttagagatgattttttttgagggattgtttcgagatgattttgtttgtaagtctattatctacatcgtcatgaggacggatttggactatatatttgtagtgaacgcttttcgtctcgttgtctactttgtagttcgtgttcatcaataatgttttctcaatcgaaaaatgataatcaagtcatcaatcagctagtaacattttgcgaaaattgctttgtttttttaatcccaaaaaaatggacttgattggctacttcagagaaggaaaaaatgctaatgattttttttttagaatggagtcaaaggaaggagagaagtggtggtcgtggtaggcccgcgagggtccatccggcccagcagccttggctaaccctacccactacccaccacctacctaccctacctaccccagtctccagccctacagaccgacgacttcctgtctcctcctcccggcggccacctccgtccatcgctctccccccctctctccgccgccgccgccgggcctctgaatccccttgctcctccttccgaggaggcagcacatcagcatcagcggcgtgatctgatccggtctgctagactgatcgctcattcactccattgatgacgctctctgcttccccgcactccccgccgccgtccacctcccgccccccgctcctctcctggcccagctagccagccggcgcgagcgagccctccatccaccaccgtccagctagccaggctcctccgaggccggcaatggcggcgcccaggcccaagtccccgctcgacccctgcggccgccgccggctgcagctcgccgtcgacgcgctccaccgccagatcagcttcctcgaggtacccaagctcctgaccttaatcgctcttcctggttggctcccgcaccacagccctgttacatggtgccctgctgctgctgttactctgttattattacccaagaaaattactaatttgcaattgagcctacacaacaaatgattaccatttttatacagtagtaggtattagagtctgttaccagcacacagtttagttgtcacttctaagtcagttccaggtgtccctttctactactactactactactggtagtagattctaagtgttagaatatcccacacagcagcctgaaaagttggcacatgccctctcctccttgtactctagcttgtagctactgcttttttttcagtaaaaaataatttctctggaattcaacaagctgttctctctgtgccagggggagatcagttccattgaagggctccatgctgcctccatatgctgcaaagagtaagctgctgtcttgttcaattcttatactatcaatttcctttttgaagtttaagaggtaacttggggagtgaaaacaagaaacagttctccggctccaagaatggggaacaatccattgcaagtgagcccatgtttgaacaaagtcgtacagacaggtgcaacacatctacatggatgtgtgaaagcaacacagattttagttaaaacctacaaatggaagtttagcaaatccggtaatgattgaaaatagtatacacgtacagttgaacattatgcaaatctggtaacgatagtttaggaggtttggttacttgaaatttgattaggatgattgattgtatttaaacatatgaatatgttttagaattggacctaaattatatagggttgcggtgactgtacaggcagttcttgtatttctgtctccagtcttagtgccactgtttactttattcttcttcataaatgaatatcagcgtaagctgcagacgtagcagtcaggtcatgtgtgcaaaagccaaaaatatgtaattcttcttcatatgtcaatcagttcacatagttgtgccaattttaagttaattttcttcttgttcatgacatatggagctgattgatcgctgtttgcccctttgataaaatttgcagggtcgatgagttcataggaaagaatgccgatccattcataacgatgtatggattttcaggttgagaaattgtcttggcctacaagatatctttgttcttactagtatctctctttacaagcttgcagttcatctgagaaggggaacgccgatcaatctcatcgctccccaaagaagattcggtaatcattttcttcccgcagatcctaatatatgtcgacggttctctcgaaagaaaatgatgccggatcaacccaagctgatgcaaattgttgtactaaactgcacatgtttatatttaaaagttttggaatattgataattttcccagttttacatctgcgctctacaaaccataacaattatgaatcagccagcaatgcatgcatcgatttgaactcggttttagtagtctcgttgtaatgaactgactgaggggaatgttaatgtttgtgcctgcagaacccggtgggcgtgtttgagctgcttcccgtggatctgcggcggcgggtgctctgccgtccagctcaaggggccgagctgctgctgcggatgcccccgctgctgcgcggggagcgggggctgcggcggcggcgggccctcgtgtggctgctcgtgctcctgcgccggctgctcctcctcttgcgcgtgccctgcctgtgccggctgcggccccgcgtgctgcggcggtgtccctcgccctcgctgctgcctgtgttcatgaggcgccgtattgagatttttcttcttcttcttcgttcttgctgtgtgcgttgtgcgttgcctgtaggctcaggctcggctggtgtttttgacgggcatgacgatgatgatgtatttctggctcagttggcgctgtggatttgcctggttttacttgccgcagagaaggatcgcttgggccttcgtccttcctatagtctcctacgttgtaattaaagctttggagataactgcagaatgaacgggttttctcactgatgagtgatgatggtcagggtgtatatatgcccagttagagcatggtttccggtttttgtcccaaaaaatgacccgagcagatcctgcattttcgccaaaccgtaaatattttgcctttagccataaaatgactcggcgccacgcgacgacacgtcgctggctagccaggagccgcttggtgccgttgccggggcggcggccaggacccaattgtaatctacatttgcttttagggggctttatgctatttttaggaccctgttgtaaatttaactactaacaagtgggcttctacatatgacccgaaacgagttttatgagatctgttcaggcggaacacacaccttagtgctatagtatatccgaaacgattttacagtaggagattttacgagacctgccagagatgctcttagcggatctccaacgtcgagccacaaaac

SEQ ID NO: 60 - DNA, 5475 bp, Triticum aestivum
  source 1..5475: mol_type = genomic DNA
caattggtgttaactaccgcatattctgtctatggtcaatatttttacaaggtaccactaacttcttcgttcttattaaactgacagatttaggcgctgcgaataatagctctagtagcatagagtgccaatctggaacttgggcaacttctgatgcgattgagcaagaaacttggccaacttccgatgcgattgagcaagagattgtacttccagcactgcatggtgagcttctctttgtacatcatacatctatacagtttgttaacatgctggagactcttgtggacatctatacatttttttaataaattcacaaccgtatggaatatggtggcaactgttgtggctatagtaaggttcatctggttatattagatgtaaactggaatgcatgatacgtgcaaattctgttccacttgttgaagtgctgatcaatcggtcacattcatttaattctattccacttgttaaagtgcatgatatgatatgtgcaaattctattccacatatctggcagatatgcatgatatgagagtacactaacaaggctagtttaactaccattcattatattttgaaccttttagtctgtggtgtcaatctgtgtgttttcccctgtttaactctgttggagctttctgcatttggcttaccttcattctgtccttcaaattttgtgaatttgccgtaatgctcttgatgcctggatgataaatgatgcaccctgttacaatgtgaaaatgtaatgatgtgcagtatttcatattaccctttgatggccttgatattaattaatgtgtttaccattttggtttatcagttcccagtctgacattcgttttctacttattctggttaacttgcagccatgccagactttgctcaagtatacaactttcttgggggcgtatttgatccagatacaacaggacatttgcagaagttgagagagatggatcctatagatgctgaaacggtatgccatttgctaatctcccctctataattgattcatctgccctgctataaagctattaatttctgcaagcatgaaggccaggtcattttgtttgcattgtttagtaactgacatcccagttactcgcaagctgctatgtcagtaccttgattcagtcagtgtgatataaaaacctatgaaactattccggacaggtggattctggggtgaagtttaattactcattgttgcacattgctccttatcaaggatatttagttgttgtaattcttatatacgttacttatgtgttcgcagcaaatgcagatgcttactttgttcattcttttgcaagtcgaaatgtactgtcagggattcacttttattacctaataacttctactcacttacaatgatctttttatgggctaatctccaggtactacaactgatgaaaaatctatcggtaaacctgtctggcccggactttgagacacatgtaaggacttaaattattttttcttgcacaccatctgcttaatatttactctgtgatccaacatatttgtctacgtactgtaaacaaagtagctgatactactgtagaacagaagtagcctgcgctatcgtcaaacagttctcgaattgttttccatgccgacatccccattaatgtcacaactattctgttcaactaattattttagcatgacaattttgtttaattaaatctgccgcaacttgtagcactaatccattagctccgaagattctttttgtgactgttctgaactgaaattaacacggaacagttatggtccactccatgctgcacaataattgaattattgctatcttgttatgtactccatccatcccataatataagagttttttttacactagtgtagtgtcaaaaacgctcttatattatgggacagagggagtaaaatagaagtaaatggcaggatggtgtttttcttgctaggtgctttgtcaggaatgaagcatatagtatataatattgtgtttcatgagatcatttcatgttccctaatctgttgcaatccatctttcttggaaatccagctgagcatacagcatccctgactgctgcagaggaggctgctctcgtcgcacggtgccggcaagggccaagccaggcacgagagtgtaggagatccggggtctactcagattgttcacgttccattcatgtaatctctagtcaccctcttcgtgagcacggatccgtgctcacgacatcgctaatttcgataagcgcatgggccgctctgcagggtaaaggagcaaatgaggggagaggacagtacattgttgttagaggtgcgacatgttgcagcatcgcttgttgtcagaggtgcaacatgttgctgcatcaccgcgtgcctatgtttgtaaatagttcttcgagatgttttcgggctcttagatgtgggaacttatatctatctgtcgatgaggctttgtggcatgtatagtcaaggcatggttgcgcatgttgtttcggagagggaggaggggaaagtgttattacccacgaagaaataccgttcttgtgtgtaattctttgtttggagatgattttgtaagtctattatcgtcatcgtcatgaggggatttgggctatgtatgcagtgaactcttttcgcctcgtcgtctactagctagttcgtgttgttaaatagttgattatgttttttcgaacaaataatgatactcaaatcatcaatcagctagtaacattttgcaaaattgccttgatttttaatcccaaaaaatggacttgactagctacttcagaaaagaacaaaatgctattagcaatatttttgttttagaatgaagtcattttttttctttttgagacaagaatggagtcaatggaaggagagaagaagtgctggtcctggtaggcccgccacgatccagccggcccagcagccttgcttaaccctacccgccacctaccacctaccccagtggccagccctatagacgacttcctgtctccttccggcggccacctccgtccgtctctctccccctctccgccgccgccgccgccgcccggcctctaaacccccttggtcctccccccagcccccacaccggcggcgtgatctgctctggtctgctagattgatcgcccattcattccattgatgacgctctctggctccccgcactccccgccgccgtccaccagccgccccccgctcctctcctggcctagctagccagccagccggcgcgagcgacccctccatccaccaccgtccagctagctagctagctccttcgaggccggcaatggcggcgcccaggcccaagtccccgctcgacccctgcggccgccaccgcctgcagctcgccgtcgacgcgctccaccgccagatcagcttcctcgaggtaactaagctcctaatcccgactgactcgcacaatataccctgtaaagatgatgctctgctgttactctgttatcacgcaagaaaaactactgactagcagttgagcatacacaaatgatcaccatttctttacaggtgtctaggctctagagtctgttaccagcacacagtttagttgtcacttccaacttctaagtcaattccaagtgttcctttctgctactactgctactagattcttagtgttaaaatatcccagacagcaacctgaaaagttgacacatgcccttttctccctgtactatattgctagttgctagctagtagctactgctttttttcagtaaaaaataatttctctggaattcaagaaactattcttatgtgccagggggagatcaattccattgaagggctccatgctgcctccatatgctgcaaagagtaagctgctgtgtttgttcagttcttatatcagttttgtttttgaaatttaagagataaggtagggaacgaaaacaagaaatagttctccggctgcaagaatggagaacaatccattacaagtgagcccatgtttgaataaagtcgtacagatgcgtgcaacacatctacatggatgtgtgaaggcaacacaggttttagttaaaacctctaaatggaggtttcacattttagccaagaaatgtcgctctcagtggaaacttgtacaatacattgctatctactaccatgggcttccacaccaatgattgaaaatagtatacacttacaactgaacattatgcaaatctggtagcaatagtttgggaggtttgattacttggaatttgattaggatgattgattgtatttagatatatgaatatgttttggaactggacttcaattatatatggttgcgttgtacaggcagtgcttttattttcatctccagtctcagtgccactgattactttattcttcttcataaatgaatatcagggtaagttacaaacgtagcagtcagatcatttgtgcaaaaggcaaaaatatgtaattcttcttcatatgtcaatcagttcacacattgtgccagttttaagttaattttcttcttgttcatgacaaatggaggtgattgatcgtcgtttgcccctttgataaaatttgcagggtcgatgagttcataggaaagaatgccgatccattcataacgatgtatggattttcaggttgagaaattgtcatggcctacaagatatctttgttcttactagtatctctctttacaagcttgcagttcatctgagaagggaaacgccgagcaatctcatcccttcccaaagaagatccggtaatcattttcttcccgcagatcttaatatatgttgatgggtcactggaaagaaaatgctgccagatcaacccaagctgattcaaattgttgtactaaacttcacatgtttatatttaaagcatttagaagtttgaaatattgctagttttcccatttttacatctgcactctgcaaaccttgacaattatgaatcagccagtaattcgtgcatcgatttgaactcgtttttcatcagtcattgtaatgaactgactgagggggatgttaatgtctgtgtctgcagaacccggtgggcgtgtttgagctgcttcccgtggatctgcggcggtgggtgctccgccgtccagctcaaggggccgagctgctgctgcggatgcccccggtgctgcgcggggagcgggggctgcggcggcggaccctcgtgtggctgctcatgctcctgcgccggttgctcctcatcttgcgcgtgccctgcctgtgccggctgcggcaccgtgtgctgcggcggcgtccctcgccctcgctgctgcctgtgttcatgaggcgccgaattgagatttttcttcttcattgttcttgctgtgtgcgttgcctgtaggcccagtctcaggctcagctggtgtttttgacgggcatgatcatgtcgtatttctggctcagttggcgctgtggatttgcgttttacttgccacggagatggatcacttgtgctttcgttcttgctatagtctcctacgtt

SEQ ID NO: 61 - DNA, 6003 bp, Triticum aestivum
  source 1..6003: mol_type = genomic DNA
ccacataacaaagcctcgaaaaagggtaatcatttttcatccctcatctgactaacctgaataactttcaatgaaaaaacggtctaatatgatatcttggagctcctgcagcccctgaagctgacttgccacaacaacctcctcatatcgtggagcaaggatgtgctatacctatggatgcacctactgtcgctacaaactcaagtgcaaacgacacatttccttcctgggacgatgttcttacccagccttatagcccaagacatacacaaggtttacaattgtgttaactaccgcatattctgtctatggtcaatatttttacaaggtaccactaacttgtttgctcttgttgaaccgacagatttaggcgctgcaaataatagctctagtagcatagagtgccaatctggaacttgggcaacttctgatgcgattgagcaagaaacttggccaacttccgatgcgattgagcaagagattatacttccagcactgcatggtgagcttctctttgtacatcatacatctatacagtttgttaacatgctggagactgttgtcgacatctatacaattttttaataaattcacaaccctatggattatggtggcaactgttgtgcctatactaagattcatctgattatattagatgtaaactggaatgcatgatatgtgcaaattctattccatttgttaaagtgcctggtatgtgcaaattccattccacatatctggcagatgtgcatgatatgagagtacactaacaaggctagtttaactgccattcattattttttgaaccttttagtttgtggtgtcaatcagtgtgtttttccctttttaactctgttggagccttctgcatttggcttaccttcattctgtccatcaattcttgcaattttgccataatgctcttgatgtctgggtgataaatgatgtaccctgttacaatatgaaaatgtaatgatgtgcagtatttcatattaccctttgatggccttgatattaatcaacatgtttgcctttttggtctatcagttcccagtctaacagttgttttgtacttattctggttaacttgcagccatgccagactttgctcaagtatacaactttcttgggggcgtatttgatccagatacaacaggacatttgcagaagttgagagagatggatcctatagatgctgaaacggtatgccatttgctaatctcccctctataattgattcatctgccctgctataaagctattaatatctgcaagcatgaaggccaggtcattttgtttgcattgtttagtgacatcccagttactcgcaagctgctatgtcagtacctcgattcagtcagtgtgatataaaaacctatgaaactgttccggacaggtggattctggggtgaagtttaattactcattgttgcacattgctccttatcaaggatatttagctgttgtaattcttatatatgttacttatatgttcaccgcaaatgcagatgcttactttgttcattcttttaacagttaaaatgtactgtcagggagtcgcttttattacctaataacttctactccattacaatgatctttttatgggctaatctacaggtactacaactgatgaaaaatctatcggtaaacctgtctggcccggactttgagacacacgtaaggacttaaattattttttcttgcacaccatctgcttaatatttactccgtgatccaacatatttgtctacctactgtaaacaaagtagctgatactactgtagactagaagtagcttgcggtattgtcgaacagttctcgaattgttttccatcccgacatccacattagtgtcacaactatttttaaaaaactaattgttttagcataataattttgcttcatttgtgaatctgccacaacttgtagtactaatccattatatgcgaagattcttttttgtgactgttctgaactgaatcaacacagaaccagttatggtccactccatgttgcacaataatcgaatcggtgctatcctgttatgtaaaataaaagtaaatggcaggatggtgtttcgcttgctaggtgctttgtcaggaatgaagcatatagtatataatattgtgtttcatgagatcatttcatgctccctaatatgttgcgattcatctttcttggaaatccagctgagcatacagcatccctgactgttgcagaggaggatgctctcgtcgcacggtggcggcatgtgctaagccaagcacaagatcgtaggaaatccggggtctactcagacccttcgcattccattcatgtaatctctagtcaccctcttcctgagcacggatccatgcttgcggcattgctaatttcgctaagcgcatggaccgctctgcagggtaaggagcaaatgaggggagaggacagtacattgttgtcagaggtgcgacatgttgcagtatcgcttgttgtcagaggtgcaacatgtttctgcatcaccacgtgcctatgtttgtacatagttcttggagatcttttcgggcttttagatgttggaacttttatctatctgttgatgaggctttgtggcatgtatagtcagggcatggttgcgcatgttgttttggagaggggagaggggaaagtgtttattgcccacgaaggaagaccgtttctgtgtgtaattctttgttttgagctgattttgtttgtaagtccattatcttcatcgtcatatgaggacggatttggactatgtatgcagtgaactcttttcgtctcgtctactatcattgatcatgttttttccgaacgaaaaatgataatcaagtcgtcaatcagctactagcatttgccaaagttgctttgttttttaaccccaaaagaactggacttgattagctgcttcagaaaagaacaaaatgctaacagtttttttatttagagtggagtcaaaggatggagagaagtggtggtcgtggtaggcccgccacgatccagccggcccagcagccttggttaaccctacccactacccaccaccacctacctaccccagtggccagccctatagacgacttcctgtctccttccggcggccacctccgtccatcgctctccgccgccgccgccacccggactctaaatcccttgctcctctccaccggaaaccttgaccggccgaggaggcagcacattcacaccggcggcgtgatctggtctggtctgctagattgatcgttcattcactccattgatgacgctctctgcttccccgcactccccgccgccgtctacctcccggcccccgctcctctcctggcccagctagccagccggcgcgagcgagcgagctccccgtccaccgtccagctattagccaagctccttcgaggccggcaatggcggcgcccaggcccaagtccccgctcgacccctgcggccgccaccggctgcagctcgccgtcgacgcgctccaccgccagatcagcttcctcgaggtaaccaagctcctgaccttaatcgctcttcctggttggctcccgcactactgccatgataaatgatgccctgctgttactcagttattccgcaagaaaaattacagtactaattagcagctgagcatgtacaaatgatcgccatttctttataggtatgagtactctattataggtatctaaagtccgctaccaacacacagtttggttgtcacttctaagtcagttccaagtgttcttttctactactactagattctagagtaagtgttaaaatatccccccagtttagcagtaagcatacaaaaatgatcagcatttctttataggttggttgccaacacacggtttggttgtcacttctcagtcaattccaggtgttcctttctagtactccctccgttccaaattagtcgtcgtggttttagttcaaattaaaactaaaaccacgacgagtaatttggaacggtgggagtagtagtagattccaagtgttgttaaaatatcccagacagcagcctgaaaagttgacacatgccctctcctccttgtactgtattgctagtagctatactagctagtagctactgctttttttcaataaaaaaaatcatttctatggaattcaagaaactgttcttctgtgccagggggagatcagttccattgaagggctccatgctgcctccatatgctgcaaagagtaagccgctgtcttgttcaattcttatactatcaatttcctttttgaaatttaagaggtaacttagggagcgaaaacaagaaatagttctccggctccaagaatggaaaacaatccatggcaagtgagcccatgtttccatagagttgtacagatgcgtgcaagacatcaacatggatgtgtgaaggcaacacagattttagttaaaacctctaaatagaagtttcgcgttttagccaagaaatgtcgctctcagtggagatttatacaataatactccctccgtcccaaaattcttgtcttagatttgtctaaatacagatgtatcaagtcacgttttagtattagatacatccgtatctagactaatctaagacaagaattttgggacggagggagtagattactatctacgaccatgggcttccacaccaatgattgaaaatagtatacacttacaactgaacattatgcaaatccggtagcaatagtttgggagatttggttagttgaaatttgattaggatgattgattgtatttaaatacgtgaatatgttttggaattggacttaaattacatagggttgcggtgtgcttttattttcatctccagtcttggtgccacttattactttattcttcttcataaatgaatatcagtgtaagctgcatacgtagcagtcagatcatgcgtgcaaaaggcaaaaatatgtaattcttcttcatgtgtcaatcagttcacacaattgtgccaattttaagttaattttcttcttgttcatgacatatggaggtgattgatcgtcgtttgcccctttgataaaatttgcagggtcgatgagttcataggaaagaatgccgatccattcatcacgatgtatggattttcaggttgagaaattgtcttggcctacaagatatctttgttcttactagtatctctctttacaagcttgcagttcatctgagaagggaaatgctgatcaatctcatcgcttcccaaagaagattcggtaatcatttcctccctgcagatcttaatatatgttgatggttgtcttgaaagaaaatgacgccggatcaacccaagctgatgcaaattgttatcctaaactgcacttgaagcatttaaaagtttgaaatattgctaattttccggtttttacatctgcactctacaaaccttaacaattatttgtcagccagtaatgcatgcatcgatttgaactcgtttttagttagccgttgtagtgaactgaccgagggggatgttaatgtttgtgtctgcagaacccggtgggcgtgtttgagctgcttcccgtggatctgcggcggcgggtgctccgccgtccagctcaaggggccgagctgctgctgcggatgcccccgttgctgcgtggggagcgggggctgcggcggcggaccctcgtgtggctgctcatgctcctgcgccggttgctcctcctcttgcgcgtgccctgcctgcgccggctgcggcgccgcgtgctgcggcggtgcccctcgccctcgctgctgcctgtgttcatgaggcgccctattgagatttttcctcttctttgttcttgctgtgtgcgttgcctgtaggctcaggctcggctggtgtttttgacgggcatgatgatgatgatgtatttctggctcagttggcgctgtggatttgcctggttttacttgccacagcgatggatcccttgtgctttcgtccttgctatagtctcctatgttgtaattaaaggtttggagataactgcagaatgaacggttcttctcac

SEQ ID NO: 62 - DNA, 1012 bp, Triticum aestivum
  source 1..1012: mol_type = unassigned DNA
accccagtctccagccctacagaccgacgacttcctgtctcctcctcccggcggccacctccgtccatcgctctccccccctctctccgccgccgccgccgggcctctgaatccccttgctcctccttccgaggaggcagcacatcagcatcagcggcgtgatctgatccggtctgctagactgatcgctcattcactccattgatgacgctctctgcttccccgcactccccgccgccgtccacctcccgccccccgctcctctcctggcccagctagccagccggcgcgagcgagccctccatccaccaccgtccagctagccaggctcctccgaggccggcaatggcggcgcccaggcccaagtccccgctcgacccctgcggccgccgccggctgcagctcgccgtcgacgcgctccaccgccagatcagcttcctcgagggggagatcagttccattgaagggctccatgctgcctccatatgctgcaaagaggtcgatgagttcataggaaagaatgccgatccattcataacgatttcatctgagaaggggaacgccgatcaatctcatcgctccccaaagaagattcgaacccggtgggcgtgtttgagctgctcctcctcttgcgcgtgccctgcctgtgccggctgcggccccgcgtgctgcggcggtgtccctcgccctcgctgctgcctgtgttcatgatcatgaggcgccgtattgagatttttcttcttcttcttcgttcttgctgtgtgcgttgtgcgttgcctgtaggctcaggctcggctggtgtttttgacgggcatgacgatgatgatgtatttctggctcagttggcgctgtggatttgcctggttttacttgccgcagagaaggatcgcttgggccttcgtccttcctatagtctcctacgttgtaattaaagctttggagataactgcagaatgaacgggttttctcactgatgagtgatgatggtcagggtgtatatatgcccagttag

SEQ ID NO: 63 - DNA, 1159 bp, Triticum aestivum
  source 1..1159: mol_type = unassigned DNA
accccagtctccagccctacagaccgacgacttcctgtctcctcctcccggcggccacctccgtccatcgctctccccccctctctccgccgccgccgccgggcctctgaatccccttgctcctccttccgaggaggcagcacatcagcatcagcggcgtgatctgatccggtctgctagactgatcgctcattcactccattgatgacgctctctgcttccccgcactccccgccgccgtccacctcccgccccccgctcctctcctggcccagctagccagccggcgcgagcgagccctccatccaccaccgtccagctagccaggctcctccgaggccggcaatggcggcgcccaggcccaagtccccgctcgacccctgcggccgccgccggctgcagctcgccgtcgacgcgctccaccgccagatcagcttcctcgagggggagatcagttccattgaagggctccatgctgcctccatatgctgcaaagaggtcgatgagttcataggaaagaatgccgatccattcataacgatttcatctgagaaggggaacgccgatcaatctcatcgctccccaaagaagattcgaacccggtgggcgtgtttgagctgcttcccgtggatctgcggcggcgggtgctctgccgtccagctcaaggggccgagctgctgctgcggatgcccccgctgctgcgcggggagcgggggctgcggcggcggcgggccctcgtgtggctgctcgtgctcctgcgccggctgctcctcctcttgcgcgtgccctgcctgtgccggctgcggccccgcgtgctgcggcggtgtccctcgccctcgctgctgcctgtgttcatgatcatgaggcgccgtattgagatttttcttcttcttcttcgttcttgctgtgtgcgttgtgcgttgcctgtaggctcaggctcggctggtgtttttgacgggcatgacgatgatgatgtatttctggctcagttggcgctgtggatttgcctggttttacttgccgcagagaaggatcgcttgggccttcgtccttcctatagtctcctacgttgtaattaaagctttggagataactgcagaatgaacgggttttctcactgatgagtgatgatggtcagggtgtatatatgcccagttag

SEQ ID NO: 64 - DNA, 1015 bp, Triticum aestivum
  source 1..1015: mol_type = unassigned DNA
tggccagccctatagacgacttcctgtctccttccggcggccacctccgtccgtctctctccccctctccgccgccgccgccgccgcccggcctctaaacccccttggtcctccccccagcccccacaccggcggcgtgatctgctctggtctgctagattgatcgcccattcattccattgatgacgctctctggctccccgcactccccgccgccgtccaccagccgccccccgctcctctcctggcctagctagccagccagccggcgcgagcgacccctccatccaccaccgtccagctagctagctagctccttcgaggccggcaatggcggcgcccaggcccaagtccccgctcgacccctgcggccgccaccgcctgcagctcgccgtcgacgcgctccaccgccagatcagcttcctcgagggggagatcaattccattgaagggctccatgctgcctccatatgctgcaaagaggtcgatgagttcataggaaagaatgccgatccattcataacgatttcatctgagaagggaaacgccgagcaatctcatcccttcccaaagaagatccgaacccggtgggcgtgtttgagctgcttcccgtggatctgcggcggtgggtgctccgccgtccagctcaaggggccgagctgctgctgcggatgcccccggtgctgcgcggggagcgggggctgcggcggcggaccctcgtgtggctgctcatgctcctgcgccggttgctcctcatcttgcgcgtgccctgcctgtgccggctgcggcaccgtgtgctgcggcggcgtccctcgccctcgctgctgcctgtgttcatgaggcgccgaattgagatttttcttcttcattgttcttgctgtgtgcgttgcctgtaggcccagtctcaggctcagctggtgtttttgacgggcatgatcatgtcgtatttctggctcagttggcgctgtggatttgcgttttacttgccacggagatggatcacttgtgctttcgt

SEQ ID NO: 65 - DNA, 1102 bp, Triticum aestivum
  source 1..1102: mol_type = unassigned DNA
cccagtggccagccctatagacgacttcctgtctccttccggcggccacctccgtccatcgctctccgccgccgccgccacccggactctaaatcccttgctcctctccaccggaaaccttgaccggccgaggaggcagcacattcacaccggcggcgtgatctggtctggtctgctagattgatcgttcattcactccattgatgacgctctctgcttccccgcactccccgccgccgtctacctcccggcccccgctcctctcctggcccagctagccagccggcgcgagcgagcgagctccccgtccaccgtccagctattagccaagctccttcgaggccggcaatggcggcgcccaggcccaagtccccgctcgacccctgcggccgccaccggctgcagctcgccgtcgacgcgctccaccgccagatcagcttcctcgagggggagatcagttccattgaagggctccatgctgcctccatatgctgcaaagaggtcgatgagttcataggaaagaatgccgatccattcatcacgatttcatctgagaagggaaatgctgatcaatctcatcgcttcccaaagaagattcgaacccggtgggcgtgtttgagctgcttcccgtggatctgcggcggcgggtgctccgccgtccagctcaaggggccgagctgctgctgcggatgcccccgttgctgcgtggggagcgggggctgcggcggcggaccctcgtgtggctgctcatgctcctgcgccggttgctcctcctcttgcgcgtgccctgcctgcgccggctgcggcgccgcgtgctgcggcggtgcccctcgccctcgctgctgcctgtgttcatgaggcgccctattgagatttttcctcttctttgttcttgctgtgtgcgttgcctgtaggctcaggctcggctggtgtttttgacgggcatgatgatgatgatgtatttctggctcagttggcgctgtggatttgcctggttttacttgccacagcgatggatcccttgtgctttcgtccttgctatagtctcctatgttgtaattaaaggtttggagataactgcagaatgaacggttcttctcac

SEQ ID NO: 66 - DNA, 366 bp, Triticum aestivum
  source 1..366: mol_type = unassigned DNA
atggcggcgcccaggcccaagtccccgctcgacccctgcggccgccgccggctgcagctcgccgtcgacgcgctccaccgccagatcagcttcctcgagggggagatcagttccattgaagggctccatgctgcctccatatgctgcaaagaggtcgatgagttcataggaaagaatgccgatccattcataacgatttcatctgagaaggggaacgccgatcaatctcatcgctccccaaagaagattcgaacccggtgggcgtgtttgagctgctcctcctcttgcgcgtgccctgcctgtgccggctgcggccccgcgtgctgcggcggtgtccctcgccctcgctgctgcctgtgttcatga

SEQ ID NO: 67 - DNA, 513 bp, Triticum aestivum
  source 1..513: mol_type = unassigned DNA
atggcggcgcccaggcccaagtccccgctcgacccctgcggccgccgccggctgcagctcgccgtcgacgcgctccaccgccagatcagcttcctcgagggggagatcagttccattgaagggctccatgctgcctccatatgctgcaaagaggtcgatgagttcataggaaagaatgccgatccattcataacgatttcatctgagaaggggaacgccgatcaatctcatcgctccccaaagaagattcgaacccggtgggcgtgtttgagctgcttcccgtggatctgcggcggcgggtgctctgccgtccagctcaaggggccgagctgctgctgcggatgcccccgctgctgcgcggggagcgggggctgcggcggcggcgggccctcgtgtggctgctcgtgctcctgcgccggctgctcctcctcttgcgcgtgccctgcctgtgccggctgcggccccgcgtgctgcggcggtgtccctcgccctcgctgctgcctgtgttcatga

SEQ ID NO: 68 - DNA, 510 bp, Triticum aestivum
  source 1..510: mol_type = unassigned DNA
atggcggcgcccaggcccaagtccccgctcgacccctgcggccgccaccgcctgcagctcgccgtcgacgcgctccaccgccagatcagcttcctcgagggggagatcaattccattgaagggctccatgctgcctccatatgctgcaaagaggtcgatgagttcataggaaagaatgccgatccattcataacgatttcatctgagaagggaaacgccgagcaatctcatcccttcccaaagaagatccgaacccggtgggcgtgtttgagctgcttcccgtggatctgcggcggtgggtgctccgccgtccagctcaaggggccgagctgctgctgcggatgcccccggtgctgcgcggggagcgggggctgcggcggcggaccctcgtgtggctgctcatgctcctgcgccggttgctcctcatcttgcgcgtgccctgcctgtgccggctgcggcaccgtgtgctgcggcggcgtccctcgccctcgctgctgcctgtgttcatga

SEQ ID NO: 69 - DNA, 510 bp, Triticum aestivum
  source 1..510: mol_type = unassigned DNA
atggcggcgcccaggcccaagtccccgctcgacccctgcggccgccaccggctgcagctcgccgtcgacgcgctccaccgccagatcagcttcctcgagggggagatcagttccattgaagggctccatgctgcctccatatgctgcaaagaggtcgatgagttcataggaaagaatgccgatccattcatcacgatttcatctgagaagggaaatgctgatcaatctcatcgcttcccaaagaagattcgaacccggtgggcgtgtttgagctgcttcccgtggatctgcggcggcgggtgctccgccgtccagctcaaggggccgagctgctgctgcggatgcccccgttgctgcgtggggagcgggggctgcggcggcggaccctcgtgtggctgctcatgctcctgcgccggttgctcctcctcttgcgcgtgccctgcctgcgccggctgcggcgccgcgtgctgcggcggtgcccctcgccctcgctgctgcctgtgttcatga

SEQ ID NO: 70 - DNA, 23 bp, synthetic construct
  source 1..23: mol_type = other DNA; note = TaGS target sequence
aagtccccgctcgacccctgcgg

SEQ ID NO: 71 - DNA, 24 bp, synthetic construct
  source 1..24: mol_type = other DNA; note = TaGS 1F
ggcgaagtccccgctcgacccctg

SEQ ID NO: 72 - DNA, 24 bp, synthetic construct
  source 1..24: mol_type = other DNA; note = TaGS 1R
aaaccaggggtcgagcggggactt

SEQ ID NO: 73 - DNA, 13 bp, synthetic construct
  source 1..13: mol_type = other DNA; note = TaGS 4A mutation in E5
cccgctcgacccc

SEQ ID NO: 74 - DNA, 708 bp, Glycine max
  source 1..708: mol_type = other DNA
atggccacttctcccaccaccaccaccaccaccaccgttcgttcttcttccgtcccttctctgcctccaccctcccccaagtccccgccggagtacccagatttgtatggcaagcgccgtgaaaccgccagggttcacatgctcgagagggaaatcacttttctcgagcaggaagaattaaaatctgttgaaggccttcaagcagcttcaagatgctgcaaagagattgctgattatgtgatggcaaacgcagatcctctgttaccttcaaccaagaagaaccgtcggtcatgtggcttctggaagtggctctgtggcatgccctgttttaacctctcttggatctgttgctgctgttgctgctgtgaagggttatctttacaactaaaattgccacgctgctgttgtgactgcaaaccatgcagttgcagttgcagctgtcttccatccatcaaatgctgctccttaccaaagtggagctgttgttgctcttgccccaaatcaaattgctgtaaagagggttgcggttttggaaattgttgtactttcccacgtagttgcaattttgggtgcccaacttgtccatcttgcccctcttgctgcagttgcaaatgcacctgcacttgctcttgcccaagctgtccaaaggtaagcccatgttgctgttgtacaaagtcatgttggagaccttgttgtttttgttgctag

SEQ ID NO: 75 - protein, 235 aa, Glycine max
MATSPTTTTTTTVRSSSVPSLPPPSPKSPPEYPDLYGKRRETARVHMLEREITFLEQEELKSVEGLQAASRCCKEIADYVMANADPLLPSTKKNRRSCGFWKWLCGMPCFNLSWICCCCCCCEGLSLQLKLPRCCCDCKPCSCSCSCLPSIKCCSLPKWSCCCSCPKSNCCKEGCGFGNCCTFPRSCNFGCPTCPSCPSCCSCKCTCTCSCPSCPKVSPCCCCTKSCWRPCCFCC

SEQ ID NO: 76 - DNA, 687 bp, Glycine max
  source 1..687: mol_type = other DNA
atggccactactcccaccaccgttcgttcttcttccgtcccttctctgcctccaccctctcctaagtccccgccggagtacccagatttgtatggcaagcgccgtgaaaccgccagggttcacatgctcgagagggaaatcacttttctcgaggaagaattaaaatctgttgagggccttcaaccagcttcaagatgctgcaaagagattgctgattatgtgatggcaaacgcagatcccctgttaccttcaaccaagaagaaccgccggtcatgtcgcttctggaagtggctctgtggcatgccctgttttaacctctcttggatctgctgttgttgctgctgtgaagggttatctttacaactaaaattgccacgctgctgttgtgactgcaaaccatgcagttgcagttgcagttgtcttccacccatcaaatgctgctccttaccaaagtggagctgttgttgctcttgccccaaatcaaattgctgtaaagagggttgtggttttggaaattgttgtactttcccacgtagttgcaattttgggtgtccaacttgtccatcttgcccctcttgctgcagttgcaaatgcacctgcacttgctcttgcccaagctgtccgaaggtaagcccatgttgctgttgtacaaagtcatgctggagaccatgttgtttttgttgctag

SEQ ID NO: 77 - protein, 228 aa, Glycine max
MATTPTTVRSSSVPSLPPPSPKSPPEYPDLYGKRRETARVHMLEREITFLEEELKSVEGLQPASRCCKEIADYVMANADPLLPSTKKNRRSCRFWKWLCGMPCFNLSWICCCCCCEGLSLQLKLPRCCCDCKPCSCSCSCLPPIKCCSLPKWSCCCSCPKSNCCKEGCGFGNCCTFPRSCNFGCPTCPSCPSCCSCKCTCTCSCPSCPKVSPCCCCTKSCWRPCCFCC

SEQ ID NO: 78 - DNA, 654 bp, Glycine max
  source 1..654: mol_type = other DNA
atgagcacttccacaagggcaacttccaacatccttctctctctgccactgccctctcctaaggcggcgcctccttcgtgcccagatttgtatggaaagcgtcgcgaaatggccaagattcagatgctggaaagagagataagtttcctcgaggaagagttaaaatcttctgaaggccttcaaccagcttcaagatgctgcaaagagattgccgattttgtgatggcaaactcagatcctctgttacctacgagcaagaagaaccaccggtcatgtagcttctggaagtggctgtgtgacatcccttgctttaacttgtcttggatctgccattggtgctgtgatgggtgctttgaacatcaaaacatgtcaagttgttgttcccactgcatgccatgcaattgctgttctagttgtcttccatctaccaattgctcttgctgccctaatggaatatcacattgctgcaaagatagttgtggttgcaaaatttgttgcactctcccaagttgcaatattgggtggccttttccctcttgctgcatttgcaaatgctcttgctcttgctcttggtcaggcccaaaaaaatgtcccaaggttcgtccatgttgctgttgtacaaattcctgttggaacccctgttcatgtttctag

SEQ ID NO: 79 - protein, 217 aa, Glycine max
MSTSTRATSNILLSLPLPSPKAAPPSCPDLYGKRREMAKIQMLEREISFLEEELKSSEGLQPASRCCKEIADFVMANSDPLLPTSKKNHRSCSFWKWLCDIPCFNLSWICHWCCDGCFEHQNMSSCCSHCMPCNCCSSCLPSTNCSCCPNGISHCCKDSCGCKICCTLPSCNIGWPFPSCCICKCSCSCSWSGPKKCPKVRPCCCCTNSCWNPCSCF

SEQ ID NO: 80 - DNA, 690 bp, unidentified
  source 1..690: mol_type = other DNA
atggccactactcccaccaccgttcgttcttcttccgtcccttctctgcctccaccctctcctaagtccccgccggagtacccagatttgtatggcaagcgccgtgaaaccgccagggttcacatgctcgagagggaaatcacttttctcgagcaggaagaattaaaatctgttgagggccttcaaccagcttcaagatgctgcaaagagattgctgattatgtgatggcaaacgcagatcccctgttaccttcaaccaagaagaaccgccggtcatgtcgcttctggaagtggctctgtggcatgccctgttttaacctctcttggatctgctgttgttgctgctgtgaagggttatctttacaactaaaattgccacgctgctgttgtgactgcaaaccatgcagttgcagttgcagttgtcttccacccatcaaatgctgctccttaccaaagtggagctgttgttgctcttgccccaaatcaaattggtgtaaagagggttgtggttttggaaattgttgtactttcccacgtagttgcaattttgggtgtccaacttgtccatcttgcccctcttgctgcagttgcaaatgcacctgcacttgctcttgcccaagctgtccgaaggtaagcccatgttgctgttgtacaaagtcatgctggagaccatgttgtttttgttgctag

SEQ ID NO: 81 - protein, 229 aa, unidentified
MATTPTTVRSSSVPSLPPPSPKSPPEYPDLYGKRRETARVHMLEREITFLEQEELKSVEGLQPASRCCKEIADYVMANADPLLPSTKKNRRSCRFWKWLCGMPCFNLSWICCCCCCEGLSLQLKLPRCCCDCKPCSCSCSCLPPIKCCSLPKWSCCCSCPKSNWCKEGCGFGNCCTFPRSCNFGCPTCPSCPSCCSCKCTCTCSCPSCPKVSPCCCCTKSCWRPCCFCC

SEQ ID NO: 82 - DNA, 705 bp, unidentified
  source 1..705: mol_type = other DNA
atggccacttctcccaccaccaccaccaccaccaccgttcgttcttcttccgtcccttctctgcctccaccctcccccaagtccccgccggagtacccagatttgtatggcaagcgccgtgaaaccgccagggttcacatgctcgagagggaaatcacttttctcgaggaagaattaaaatctgttgaaggccttcaagcagcttcaagatgctgcaaagagattgctgattatgtgatggcaaacgcagatcctctgttaccttcaaccaagaagaaccgtcggtcatgtggcttctggaagtggctctgtggcatgccctgttttaacctctcttggatctgttgctgctgttgctgctgtgaagggttatctttacaactaaaattgccacgctgctgttgtgactgcaaaccatgcagttgcagttgcagctgtcttccatccatcaaatgctgctccttaccaaagtggagctgttgttgctcttgccccaaatcaaattgctgtaaagagggttgcggttttggaaattgttgtactttcccacgtagttgcaattttgggtgcccaacttgtccatcttgcccctcttgctgcagttgcaaatgcacctgcacttgctcttgcccaagctgtccaaaggtaagcccatgttgctgttgtacaaagtcatgttggagaccttgttgtttttgttgctag

SEQ ID NO: 83 - protein, 234 aa, unidentified
MATSPTTTTTTTVRSSSVPSLPPPSPKSPPEYPDLYGKRRETARVHMLEREITFLEEELKSVEGLQAASRCCKEIADYVMANADPLLPSTKKNRRSCGFWKWLCGMPCFNLSWICCCCCCCEGLSLQLKLPRCCCDCKPCSCSCSCLPSIKCCSLPKWSCCCSCPKSNCCKEGCGFGNCCTFPRSCNFGCPTCPSCPSCCSCKCTCTCSCPSCPKVSPCCCCTKSCWRPCCFCC

SEQ ID NO: 84 - DNA, 654 bp, unidentified; identical to SEQ ID NO: 78
  source 1..654: mol_type = other DNA

SEQ ID NO: 85 - protein, 217 aa, synthetic construct; identical to SEQ ID NO: 79

SEQ ID NO: 86 - protein, 67 aa, Setaria italica
LQLAVDALHREIGFLEGEISSIDGVHAASRCCKEVDEFVGRNPDPFITIQPEKRSNEQSQQFLKKFR

SEQ ID NO: 87 - protein, 67 aa, Oryza sativa
LQLAVDALHREIGFLEGEINSIEGIHAASRCCREVDEFIGRTPDPFITISSEKRSHDHSHHFLKKFR

SEQ ID NO: 88 - protein, 67 aa, Zea mays
LQLAVDALHREIGFLEGEISSIEGVHAASRCCKEVDEFVGRNPDPFLTIQQERGSHDQSQQFLKKFR

SEQ ID NO: 89 - protein, 67 aa, Triticum aestivum
LQLAVDALHRQISFLEGEISSIEGLHAASICCKEVDEFIGKNADPFITISSEKGNADQSHRSPKKIR

SEQ ID NO: 90 - protein, 67 aa, Triticum aestivum
LQLAVDALHRQISFLEGEINSIEGLHAASICCKEVDEFIGKNADPFITISSEKGNAEQSHPFPKKIR

SEQ ID NO: 91 - protein, 67 aa, Triticum aestivum
LQLAVDALHRQISFLEGEISSIEGLHAASICCKEVDEFIGKNADPFITISSEKGNADQSHRFPKKIR

SEQ ID NO: 92 - protein, 64 aa, Glycine sp.
EMAKIQMLEREISFLEEELKSSEGLQPASRCCKEIADFVMANSDPLLPTSKKNHRSCSFWKWLC

SEQ ID NO: 93 - protein, 65 aa, Glycine max
ETARVHMLEREITFLEQEELKSVEGLQAASRCCKEIADYVMANADPLLPSTKKNRRSCGFWKWLC

SEQ ID NO: 94 - protein, 64 aa, Glycine max
ETARVHMLEREITFLEEELKSVEGLQPASRCCKEIADYVMANADPLLPSTKKNRRSCRFWKWLC

SEQ ID NO: 95 - protein, 64 aa, Glycine sp.
ETARVHMLEREITFLEEELKSVEGLQAASRCCKEIADYVMANADPLLPSTKKNRRSCGFWKWLC

SEQ ID NO: 96 - protein, 65 aa, Glycine sp.
ETARVHMLEREITFLEQEELKSVEGLQPASRCCKEIADYVMANADPLLPSTKKNRRSCRFWKWLC

(19) United States
(12) Patent Application Publication
(10) Pub. No.: US 2026/0049328 A1
(43) Pub. Date: Feb. 19, 2026

(54) PLANT SALINE-ALKALI-TOLERANT GENE AND USE THEREOF

(71) Applicants: INSTITUTE OF GENETICS AND DEVELOPMENTAL BIOLOGY, CHINESE ACADEMY OF SCIENCES, Beijing (CN); HUAZHONG AGRICULTURAL UNIVERSITY, Wuhan, Hubei (CN); PEKING UNIVERSITY INSTITUTE OF ADVANCED AGRICULTURAL SCIENCES, Weifang, Shandong (CN)

(72) Inventors: Qi XIE, Beijing (CN); Feifei YU, Beijing (CN); Huili ZHANG, Beijing (CN); Yidan OUYANG, Wuhan, Hubei (CN); Qifa ZHANG, Wuhan, Hubei (CN); Huawei ZHANG, Weifang, Shandong (CN); Wenjing SUN, Weifang, Shandong (CN); Shengyuan SUN, Yangzhou, Jiangsu (CN); Xu LI, Wuhan, Hubei (CN); Yaoyao ZHU, Weifang, Shandong (CN)

(21) Appl. No.: 19/103,381

(22) PCT Filed: Mar. 15, 2023
(86) PCT No.: PCT/CN2023/081663

§ 371 Date: Feb. 12, 2025

(30) Foreign Application Priority Data
Aug. 12, 2022 (CN) 202210968618.1
Sep. 29, 2022 (CN) 202211198322.2

(51) Int. Cl. C12N 15/82; C07K 14/415; C12N 9/22; C12N 15/11

(52) U.S. Cl. CPC C12N 15/8273; C07K 14/415; C12N 9/226; C12N 15/11; C12N 15/8205; C12N 15/8213; C12N 2310/20

(57) ABSTRACT

The present invention relates to a plant saline-alkali tolerance related gene AT1 and its homologous genes and their use in producing saline-alkali-tolerant plants. The present invention also relates to a method for producing saline-alkali-tolerant plants, resultant saline-alkali-tolerant plants and plant materials thereof. When in a plant all alleles encoding a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15 have reduced expression levels or are not expressed, the saline-alkali tolerance of the plant is improved; when in a plant the expression level of a gene encoding a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15 is increased, the salt-alkali sensitivity of the plant is improved.

### PRIORITY INFORMATION

The present application claims the priority of Chinese patent application No. 202210968618.1 filed on Aug. 12, 2022 and Chinese patent application No. 202211198322.2 filed on Sep. 29, 2022, both of which are incorporated herein by reference in their entirety.

### TECHNICAL FIELD

The present invention relates to a plant saline-alkali-tolerance-related gene AT1 and homologous gene thereof, and their use in producing saline-alkali-tolerant plants. The present invention also relates to a method for producing saline-alkali-tolerant plants, resultant saline-alkali-tolerant plants and plant materials thereof.

### BACKGROUND ART

Food security is affected by the potential negative impact of global population growth and climate change on agricultural production. Drought climate, rising groundwater levels, and low-lying terrain without drainage outlets are all causes of land saline-alkalization. At the same time, due to the large-scale application of chemical fertilizers, 50% of the fertile land may become saline-alkali land in the near future, which will seriously threaten food production (A. Kumar, S. Singh, A. K. Gaurav, S. Srivastava, J. P. Verma, Plant growth-promoting bacteria: Biological tools for the mitigation of salinity stress in plants. Front. Microbiol. 11, 1216 (2020). doi:10.3389/fmicb.2020.01216). Soil salinization and secondary saline-alkalization are global ecological and resource problems and are one of the important abiotic stress factors that cause crop yield reduction (Yamaguchi, T. and Blumwald, E. Developing salt-tolerant crop plants: challenges and opportunities. (2005). Trends in Plant Science 10:615-620).

According to the survey data of the Food and Agriculture Organization of the United Nations (FAO) in 2015, currently, about 20% of the cultivated land and 50% of the irrigated land in the world are affected by salinization to varying degrees. More than 1 billion hectares of land are affected by salinization, and about 60% of the estimated area is classified as alkaline land (i.e., land with high pH due to high level of alkali mainly composed of NaHCO₃ and Na₂CO₃). About 25-33% of the world's irrigated land is affected by secondary salinity (B. P. Singh, A. L. Cowie, K. Y. and Chan, Soil health and climate change. New York: Springer-Verlag Berlin Heidelberg 29, (2011). doi:10.1007/978-3-642-20256-8). Since it is difficult for ordinary crops to survive on saline-alkali land, saline-alkali land is difficult to become arable land for crop cultivation unless it undergoes very complex and cumbersome desalination treatment.

Salinized soil generally refers to a type of soil affected by salt and alkali, including saline soil and alkaline soil. Saline soil refers to soil with a soluble salt content of more than 2% %, while alkaline soil refers to soil with a proportion of exchangeable sodium ions to soluble cations (ESP) greater than 20% and a pH value greater than 8.0 (Yang Jinsong. (2008). The development and prospects of saline soil research in China. Acta Pedologica Sinica, 45:837-845). In areas where saline-alkali land is present, crops are harmed by saline-alkaline stress to varying degrees throughout the growth period. In particular, in spring, the earth's surface will be subjected to “accumulation of salt”. This is because the earth's surface water evaporates strongly, and the salt in the groundwater accumulates in the soil surface as the capillary water rises. Spring is the season for sowing crops, and saline-alkaline stress is extremely harmful to the germination period of crops. In order to solve the problem of how to turn saline-alkali land into fertile farmland, a variety of technologies have been applied to improve soil saline-alkalization. For example, chemical and physical methods are used to change the degree of saline-alkalization, or farming measures are used to improve saline-alkali land. Although both of the two methods can improve the physical and chemical properties of the soil and the soil texture, they take a long time and are costly. Therefore, the problem of effective utilization of saline-alkali land can be fundamentally solved by analyzing the molecular mechanism of plant response to saline-alkaline stress and cultivating saline-alkali-tolerant varieties through molecular biological techniques (Glenn, E. P., Brown, J. J. and Blumwald E. (1999). Salt tolerance and crop potential of halophytes. Critical Reviews in Plant Sciences 18:227-255; Qian Qian, Qi Xiaoquan, Lin Rongcheng, Yang Shuhua, Dong Aiwu, Zuo Jianru, Chen Fan, Xiao Langtao, Gu Hongya, Chen Zhiduan, Bai Yongfei, Wang Tai. (2019). Important research progress in several fields of Chinese plant science in 2018. Acta Botanica Sinica 54:405-440).

In addition, by analyzing the papers published in the Web of Science (http://www.webofscience.com/) in the past 20 years, there are as many as 22,614 papers related to salt tolerance, while there are only 457 papers related to alkalinity tolerance. Due to insufficient understanding of alkaline stress, the cultivation of alkali-tolerant/saline-alkali-tolerant crops and the development of technology of increasing saline-alkali land crop yield are limited. In fact, salt-tolerance and alkali-tolerance are two different traits for plants. According to FAO statistics in 2015, among the more than 1 million hectares of saline-alkali land surveyed, 60% can actually be defined as land alkalization caused by Na₂CO₃ or NaHCO₃. Unlike neutral salinity (pH of about 7) which only has ion toxicity, alkaline salinized soil has a high pH value, which reduces the plant's absorption rate of essential nutrients and the excretion rate of sodium ions (Na⁺). Compared with salinity alone, alkaline salinized soil has more negative effects on plant growth by inducing high cellular oxidative stress (M. Javid, R. Ford, M. E. Nicolas, Tolerance responses of Brassica juncea to salinity, alkalinity and alkaline salinity. Funct. Plant Biol. 39, 699-707 (2012). doi:10.1071/FP12109).

Therefore, soil saline-alkalization may become a global problem affecting plant growth and crop production. Using these saline-alkali lands for crop production will help to meet future food needs. Therefore, studying the mechanism of crop tolerance to alkali/salt-alkali will help to manage alkali/saline-alkali lands, and cultivating new alkali/saline-alkali-tolerant crop varieties can also expand the cultivated land area, and is an effective measure to increase the total crop yield. Studying the mechanism of crop tolerance to salinity and alkalinity and cultivating more saline-alkali-tolerant crops may be a priority for future agriculture.

### SUMMARY OF INVENTION

The present invention has an aim to study the mechanism of plant alkali/saline-alkali tolerance at the genetic level, develop methods for producing saline-alkali-tolerant plants, and produce plants with higher saline-alkali tolerance.

In the study, the present inventors have identified and demonstrated that the natural allele of AT1, an atypical G protein γ subunit (Gγ subunit), contributes to the alkali tolerance of five different monocotyledonous crops (sorghum, millet, rice, maize and wheat). The N-terminal domain of AT1 and its homologues plays a negative regulatory role in alkaline stress tolerance. Crops containing C-terminal truncated AT1 proteins are highly sensitive to alkaline stress. This may be due to the inhibitory effect of the C-terminal domain, which is essential for protein degradation in its rice homologues (S. Sun, L. Wang, H. Mao, L. Shao, X. Li, J. Xiao et al., A G-protein pathway determines grain size in 45 rice. Nat. Commun. 9, 851 (2018). doi:10.1038/s41467-018-03141-y, W. Yang, K. Wu, B. Wang, H. Liu, S. Guo, X. Guo et al., The RING E3 ligase CLG1 targets GS3 for degradation via the endosome pathway to determine grain size in rice. Mol. Plant 14, 1699-1713 (2021). doi:10.1016/j.molp.2021.06.027). Therefore, overexpression of the entire AT1 protein produces a higher amount of the protein, resulting in a higher sensitivity to alkaline stress, while overexpression of the C-terminal truncated protein results in a higher sensitivity to alkaline stress. In contrast, high tolerance to alkalinity/salinity is observed in all five crops due to the knockout of the gene/natural variation with a non-functional allele, which results in a lack of the N-terminal GGL domain. On this basis, the present inventors have completed the present invention.

In a first aspect, the present invention provides a use of AT1 or homologous gene thereof in regulating saline-alkali tolerance in a plant or producing a plant with saline-alkali tolerance or saline-alkali sensitivity.

In some embodiments, the AT1 or homologous gene thereof comprises a GGL domain or a GGL-like domain, and the GGL domain or the GGL-like domain comprises an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15.

In some embodiments, the GGL domain or the GGL-like domain comprises an amino acid sequence as set forth in any one of SEQ ID NOs: 15 and 86-96.

In some embodiments, increasing the expression level (e.g., overexpression) of AT1 or homologous gene thereof, or expressing or overexpressing a C-terminal truncated protein encoded by a variant of AT1 or homologous gene thereof in a plant can improve the saline-alkali sensitivity of the plant.

In some embodiments, reducing the expression level of all alleles of AT1 or homologous gene thereof (e.g., by gene editing, targeted mutagenesis, chemical induction, radiation induction, natural mutation, RNAi, or addition of a substance capable of inhibiting target gene expression) or making the all alleles non-expressed in a plant can improve the saline-alkali tolerance of the plant.

The saline-alkali-tolerant plant of the present invention grows better than a wild-type plant under saline-alkali conditions, wherein the saline-alkali conditions comprise growth conditions at pH >7.5 and Na⁺ concentration >75 mM or growth conditions at pH >8.0 and Na⁺ concentration >50 mM.

In some embodiments, knocking out the N-terminal GGL domain or GGL-like domain of all alleles of AT1 or homologous gene thereof or knocking out the first exon at N-terminal in a plant can improve the saline-alkali tolerance of the plant.

In some embodiments, the N-terminal GGL domain or GGL-like domain of AT1 or homologous gene thereof is conserved.

In some embodiments, the present invention provides a nucleic acid molecule, which encodes a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15, and which is used to regulate the saline-alkali tolerance of a plant, or to produce a plant with saline-alkali tolerance or salt-alkali sensitivity.

In some embodiments, the nucleic acid molecule encodes a protein comprising an amino acid sequence as set forth in any one of SEQ ID NOs: 15 and 86-96.

In some embodiments, the nucleic acid molecule encodes an amino acid sequence selected from:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85.

The present invention also provides a mutant protein, which is encoded by a variant nucleic acid molecule generated from a frameshift mutation occurring in a nucleic acid molecule encoding the following amino acid sequence:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85;
wherein the mutant protein has a reduced activity or no activity as compared with the protein encoded by the nucleic acid molecule before the frameshift mutation.

In some embodiments, the frameshift mutation comprises an insertion or deletion of one or more (not 3 or multiples of 3) nucleotides. The frameshift mutation may occur at any position in the target coding sequence.

The present invention also provides a nucleic acid molecule encoding the mutant protein.

In some embodiments, the present invention provides an expression cassette, which comprises the nucleic acid molecule of the present invention.

In some embodiments, the present invention provides a recombinant vector, which comprises the nucleic acid molecule or expression cassette of the present invention.

In some embodiments, the present invention provides a cell, which comprises the nucleic acid molecule, expression cassette or recombinant vector of the present invention.

In some embodiments, the cell is selected from a prokaryotic cell or a eukaryotic cell, and the prokaryotic cell is, for example, a bacterial cell or a fungal cell, for example, but not limited to, an Escherichia coli cell, a yeast cell or an Agrobacterium cell; the eukaryotic cell is, for example, a plant cell.

In a second aspect, the present invention provides a method for producing a saline-alkali-tolerant plant, the method comprising:
reducing the expression level of all alleles encoding a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15, or making the all alleles non-expressed in the plant,
wherein the growth of the saline-alkali-tolerant plant under salt-alkali conditions is better than that of a wild-type plant,
wherein the salt-alkali conditions comprise growth conditions at pH >7.5 and Na⁺ concentration >75 mM or growth conditions at pH >8.0 and Na⁺ concentration >50 mM.

In some embodiments, reducing the expression level of the all alleles or making the all alleles non-expressed in the plant is performed by gene editing method, targeted mutagenesis, chemical induction, radiation induction, natural mutation, RNAi or addition of a substance capable of inhibiting the expression of the target gene.

In some embodiments, the all alleles in the plant are knocked out or mutated, for example, a GGL domain or GGL-like domain of the gene is knocked out by homologous recombination, or a GGL domain or GGL-like domain of the gene is edited by CRISPR technology.

In some embodiments, the first exon portion at N-terminal of the all alleles is knocked out or mutated, so that the encoded protein has a reduced or no activity.

In some embodiments, the GGL domain or GGL-like domain, or the first exon portion at N-terminal of the all alleles is knocked out or mutated, so that the encoded protein has a reduced or no activity.

In some embodiments, the gene has an expression level reduced by at least 51%, preferably 60%, 70% or 80%, more preferably 85%, 90% or 95%, or even is not expressed, as compared to a wild-type control plant.

In some embodiments, the plant is a monocotyledonous plant or a dicotyledonous plant, for example, a Gramineae plant, for example, but not limited to, a sorghum genus plant (e.g., sorghum), an Oryza plant (e.g., rice), a millet, a maize, a wheat or a soybean.

In some embodiments, the method further comprises identifying an offspring plant of one or more generations obtained by selfing a parent plant comprising a knockout or mutation of the all alleles or comprising non-functional allele(s), or crossing it with another parent plant comprising a knockout or mutation of the all alleles or comprising non-functional allele(s), wherein the knockout or mutation of the all alleles results in that the protein encoded by the all alleles has a reduced or no activity.

In some embodiments, the GGL domain or GGL-like domain, or the first exon portion at N-terminal of all alleles of a gene encoding an amino acid sequence selected from the following amino acid sequences is knocked out or mutated:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85.

In some embodiments, the saline-alkali-tolerant plant produced by the method of the present invention has an improved survival rate, an improved yield, an improved plant height or a fresh weight under saline-alkali growth conditions as compared with the corresponding wild-type control.

In some embodiments, the saline-alkali growth conditions comprise culture conditions at pH>7 and Na⁺ concentration >50 mM.

In a third aspect, the present invention provides a plant or plant material, wherein all alleles encoding a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15 are knocked out or mutated, and preferably wherein a GGL domain or GGL-like domain or the first exon portion at the N-terminal of the all alleles of the gene is knocked out or mutated,
wherein the knockout or mutation in the all alleles results in that the protein encoded by the all alleles has a reduced or no activity.

In some embodiments, the plant or plant material comprises a non-functional allele of the gene.

In some embodiments, the plant is a monocotyledonous plant or a dicotyledonous plant, for example, a Gramineae plant, such as, but not limited to, a Sorghum genus plant (e.g., sorghum), Oryza plant (e.g., rice), millet, maize, wheat, or soybean.

In some embodiments, the plant material is a plant part, plant organ, plant tissue, seed, plant protoplast, or plant cell, for example, an embryo, pollen, ovule, seed, leaf, flower, branch, fruit, stem, root, root tip, anther, plant cell culture, or plant callus.

In some embodiments, in the plant or plant material, the GGL domain or GGL-like domain, or the first exon portion at N-terminal of the all alleles of the gene encoding an amino acid sequence selected from the following amino acid sequences is knocked out or mutated:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85.

In a fourth aspect, the present invention provides a method for producing seeds of a hybrid plant, the method comprising:
(i) hybridizing a first parent plant with a second parent plant, wherein in the first parent plant and the second parent plant, all alleles encoding a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15 are knocked out or mutated; and
(ii) harvesting seeds of the hybrid plant or progeny thereof.

In some embodiments, the first parent plant and/or the second parent plant comprises a non-functional allele of the gene.

In some embodiments, the first parent plant and/or the second parent plant is an inbred line plant.

The present invention also provides a method for producing seeds of a conventional breeding plant, the method comprising:
propagating a parent seed to harvest offspring seeds thereof, wherein in the parent seeds, all alleles encoding a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15 have been knocked out or mutated, so that the protein is not expressed or has a reduced expression level as compared with that of a wild-type plant.

In some embodiments, in the parental seed, all alleles encoding a protein comprising one of the following amino acid sequences have been knocked out or mutated:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85.

In the fifth aspect, the present invention provides a plant grown from the seed of the fourth aspect, or plant material thereof.

In a sixth aspect, the present invention provides a method for producing a salt-alkali sensitive plant, the method comprising:
increasing the expression level of a gene encoding a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, more preferably at least about 90% or about 95% similarity to SEQ ID NO: 15 in a plant, or expressing or overexpressing a C-terminal truncated protein encoded by the gene mutant in the plant.

In some embodiments, the expression level of the target gene is increased by introducing into the plant an exogenous nucleic acid molecule encoding a protein comprising an amino acid sequence having at least 40% identity, preferably at least about 75% identity, more preferably at least about 80% identity, or more preferably at least about 90% or about 95% identity, or having at least about 60% similarity, preferably at least about 70%, about 80% or about 85% similarity, and more preferably at least about 90% or about 95% similarity to SEQ ID NO: 15.

In some embodiments, a genetic material carrying the nucleic acid molecule is introduced into the cell or tissue of the plant, and the genetic material exists in the plant in the form of free or integrated into the chromosome of the plant, and the cell or tissue with the genetic material introduced is cultured into a whole plant to obtain the salt-alkali sensitive plant.

In the present invention, the saline-alkali conditions comprise growth conditions at pH >7.5 and Na⁺ concentration >75 mM or growth conditions at pH >8.0 and Na⁺ concentration >50 mM.

The embodiments of the present invention will be described in details below in conjunction with the accompanying drawings and examples, but those skilled in the art will understand that the following drawings and examples are only used to illustrate the present invention, rather than to limit the scope of the present invention. According to the following detailed description of the drawings and preferred embodiments, various objects and advantages of the present invention will become apparent to those skilled in the art.

### BRIEF DESCRIPTION OF THE DRAWINGS

FIG. 1 shows the evaluation conditions and phenotypic variation for identification of saline-alkali tolerance of populations of different sorghum lines; wherein (A) shows the survival rate analysis of the seeds of 16 sorghum lines under different concentrations of saline-alkali stress at the germination stage; and (B) to (F) show the treatment of the seeds of different sorghum lines with 75 mM mixed alkali solution (i.e., NaHCO₃:Na₂CO₃ at a molar ratio of 5:1, pH=9.2 to 9.4), wherein (B) shows the relative survival rate (RSR) analysis of 16 sorghum lines under alkaline stress for 5 consecutive weeks; (C) shows the phenotypic observation of representative sorghum lines treated with alkaline stress for 3 weeks or without alkaline stress (i.e., CK, control), in which the scale bar in the figure represents 5 cm; (D) shows the number of sorghum selfing line materials (sorghum accessions) from the sorghum association panel (SAP) with different relative survival rates on the 21^{st} day after treatment with 75 mM mixed alkali solution; (E) shows the relative survival rates of 352 grain sorghum natural population materials under control (CK) and alkali treatment conditions (treatment with 75 mM mixed alkali solution), in which the lines with low germination rates (<80%) in the control were removed for counting; and (F) shows the QQ-plot analysis of saline-alkali tolerance in SAP population.

FIG. 2 shows the phenotypic changes of sorghum under different salinity and alkalinity levels; wherein (A) shows the statistical analysis of seedling rates of 16 test materials under different concentrations of mixed alkaline stress; (B) shows the relative survival rate analysis of 16 test materials under 75 mM mixed alkaline stress and control conditions for different days, in which CK represents the survival rate under control conditions, T represents the survival rate under 75 mM mixed alkaline stress, and T_CK represents the relative survival rate.

FIG. 3 shows the natural variation of SbAT1 gene associated with saline-alkali tolerance in sorghum, wherein (A) shows the Manhattan plot of a genome wide association study (GWAS) for alkali tolerance in the natural sorghum association panel population, in which the relative survival rates were obtained from sorghum plants sown and grown for 21 days with (alkaline stress) and without (control) the addition of 75 mM mixed alkali solution, in which the arrow indicates the major locus of SbAT1 gene; (B) shows the scatter plot of the SbAT1 locus flanked by an ˜10 Mb genome region on Chromosome 1; (C) shows SbAT1-based association mapping between the 29 detected sequence variations in the SbAT1 gene region and the alkali tolerance of 37 sequenced sorghum accessions, in which the LD analysis between the 29 causal sites indicates linkage association signals; and the five leading variant sites (red dots) show the strong association signals with strong LD and are highlighted with black lines; (D) two typical haplotypes (Hap1 and Hap2) of SbAT1 that were detected based on the five leading variant sites, in which the frameshift mutation in Hap2 (from “G” to “GGTGGC”) is highlighted in red; (E) shows the relative survival rates of 20 sorghum accessions in Hap1 and 17 sorghum accessions in Hap2 subjected to alkaline stress (treatment with 75 mM mixed alkali), in which n indicates number, and statistical P values are determined by two-tailed unpaired t-test; and (F) shows the relative expression levels of SbAT1 in alkaline-tolerant (T) and alkaline-sensitive(S) sorghum lines after 5 and 8 days of stress in the 75 mM mixed alkali treatment and in the corresponding control condition (CK), in which the statistical significance is determined by one-way ANOVA with Tukey's multiple comparison test, and NS indicates not significant.

FIG. 4 shows the schematic presentation of the protein structure of sorghum AT1 and its mutant at 1.

FIG. 5 shows the phenotypes of sorghum NIL parental lines and SbAT1 transgenic plants under alkali and/or salt stress treatment, wherein (A) shows the phenotypes of NIL parental lines SN010 (NIL-AT1) and M-81E (NIL-at1) under alkaline stress, in which the seeds of SN010 (NIL-AT1) and M-81E (NIL-at1) were sown in soil without or with 75 mM mixed alkali and photographed 17 days later, and the scale bar represents 5 cm; (B) and (C) show the statistical analysis of relative survival rates (B) and relative plant heights (C) of SN010 (NIL-AT1) and M-81E (NIL-at1) under alkali treatment as compared with those without alkali treatment (CK); (D) shows the phenotypes of sorghum NIL parental lines SN010 (NIL-AT1) and M-81E (NIL-at1) under saline stress, in which the seeds of SN010 (NIL-AT1) and M-81E (NIL-at1) were sown in neutral pH soil containing 75, 100, 150, and 200 mM NaCl, and photographed after 14 days, and the scale bar represents 5 cm; (E) and (F) show the statistical analysis of relative survival rates (B) and relative plant heights (C) of SN010 (NIL-AT1) and M-81E (NIL-at1) under the salt treatment of (D); (G) shows the qRT-PCR assay of SbAT1 expression in TO generation of SbAT1 overexpression (SbAT1-OE) line; (H) shows the phenotypic analysis of SbWT, SbAT1-OE, and SbAT1^{ko} under control (CK) and 75 mM mixed alkaline stress, in which photographs were taken at 14 days after sowing, and the scale bar represents 5 cm; and (K) shows the Western blot analysis of SbAT1 and Sbat1, in which the upper panel shows the schematic presentation of SbAT1 and its truncated form Sbat1 fused with GFP at the C-terminal, and the large subunit of ribulose-1,5-bisphosphate carboxylase/oxygenase (RbcL) was used as the loading control.

FIG. 6 shows the function of SbAT1 in alkaline tolerance in sorghum, wherein (A) shows the protein schematic presentation of SbAT1 and its truncated form Sbat1 in sorghum NIL-AT1 and NIL-at1 plants; (B) shows the phenotypic analysis of sorghum NIL seedlings under alkaline stress, in which photographs were taken 14 days after seed sowing (CK, no alkaline stress; 75 mM mixed alkali), and the scale bar represents 5 cm; (C) shows the statistical analysis of relative survival rates of seedlings in (B); (D) shows the schematic representation of SbAT1 and its non-functional version in SbWT, SbAT1 overexpression (SbAT1-OE), and SbAT1 knockout (SbAT1^{ko}) plants; (E) shows the photographs of representative seedlings of SbWT, SbAT1 overexpression (SbAT1-OE), and SbAT1 knockout (SbAT1^{ko}) plants on the 14^{th} day under no alkaline stress (CK) and 75 mM mixed alkaline stress, in which the scale bar represents 5 cm; and (F) shows the statistical analysis of relative survival rates of seedlings in (E).

FIG. 7 shows the field performance of the accessions of sorghum near-isogenic lines (NILs) sown in saline-alkali land in Ningxia, northwest China.

FIG. 8 shows the soil physical and chemical properties and the statistical data of corresponding seedling rates of the accessions of near-isogenic lines on saline-alkali land in different regions of Ningxia, China, wherein (A) and (B) show the soil physical and chemical properties of saline-alkali land in Huiwei Village and Dongfeng Village, Ningxia, China, respectively, and (C) and (D) show the statistical analysis data of the survival rates of the accessions of near-isogenic lines on saline-alkali land in Huiwei Village and Dongfeng Village, Ningxia, China, respectively.

FIG. 9 shows the construction and gene editing identification results of sorghum overexpression transgenes and mutants.

FIG. 10 shows the construction of millet mutants and the identification results of gene editing accessions.

FIG. 11 shows the concentration screening for phenotypic identification of transgenic sorghum and millet, wherein (A) shows the growth of transgenic sorghum and millet genetic accessions on the 14^{th} day after being subjected to control (CK) and different mixed alkaline stress treatments, (B) shows the statistical analysis of the survival rates of transgenic sorghum and millet genetic accessions under different mixed alkaline stress conditions, in which the upper panel shows the statistical analysis of the survival rate of transgenic sorghum, and the lower panel shows the statistical analysis of the survival rate of transgenic millet.

FIG. 12 shows the conserved function of AT1 homologues Gγ-like subunit in alkaline tolerance of millet, rice and maize, in which (A) shows the schematic presentation of millet SiAT1 and its truncated or non-functional versions in SiAT1 genetic plants, in which SiWT indicates the wild-type millet Ci846; (B) shows the photographs of representative SiAT1 genetic plants on the 14^{th} day after sowing in the absence or presence of 75 mM mixed alkaline stress, in which the scale bar represents 5 cm; (C) shows the statistical analysis of the relative survival rates of millet in (B); (D) shows the schematic presentation of rice OsGS3 and its truncated or non-functional versions in OsGS3 genetic plants, in which OsWT represents the wild-type rice ZH11; (E) shows the photographs of representative OsGS3 genetic plants on the 21^{st} day after sowing in the absence or presence of 75 mM mixed alkaline stress, in which the scale bar represents 5 cm; (F) shows the statistical analysis of the relative survival rates of rice in (E); (G) shows the schematic presentation of wild-type ZmGS3 in wild-type maize ZmWT and its non-functional versions in ZmGS3^{ko} maize, in which ZmWT is the wild-type maize KN5585; (H) shows the photographs of ZmWT and ZmGS3^{ko} maizes on the 14^{th} day after sowing in the absence or presence of 75 mM mixed alkaline stress, in which the scale bar represents 5 cm; and (I) shows the statistical analysis of the relative survival rates of maize plants in (H) on the 50^{th} day after sowing.

FIG. 13 shows the gene-editing information of AT1 in millet and maize and phenotypes of transgenic plants in response to alkaline stress, wherein (A) shows the Western blot analysis of Myc-SiAT1¹²⁴ expression in TO generation of different SiAT1¹²⁴ overexpression (SiAT1¹²⁴-OE) lines; (B) shows the target sequence location and gene-editing information of AT1 in millet SiAT1¹⁰² plants, in which one base insertion occurred in SiAT1¹⁰², resulting in a frameshift mutation and premature translation termination, and a C-terminal truncated protein with predicted amino acids 1-102 was retained in SiAT1¹⁰²; (C) shows the phenotypic analysis and statistical analysis of relative plant heights of SiWT, SiAT1¹²⁴-OE, SiAT1¹⁰² and SiAT1^{KO} millet plants under alkaline stress, in which the seeds of millet were sown in soil without or with 75 mM mixed alkali and photographed on the 14^{th} day after sowing, and the scale bar represents 5 cm; (D) shows the growth morphology of rice OsWT, OsGS3-10E, OsGS3-40E, OsGS3^{ko} and OsGS3Ri without or with 75 mM mixed alkali stress treatment, in which the photographs were taken on the 29^{th} day after sowing, and the scale bar represents 5 cm; (E) and (F) show the statistical analysis of the relative plant height (E) and relative chlorophyll content (F) of rice lines in (D), in which the data were the mean±SEM of 4 representative plants of each line, and the statistical significance was determined by one-way ANOVA with Tukey's multiple comparison test; (G) shows the target sequence location and gene-editing information of ZmGS3 and ZmGS3^{ko} maize plants, in which 34 bp deletion and one base mutation occurred in ZmGS3^{ko} maize plants, resulting in frameshift mutation and premature translation termination; (H) shows the phenotypic analysis and relative plant height statistical analysis of maize ZmWT and ZmGS3^{ko} plants under alkaline stress treatment, in which the seeds of maize were sown in soil without (CK) or with 75 mM mixed alkali and photographed on the 14^{th} day after sowing, the scale bar represents 5 cm, in which the right panel shows the statistical analysis of plant height of maize ZmWT and ZmGS3^{ko} plants under 75 mM mixed alkaline stress, and the statistical differences were determined by two-tailed unpaired t-test; and (I) the phenotypes of maize ZmWT and ZmGS3^{ko} plants under 75 mM mixed alkaline stress treatment, in which the photographs were taken on the 50^{th} day after sowing, and the scale bar represents 5 cm.

FIG. 14 shows the construction results of transgenic plants with overexpression of rice GS3-1 or GS3-4 and suppressed expression of GS3, wherein (A) shows the schematic presentation of overexpression vector; (B) shows the schematic presentation of suppressed expression vector; (C) shows the detection results of the expression levels in the transgenic plants with the overexpression and suppressed expression.

FIG. 15 shows the schematic presentation of the construction process of rice GS3 CRISPR knockout vector pYL-Cas9-gRNA-OsGS3, wherein (A) shows the schematic presentation of the structure of rice GS3 gene and the location of CRISPR target; (B) shows the process in which two targets T1 and T2 were inserted into pYL-OsU3-gRNA and pYL-OsU6a-gRNA by PCR to obtain pYL-OsU3-T1-gRNA and pYL-OsU6a-T2-gRNA; and (C) shows the structural schematic presentation of the expression vector pYL-Cas9-gRNA-OsGS3.

FIG. 16 shows the detection results of mutation sites in the T1 generation of rice GS3 CRISPR knockout transgenic plants.

FIG. 17 shows the schematic presentation of the construction process of the maize ZmGS3 CRISPR knockout vector pYL-Cas9-gRNA-ZmGS3; wherein (A) shows the schematic presentation of the structure of the maize ZmGS3 gene and the CRISPR target; (B) shows the detection results of mutation sites in the T1 generation of maize ZmGS3 CRISPR knockout transgenic plants.

FIG. 18 shows the schematic presentation of the TaGS gene structure and the target setting using CRISPR/Cas9 technology according to Example 4.1 of the present invention.

FIG. 19 shows the schematic presentation of the sequencing results of the T2 generation mutant of TaGS gene of the transgenic wheat plant E5 according to Example 4.2 of the present invention; wherein WT represents the wild-type gene sequence, “-” represents the sequence with a deletion mutation, and the number following “-” represents the number of deleted or inserted nucleotides.

FIG. 20 shows the saline-alkali tolerance phenotypes of wheat under salt-alkaline stress according to Example 4.3 of the present invention, wherein (A) shows the results of treatment with clean water; (B) shows the results of treatment with 75 mM mixed alkaline salt solution (NaHCO₃:Na₂CO₃ at a molar ratio of 5:1); the three plants on the left in (A) and (B) are wild-type Fielder wheat (i.e., target wheat), and marked as WT; and the three plants on the right in (A) and (B) are wheat TaGS gene triple-mutation mutant E5, and marked as E5.

FIG. 21 shows the gene-editing information of TaAT1 (also known as TaGS) in wheat and the phenotypes of transgenic wheat plants in response to alkaline stress, wherein (A) shows the schematic presentation of the T-DNA structure in the CRISPR/Cas9 construct; (B) shows the CRISPR/Cas9-induced target gene mutagenesis, in which the target sequence and PAM sequence are shown in blue and red, respectively, and the mutation site is shown in a dotted line; (C) shows the phenotypic analysis of wild-type wheat TaWT and TaAT1 knockout (TaAT1^{ko}) plants under alkaline stress, in which the seeds of wheat were sown in soil without or with 125 mM mixed alkali, and photographed on the 21^{st} day after sowing, and the scale bar represents 5 cm; (D) shows the statistical analysis of relative survival rates of wheat lines in (C); (E) shows the representative seedlings of TaAT1 genetic plants without or with 125 mM mixed alkaline stress treatment on the 21^{st} day after sowing; (F) shows the statistical analysis of relative plant heights of wheat lines in (E), in which the data are mean±SEM of three representative plants of each line; (G) shows the DAB staining results of leaves of wild-type wheat TaWT and TaAT1 knockout (TaAT1^{ko}) plants, in which the scale bar represents 1 cm, and the 10-day-old seedlings were treated with or without 250 mM mixed alkali for 60 h for analysis; (H) shows the H₂O₂ amounts detected by ROS detection probe (H₂DCFDA) in root tips of TaAT1-related genetic plants, in which the 10-day-old seedlings were treated with or without 250 mM mixed alkali for 48 h for analysis, and the scale bar represents 100 μm; and (I) shows the statistical analysis of H₂O₂ concentrations measured in (H), in which the data are mean±SEM (n=6 plants).

FIG. 22 shows the contribution of OsGS3 non-functional allele to alkaline tolerance in rice, wherein (A) shows the phenotypes of KY^{NIL} (GS3) and KY^{NIL} (gs3−) seedlings treated with or without 75 mM mixed alkali on the 21^{st} day after sowing, in which the scale bar represents 5 cm, and the right panel shows the statistical analysis of relative plant heights of rice under alkali treatment as compared with no alkali treatment; (B) shows the phenotypes of KY^{NIL} (GS3) and KY^{NIL} (gs3⁻) rice grown in soil, in which the scale bar represents 5 cm, the right panel shows the statistical analysis of relative survival rates, and the data are mean±SEM of four replicates per treatment, with 40 plants tested in each replicate; (C) shows the relative survival rates and grain yields of rice NIL grown in natural alkaline soil (pH 9.45) and near-neutral soil (pH 7.74) in a greenhouse in Jilin Province, China, in which the number of panicles indicates the number of panicles per plant, and the data are mean±SEM (n=3 plots); (D) shows the seedling phenotypes of KY^{NIL} (GS3) and KY^{NIL} (gs3⁻) grown in alkaline soil (pH 9.17) in Jilin Province, China in 2021; (E) shows the representative panicles of KY^{NIL} (GS3) and KY^{NIL} (gs3⁻) grown in alkaline soil (pH 9.17) in Jilin Province, China in 2021, in which the scale bar represents 2 cm; (F) shows the number of grains per panicle of KY^{NIL} (GS3) and KY^{NIL} (gs3⁻) grown in fields (pH 5.58) in Heilongjiang Province, China in 2021, in which the data are mean±SEM (n=28); (G) and (H) show the grain yields of KY^{NIL} (GS3) and KY^{NIL} (gs3⁻) grown in alkaline soil (pH 9.10) in Jilin Province and fields (pH 5.58) in Heilongjiang Province, China in 2022, in which the data are mean±SEM (n=5 plots); (I) and (J) show the grain length (I) and grain width (J) of rice KY^{NIL} (GS3) and KY^{NIL} (gs3⁻) grown in alkaline-free fields (pH 7.20) in Beijing and alkaline soil (pH 9.10) in Jilin Province, China in 2022, in which the data are mean±SEM (n=100); (K) shows the grain yield of Zhongkefa5 (ZKF5), an improved elite rice variety with OsGS3 non-functional allele, in which the rice was grown in high-sodium soil (pH 8.5-8.7) and low-sodium soil (pH 7.4-7.6) in Jilin Province, China in 2021; and (L) shows the statistical analysis of relative survival rates of OsWT and OsGS3^{ko} grown in natural alkaline soil (pH 9.45) and near-neutral soil (pH 7.74) in a greenhouse in Jilin Province, China, in which the data are mean±SEM (n=3 plots).

FIG. 23 shows that AT1 GS3 knockout and natural non-functional alleles enhance crop yield in saline-alkaline fields, wherein (A) shows the phenotypes and grain yields of rice KY^{NIL} (GS3) and KY^{NIL} (gs3⁻) grown in alkaline soil (pH 9.17) in Jilin Province, China in 2021, in which the first panel shows the phenotypes of rice plants at the reproductive stage (3 months after planting in the field), the number of panicles indicates the number of rice panicles per rice plant, and the data are mean±SEM (n=3 plots); (B) shows the phenotypes, survival rates, grain yields, and total biomasses of sorghum SbWT and SbAT1^{ko} grown in alkaline soil (pH 9.10) in Ningxia Autonomous Region, China in 2021, in which the data are mean±SEM (n=3 plots); (C) shows the phenotypes and fresh weights of sorghum NIL-SbAT1 and NIL-Sbat1 seedlings planted in alkaline soil (pH 9.10) in Ningxia, China in 2021; (D) shows the phenotypes, survival rates, and grain yields of millet lines planted in alkaline soil (pH 9.10) in Ningxia, China in 2021, in which the data are mean±SEM (n=3 plots); and (E) shows the statistical analysis of the survival rates of maize ZmWT and ZmGS^{ko} plants planted in alkaline soil (pH 9.10) in Ningxia, China in 2021, in which the data are mean±SEM (n=3 plots), and in (A) to (E), the statistical significance was determined by two-tailed unpaired 1-test, *P<0.05 and ** P<0.01, *** P<0.001 and **** P<0.0001.

FIG. 24 shows a putative model of Gγ subunit AT1-mediated response to alkaline stress in plants, in which under alkaline stress, PIP2s functions as a H₂O₂ exporter, and the Gγ subunit AT1 may pair with GB to negatively regulate the phosphorylation of PIP2s, thereby reducing the H₂O₂ export capacity of PIP2s, resulting in excessive accumulation of H₂O₂ and causing plant sensitivity to alkaline stress; the truncated form of AT1, i.e., at1, further inhibits the H₂O₂ export activity and causes high sensitivity of plants to alkaline stress; however, the natural non-functional form of AT1 or the knockout of AT1 homologous genes releases the inhibitory effect on PIP2s and effectively improves the tolerance against alkaline stress in crops.

FIG. 25 shows the amino acid sequence alignment of AT1 homologous genes of sorghum (Sb), rice (Os), millet (Si), cultivated soybean (Gm), and wild soybean (Gs), in which the part outlined by the red box is the predicted conserved GGL domain.

### DESCRIPTION OF SEQUENCE LISTING

TABLE A
Brief description of sequences in the present invention

| SEQ ID NO: | Description |
|---|---|
| 1 | Genomic sequence of sorghum AbAT1 gene |
| 2 | cDNA sequence of sorghum AbAT1 gene |
| 3 | Coding sequence (CDS) of sorghum AbAT1 gene |
| 4 | Protein sequence of sorghum AbAT1 |
| 5 | Genomic sequence of millet SiAT1 gene |
| 6 | cDNA gene of millet SiAT1 gene |
| 7 | Coding sequence (CDS) of millet SiAT1 gene |
| 8 | Protein sequence of millet SiAT1 |
| 9 | cDNA sequence of sorghum at1 gene |
| 10 | Protein sequence of sorghum at1 |
| 11 | Coding sequence expressed by sorghum AT1^{KO} plant |
| 12 | Protein sequence expressed by sorghum AT1^{KO} plant |
| 13 | Coding sequence of AT1 expressed by SiAT1^{KO} plant |
| 14 | Protein sequence expressed by SiAT1^{KO} plant |
| 15 | N-terminal GGL conserved domain of sorghum AT1 |
| 16 | Complete (GS3-1) protein sequence of rice |
| 17 | Protein sequence of rice GS3 C-terminal truncated protein (GS3-4) |
| 18 | Maize GS3 protein sequence |
| 19 | Rice GS3 genomic sequence |
| 20 | Rice GS3-1 cDNA sequence |
| 21 | Rice GS3-4 cDNA sequence |
| 22 | Maize GS3 genomic sequence |
| 23 | Maize GS3 cDNA sequence |
| 24-25 | Rice target sequences |
| 26 | Maize target sequence |
| 27-28 | Rice gRNA sequences |
| 29 | Maize gRNA sequence |
| 30 | Rice mutant gene sequence |
| 31 | Maize mutant gene sequence |
| 32 | Rice mutant protein sequence |
| 33 | Maize mutant protein sequence |
| 34 | Nucleotide sequence of GS3 gene coding sequence when forward inserted |
| 35 | Nucleotide sequence of GS3 gene coding sequence when reverse inserted |
| 36-54 | Primer sequences |
| 55 | Wheat TaGS-4A1 protein sequence |
| 56 | Wheat TaGS-4A2 protein sequence |
| 57 | Wheat TaGS-7A protein sequence |
| 58 | Wheat TaGS-7D protein sequence |
| 59 | Wheat TaGS-4A genomic sequence |
| 60 | Wheat TaGS-7A genomic sequence |
| 61 | Wheat TaGS-7D genomic sequence |
| 62 | Wheat TaGS-4A1 cDNA sequence |
| 63 | Wheat TaGS-4A2 cDNA sequence |
| 64 | Wheat TaGS-7A cDNA sequence |
| 65 | Wheat TaGS-7D cDNA sequence |
| 66 | Wheat TaGS-4A1 coding sequence (CDS) |
| 67 | Wheat TaGS-4A2 coding sequence (CDS) |
| 68 | Wheat TaGS-7A coding sequence (CDS) |
| 69 | Wheat TaGS-7D coding sequence (CDS) |
| 70 | TaGS target sequence |
| 71-72 | Primer sequences |
| 73 | Base sequence of TaGS-4A mutation deletion in wheat E5 plant |
| 74, 76, 78 | Coding sequences (CDS) of cultivated soybean AT1 homologous genes |
| 75, 77, 79 | Protein sequences encoded by cultivated soybean AT1 homologous |
| | genes |
| 80, 82, 84 | Coding sequences (CDS) of wild-type soybean AT1 homologous genes |
| 81, 83, 85 | Protein sequences encoded wild-type soybean AT1 homologous genes |

### DETAILED DESCRIPTION OF INVENTION

It should be understood by those skilled in the art that the present invention is not limited to the specific methodology, embodiments and reagents described herein, as these are exemplary illustrations. It should also be understood that the terms used herein are only for the purpose of describing specific embodiments and are not intended to limit the scope of the present invention, which is defined only by the appended claims.

Unless otherwise defined, all technical and scientific terms used herein have the same meaning as commonly understood by those of ordinary skill in the art to which the present invention belongs.

In addition, unless the context requires otherwise, terms in singular form shall include plural form thereof, and terms in plural form shall include singular form thereof. More specifically, as used in this description and the appended claims, the singular forms “a/an” and “the” include plural referents unless the context clearly indicates otherwise.

The following definitions and methods are provided to better define the present application and to guide those of ordinary skill in the art in the practice of the present application. Unless otherwise specified, the terms are understood according to the conventional usage of those of ordinary skill in the relevant art. All patent documents, academic papers, industry standards and other public publications cited herein are incorporated herein by reference in their entirety.

As used herein, “plant” broadly includes references to whole plants, plant organs, plant tissues, seeds and plant cells and their progeny. Plant cells include, but are not limited to, cells from seeds, suspension cultures, plantules, meristematic regions, calli, leaves, roots, seedlings, gametophytes, sporophytes, pollen and microspores. “Progeny” includes any subsequent generations of plants. “Rice” or “maize” is any rice or maize plant and includes all plant varieties that can be bred with rice or maize, including whole plants, plant cells, plant organs, plant protoplasts, plant cell tissue cultures from which plants can be regenerated, plant calli, intact plant cells in plants or plant parts, and the examples of plant parts include embryos, pollen, ovules, seeds, leaves, flowers, branches, fruits, stems, roots, root tips, anthers, etc.

As used herein, the terms “saline-alkali land/field”, “sodium-containing soil” or “alkaline soil” refer to types of soil in which salts accumulate, indicating that the salts contained in the soil affect the normal growth of plants (e.g., crops). The formation of alkaline soil and alkalized soil is mostly related to the accumulation of carbonates in the soil, so the alkalinity is generally high, and plants can hardly survive in areas with severe saline-alkali soil. According to the salt content and pH value of soil, saline-alkali land is classified as mild saline-alkali land, moderate saline-alkali land and severe saline-alkali land. Among them, mild saline-alkali land refers to a land with a germination rate of 70% to 80% when planting crops and a salt content of less than 3/1000; severe saline-alkali land refers to a land with a salt content of more than 6/1000 and a germination rate of less than 50%; moderate saline-alkali land is between mild saline-alkali land and severe saline-alkali land; when expressed in pH value: the pH value of mild saline-alkali land is 7.1 to 8.5, the pH value of moderate saline-alkali land is 8.5 to 9.5, and the pH value of severe saline-alkali land is 9.5 or above. Mixed sodium salts (e.g., sodium carbonate and sodium bicarbonate) are commonly used in the laboratory to simulate “sodium-containing soil”.

In the present application, the wordings “comprise”, “include” or their variations should be understood to include other elements, numbers or steps in addition to the elements, numbers or steps described.

Unless otherwise specified, nucleic acids are written from left to right in the direction from 5′ to 3′; amino acid sequences are written from left to right in the direction from amino to carboxyl terminal. Amino acids may be represented herein by their commonly known three-letter symbols or by the single-letter symbols recommended by the IUPAC-IUB Biochemical Nomenclature Commission. Similarly, nucleotides may be represented by the commonly accepted single-letter codes. Numerical ranges include the numbers defining the ranges.

As used herein, “nucleic acid” includes deoxyribonucleotides or ribonucleotide polymers in single-stranded or double-stranded form, and unless otherwise limited, includes known analogs (e.g., peptide nucleic acids) having the essential properties of natural nucleotides, and the analogs are capable of hybridizing with single-stranded nucleic acids in a manner similar to naturally occurring nucleotides.

As used herein, when used in the context of a particular nucleic acid, the term “encoding” or “encoded” means that the nucleic acid comprises the necessary information to direct the translation of the nucleotide sequence into a particular protein. Codons are used to represent information encoding proteins. As used herein, “full-length sequence” in reference to a particular polynucleotide or a protein encoded thereby refers to the entire nucleic acid sequence or the entire amino acid sequence having a natural (non-synthetic) endogenous sequence. The full-length polynucleotide encodes the full-length, catalytically active form of a particular protein.

The terms “polypeptide,” “peptide,” and “protein” are used interchangeably herein to refer to a polymer of amino acid residues. The terms are used for an amino acid polymer in which one or more amino acid residues are artificial chemical analogs of the corresponding naturally occurring amino acids. The terms are also used for naturally occurring amino acid polymers.

The terms “residue” or “amino acid residue” or “amino acid” are used interchangeably herein to refer to an amino acid that is incorporated into a protein, polypeptide, or peptide (collectively, “protein”). The amino acid may be a naturally occurring amino acid and, unless otherwise limited, may include known analogs of a natural amino acid, and the analogs may function in a manner similar to that of the naturally occurring amino acid.

In some embodiments, the nucleotide sequence of the present application can be changed to perform a conservative amino acid substitution. The principles and examples of the conservative amino acid substitution are further described below. In certain embodiments, the nucleotide sequence of the present application can be subjected to a substitution without changing amino acid sequence according to the disclosed monocot codon preference, for example, the codons encoding the same amino acid sequence can be substituted with the codons preferred by monocot plants without changing the amino acid sequence encoded by the nucleotide sequence. In some embodiments, a portion of the nucleotide sequence in the present application is replaced with different codons encoding the same amino acid sequence, thereby not changing the amino acid sequence encoded by the nucleotide sequence while changing the nucleotide sequence. Conservative variants include those sequences that encode an amino acid sequence of one of the proteins of the embodiments due to genetic codon degeneracy. In some embodiments, a portion of the nucleotide sequence in the present application is replaced according to monocot plant preference codons. Those skilled in the art will recognize that amino acid additions and/or substitutions are generally based on the relative similarity of amino acid side chain substituents, for example, the hydrophobicity, charge, size, etc. of the substituents. Exemplary amino acid substitution groups having various of the aforementioned contemplated properties are well known to those skilled in the art, and include arginine and lysine; glutamic acid and aspartic acid; serine and threonine; glutamine and asparagine; and valine, leucine, and isoleucine. Guidance on appropriate amino acid substitutions that do not affect the biological activity of the protein of interest can be found in the model of Dayhoff et al. (1978), Atlas of Protein Sequence and Structure (Natl. Biomed. Res. Found., Washington, D.C.), which is incorporated herein by reference. Conservative substitutions can be performed, for example, by substituting one amino acid with another amino acid with similar property. “Conservative amino acid substitutions” are those substitutions in which an amino acid is substituted with a different amino acid where the substitutions are predicted to have the least interference with the properties of the reference polypeptide. In other words, conservative amino acid substitutions substantially preserve the structure and function of the reference polypeptide. Table B below provides a list of exemplary conservative amino acid substitutions contemplated herein.

TABLE B
Exemplary conservative substitutions of amino acids

| Original residue | Conservative substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

With respect to proteins, “deletion” refers to a change of amino acid sequence that results in the absence of one or more amino acid residues. A deletion may remove at least 1, 2, 3, 4, 5, 10, 20, 50, 100, 200 or more amino acid residues. Deletions may include internal deletions and/or terminal deletions (e.g., N-terminal truncation, C-terminal truncation, or both of a reference polypeptide). A “variant,” “mutant,” or “derivative” of a reference polypeptide sequence may include a deletion relative to the reference polypeptide sequence.

With respect to proteins, a “fragment” is a portion of an amino acid sequence that is identical in sequence to a reference sequence but is shorter in length than the reference sequence. A fragment may contain up to the entire length of the reference sequence, minus at least one amino acid residue. For example, a fragment may contain 5 to 1000 consecutive amino acid residues of a reference polypeptide, respectively. In some embodiments, a fragment may contain at least 5, 10, 15, 20, 25, 30, 40, 50, 60, 70, 80, 90, 100, 150, 250, or 500 consecutive amino acid residues of a reference polypeptide. Fragments may be preferentially selected from certain regions of a molecule. The term “at least one fragment” includes a full-length polypeptide. Relative to a full-length protein, a fragment may comprise an N-terminal truncation, a C-terminal truncation, or both. A “variant”, “mutant” or “derivative” of a reference polypeptide sequence may include fragments of the reference polypeptide sequence.

With respect to proteins, the terms “insertion” and “addition” refer to a change in an amino acid sequence that result in the addition of one or more amino acid residues. An insertion or addition may refer to 1, 2, 3, 4, 5, 10, 20, 30, 40, 50, 60, 70, 80, 90, 100, 150, 200 or more amino acid residues. A “variant”, “mutant” or “derivative” of a reference polypeptide sequence may include insertions or additions relative to the reference polypeptide sequence. Variants of proteins may have N-terminal insertions, C-terminal insertions, internal insertions, or any combination of N-terminal insertion, C-terminal insertions, and internal insertions.

With respect to proteins, the terms “percent identity” and “% identity” refer to a percentage of residues that match between at least two amino acid sequences aligned using a standardized algorithm. Methods of amino acid sequence alignment are well known. Some alignment methods take into account conservative amino acid substitutions. Such conservative substitutions, explained in more detail below, generally preserve charge and hydrophobicity at the site of substitution, thereby preserving the structure (and therefore function) of the polypeptide. The percent identity of an amino acid sequence can be determined as understood in the art (see, for example, U.S. Pat. No. 7,396,664, which is incorporated herein by reference in its entirety). The Basic Local Alignment Search Tool (BLAST) of the National Center for Biotechnology Information (NCBI) provides a commonly used and freely available set of sequence comparison algorithms, which is available from multiple sources, including NCBI, Bethesda, Md., on their websites. The BLAST software suite comprises various sequence analysis programs, including “blastp,” which is used to align a known amino acid sequence with other amino acid sequences from various databases.

With respect to proteins, the percent identity can be measured over the length of the entire defined polypeptide sequence (e.g., as determined by a particular SEQ ID number), or can be measured over a shorter length, for example, a length of a fragment taken from a larger, defined polypeptide sequence (e.g., a fragment of at least 15, at least 20, at least 30, at least 40, at least 50, at least 70, or at least 150 consecutive residues). Such lengths are exemplary only, and it should be understood that any fragment length supported by the sequences shown in the tables, figures, or sequence listings herein can be used to describe the length over which the percent identity can be measured.

With respect to nucleic acids and proteins, the term “similarity” refers to a proportion of identical bases or amino acids shared by a detected sequence and a reference sequence in the entire sequence (relatively macroscopic description). In an amino acid sequence alignment, similarity also comprises, in addition to identical residues, whether two residues at corresponding position have similar properties, such as size, charge, hydrophilicity, etc. of side chain groups. In other words, for proteins, “identity” requires that the amino acids at the aligned position are exactly the same, while “similarity” does not require that the amino acids at the aligned position are exactly the same, and the amino acids at the position are considered to be similar when the amino acids at the aligned position pertain to residues for conservative substitution.

“Protein tag” refers to a polypeptide or protein that is fused together and expressed with a target protein using a DNA in vitro recombination technology to facilitate the expression, detection, tracing and/or purification of the target protein. Protein tags include but are not limited to Flag tag, His tag, MBP tag, HA tag, myc tag, GST tag and/or SUMO tag.

As used herein, “nucleic acid sequence identity” refers to a sequence similarity between two polynucleotide sequences. When a position in each of two compared sequences is occupied by the same base, for example, if a position in each of two DNA molecules is occupied by adenine, then the molecules are identical at that position. The percent identity between two nucleic acid sequences is a function of the number of matching or homologous positions shared by the two nucleic acid sequences divided by the number of positions compared×100.

The identification of nucleic acid sequence identity comprises hybridization techniques. For example, all or part of a known nucleotide sequence is used as a probe for selective hybridization with other corresponding nucleotide sequences, and the other corresponding nucleotide sequences are present in a cloned genomic DNA fragment or a cDNA fragment population (i.e., a genomic library or a cDNA library) from a selected organism. The hybridization probe can be a genomic DNA fragment, a cDNA fragment, an RNA fragment, or other oligonucleotides, and can be labeled with a detectable group such as 32P or other detectable labels. Thus, for example, a hybridization probe can be prepared by labeling a synthetic oligonucleotide based on an embodiment sequence. Methods for preparing hybridization probes and constructing cDNA and genomic libraries are generally known in the art. The hybridization of the sequences can be performed under stringent conditions. As used herein, the term “stringent conditions” or “stringent hybridization conditions” refers to the following conditions, that is, under such conditions, relative to hybridization with other sequences, the probe will hybridize to its target sequence to a detectable and greater degree (e.g., at least 2 times, 5 times, or 10 times that of the background). Stringent conditions are sequence-dependent and vary in different environments. By controlling hybridization stringency and/or controlling washing conditions, a target sequence that is 100% complementary to the probe can be identified (homologous probe method). Alternatively, stringent conditions can be adjusted to allow some sequence mismatches in order to detect lower similarities (heterologous probe method). Typically, the probe length is less than about 1000 or 500 nucleotides. Typically, stringent conditions are the following conditions, that is, under such conditions, the salt concentration is less than about 1.5 M Na ion, typically a concentration of about 0.01 M to 1.0 M Na ion (or other salts) at pH 7.0 to 8.3, and the temperature conditions are: when used for short probes (e.g., 10 to 50 nucleotides), at least about 30° C.; when used for long probes (e.g., greater than 50 nucleotides), at least about 60° C. Stringent conditions can also be achieved by adding a destabilizing agent such as formamide. Exemplary low stringency conditions comprise hybridization using 30% to 35% formamide buffer, 1 M NaCl, 1% SDS (sodium dodecyl sulfate) at 37° C., and washing with 1× to 2×SSC (20×SSC=3.0 M NaCl/0.3 M trisodium citrate) at 50° C. to 55° C. Exemplary moderate stringency conditions comprise hybridization in 40% to 45% formamide, 1.0 M NaCl, 1% SDS at 37° C., and washing in 0.5× to 1×SSC at 55° C. to 60° C. Exemplary high stringency conditions comprise hybridization in 50% formamide, 1 M NaCl, 1% SDS at 37° C., and performing final wash in 0.1×SSC at 60° C. to 65° C. for at least about 20 minutes. Optionally, wash buffers may contain about 0.1% to about 1% SDS. The duration of hybridization is generally less than about 24 hours, typically about 4 hours to about 12 hours. Specificity generally depends on the wash after hybridization, and the critical factors are the ionic strength and temperature of the final wash solution. The Tm (thermodynamic melting point) of a DNA-DNA hybrid can be approximated by the formula of Meinkoth and Wahl (1984) Anal. Biochem. 138:267-284: Tm=81.5° C.+16.6 (log M)+0.41 (% GC)−0.61 (% formamide)−500/L; wherein M is the molar concentration of monovalent cations, % GC is the percentage of guanosine and cytosine nucleotides in DNA, “% formamide” is the percentage of formamide in hybridization solution, and L is the base pair length of the hybrid. Tm is a temperature (under defined ionic strength and pH) at which 50% of the complementary target sequence hybridizes to a perfectly matched probe. Washing is generally carried out at least until equilibrium is achieved and a low background level of hybridization is achieved, for example, for 2 hours, 1 hour, or 30 minutes. Each 1% mismatch should reduce Tm by about 1° C.; thus, Tm, hybridization, and/or wash conditions can be adjusted to hybridize to sequences with the desired identity. For example, if a sequence with >90% identity is required, Tm can be reduced by 10° C. Typically, stringent conditions are selected to be about 5° C. lower than the Tm of a specific sequence and its complementary sequence at a determined ionic strength and pH. However, under very stringent conditions, hybridization and/or washing can be performed at 4° C. lower than the Tm; under moderately stringent conditions, hybridization and/or washing can be performed at 6° C. lower than the Tm; under low stringent conditions, hybridization and/or washing can be performed at 11° C. lower than the Tm.

The term “frameshift mutation” refers to a mutation that causes a series of coding sequences after an insertion or loss site to be misplaced when one or more (not 3 or multiples of 3) base pairs are inserted or lost at a certain site in a DNA fragment. It can cause abnormal genetic information after the site. When a gene with a frameshift mutation is expressed, the amino acid sequence that constitutes the polypeptide chain can be changed, thereby seriously affecting the structure and function of the protein or enzyme.

Unless otherwise specified, all numbers used in the description and claims to indicate ingredient amounts, reaction conditions, etc. should be understood as modified by the term “about” in all cases. As used herein, the term “about”, when referring to a measurable value such as mass, weight, time, volume, concentration or percentage, is meant to encompass variations of +20% from the specified amount in some embodiments, +10% from the specified amount in some embodiments, +5% from the specified amount in some embodiments, +1% from the specified amount in some embodiments, +0.5% from the specified amount in some embodiments, and +0.1% from the specified amount in some embodiments, as such variations are suitable for performing the disclosed methods and/or using the disclosed compositions, nucleic acids, polypeptides, etc. Therefore, unless otherwise indicated, the numerical parameters listed in the description and the appended claims are approximate values that may vary depending on the desired properties sought to be obtained by the subject matter disclosed in the present application.

### EXAMPLES

The embodiments of the present invention will be described in details below in conjunction with the examples, but those skilled in the art will understand that the following examples are only used to illustrate the present invention and are not intended to limit the scope of the present invention. Modifications or substitutions made to the methods, steps or conditions of the present invention without departing from the spirit and substance of the present invention are within the scope of the present application. Unless otherwise specified, the examples were based on conventional experimental conditions, for example, the conditions of Sambrook J & Russell D W, Molecular cloning: a laboratory manual, 2001, or the conditions recommended by the manufacturer's instructions. Unless otherwise specified, the chemical reagents used in the examples were conventional commercial reagents, and the technical means used in the examples were conventional means well known to those skilled in the art.

### Example 1. Discovery and Genetic Analysis of Sorghum Saline-Alkali Tolerance Gene AT1

### 1.1 Identification of Saline-Alkali Tolerance of Sorghum Natural Population SAP (Sorghum Association Panel) Accessions

352 grain sorghum accessions and 38 sweet sorghum natural population accessions were collected from the germplasm resource system of the USDA-ARS (United States Department of Agriculture-Agricultural Research Service). The freshly harvested seeds were air-dried, and after they were naturally air-dried, they were placed in a 55° C. oven for 5-7 days to break the dormancy of the seeds.

To simulate various saline-alkali conditions that might exist in saline-alkali soils in fields, we first tested a mixture of two alkali salts (NaHCO₃ and Na₂CO₃) at different concentrations (0, 25, 50, 75, 100, 125, and 150 mM) and examined their effects on sorghum seedling survival rates.

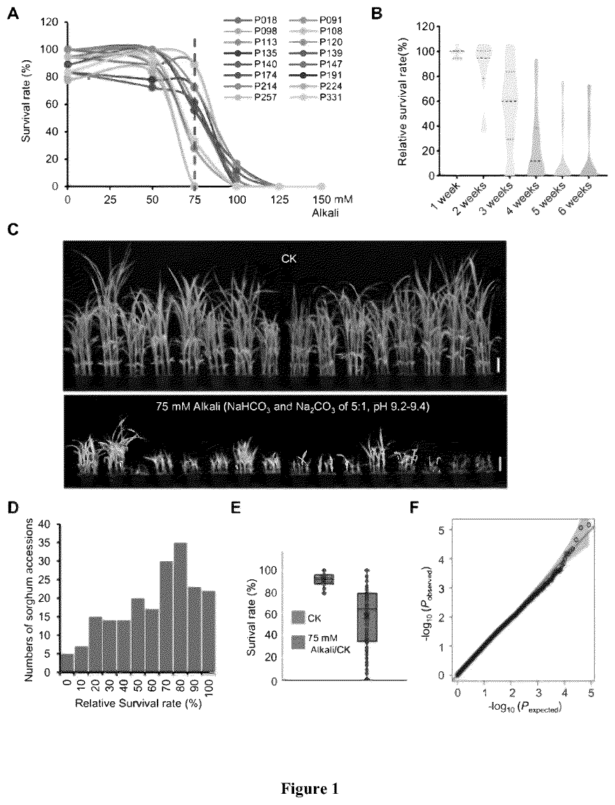

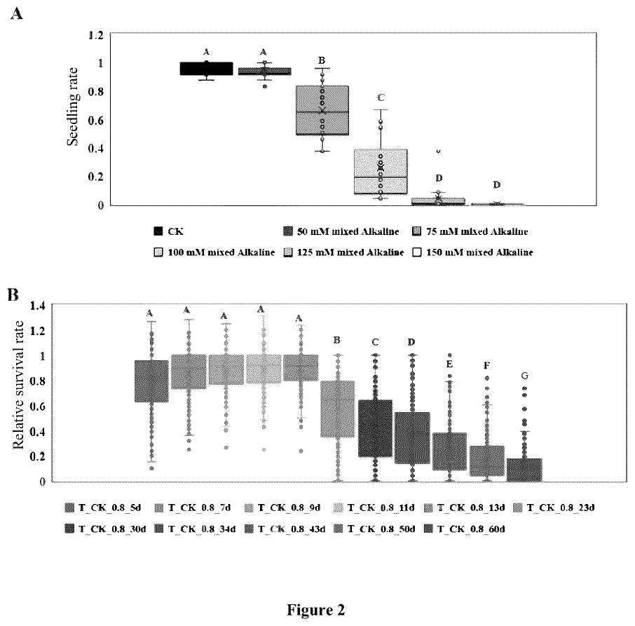

The use of the mixture of two alkali salts could produce a relatively stable pH range throughout the treatment period, which was suitable for the experiment. Different treatment periods were used in the treatments. The soil matrix was without (control) or with the mixture of two alkali salts at different concentrations (0, 25, 50, 75, 100, 125, and 150 mM), and the plant survival rates of the treated seeds were recorded on the 21^{st} day after sowing (FIG. 1A and FIG. 2). The data were the average of three replicates per treatment, and 9 plants were tested in each replicate. The relative survival rate analysis (number of survivors under alkaline stress treatment/number of survivors without alkaline stress treatment) showed that after 21 days of treatment, 75 mM mixed alkali solution (i.e., NaHCO₃:Na₂CO₃ at a molar ratio of 5:1, pH=9.2 to 9.4) exhibited the widest range of changes and was the most reliable treatment concentration for evaluating alkali tolerance in sorghum (FIG. 1A-C).

In the subsequent treatment, 75 mM mixed alkaline solution (i.e., NaHCO₃:Na₂CO₃ at a molar ratio of 5:1, pH=9.2 to 9.4) was selected. First, the seeds were sown in a soil matrix of vermiculite and nutrient soil mixed at a ratio of 1:1, with 12 seeds sown per hole and 3 replicates set. Next, the soil was watered with 75 mM mixed alkaline solution (NaHCO₃:Na₂CO₃ at a molar ratio of 5:1, pH-9.2 to 9.4) until the soil was saturated in treatment, and watered with clean water in the control (CK), then plug trays were placed on a flat ground for uniform absorption, in which three replicates were set for each treatment group. After sufficient absorption, the plug trays were placed in a model plant glass greenhouse, and watered with clean water later. The seedling growth environment was set as follows: the light/dark time was 16 h/8 h, the day and night temperature range was 28/26° C., and the relative humidity was 60% to 70%. On the 23^{rd} day of cultivation, the number of seedlings was counted, and the relative survival rates were calculated. The experimental results showed that the saline-alkali tolerances of different varieties during the germination period were significantly different (FIG. 1, B-F).

At the same time, the germination rates, germination indexes and alkali tolerance indexes of 16 grain sorghum lines under saline-alkaline stress during the germination period were dynamically monitored via a random selection method. The results showed that the alkali tolerances of the sorghum accessions showed significant differences under different concentration conditions, and with the increase of alkali concentration in treatment, the seedling rates of all tested sorghum accessions declined significantly, but in different decline extents. Through statistical analysis, it was found that under the control condition, the seedling rates of these 16 sorghum accessions were about 95%, with a standard deviation of 0.04, and a coefficient of variation of 0.04; when treated with 50 mM mixed alkali, there was no significant difference in the above three statistical data. When treated with 75 mM mixed alkali, the mean, standard deviation and coefficient of variation of the seedling rates of the 16 randomly selected sorghum accessions were 0.66, 0.20 and 0.30, respectively. When treated with >75 mM mixed alkali, the seedling rates dropped sharply and even approached 0. It could be seen that the phenotypic variation between the accessions was the largest when treated with 75 mM mixed alkali (see, 75 mM mixed Alkaline in FIG. 2) (FIG. 2A). Therefore, the 75 mM mixed alkali treatment was suitable for the identification of alkali tolerance in the germination period of natural populations. Dynamic monitoring of seedling rate was the simplest, fastest and most effective way to identify saline-alkali tolerance, efficient in time and laboring. Therefore, in subsequent experiments, relative seedling rate was selected as a representative indicator for evaluating alkali tolerance during germination period and used to evaluate the degree of saline-alkali tolerance. It was subsequently found that the sorghum accessions under alkali treatment had good seedling growth in the first two weeks, but there was a seedling burn phenomenon in the later stage of alkali treatment, and the 23^{rd} day was the “watershed” of this phenomenon. Therefore, the 23^{rd} day was selected as the time node for GWAS identification (FIG. 2B).

It was found in the statistical analysis of the relative seedling rates of 352 grain sorghum natural population accessions under control (CK) and alkali treatment conditions (75 mM mixed alkali treatment) that the relative survival rate distribution of the population accessions belonged to a normal distribution (FIG. 1D), and the survival rates were greatly affected after alkali treatment (as shown by T/CK in FIG. 1E) (FIG. 1E). QQ-plot analysis showed that the observed values were highly consistent with the expected values (FIG. 1F), indicating that the results were ideal, the phenotypic data were reliable, and further analysis could be performed. The genotype data of the population accessions were derived from the open access GBS sequencing raw data (Morris et al., 2013). The SelectVariants and VariantFiltration methods (QD <2.0, FS >200.0, ReadPosRankSum <20.0) of the GATK software were used to screen and identify the SNPs of each variety (DePristo et al., 2011). SNP sites in the heterozygous state were recorded as deletion, and SNPs with a deletion rate of more than 20% and a minimum allele frequency (MAF) of less than 5% were deleted. Finally, 82,430 SNP were detected and marked.

1.2 Discovery of SbAT1, a Gene Associated with Alkali Tolerance in Sorghum, by GWAS Analysis

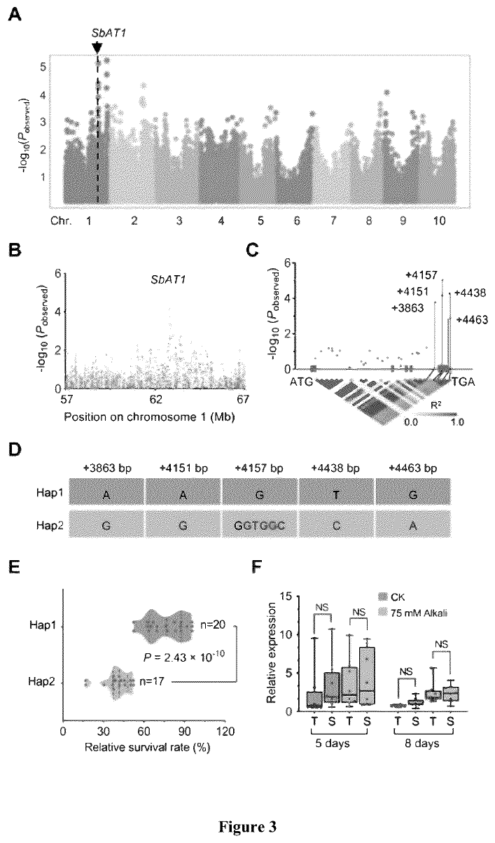

The 82,430 SNP markers detected were subjected to GWAS follow-up analysis of the population, and the results were calculated using the cMLM model (Wen et al., 2018). The results were corrected by Bonferroni and the significant correlation P value was calculated (Ranstam, 2016). Through the GWAS analysis of alkali tolerance traits of 352 grain sorghum accessions, two major loci were detected at around 55 Mb on Chromosome 1 of the grain sorghum natural population, which were extremely significantly correlated with the relative seedling rate of alkali tolerance traits (−log 10 P >5.0) (FIG. 3A and Table 1). Among them, the significant SNPs in this population: S1_5577933 and S1_55779336 were directly located inside the Sobic.001G341700 gene, which was named SbAT1 (Alkali Tolerance 1, or abbreviated as AT1) (FIG. 3B). It was preliminarily determined that SbAT1 was a major gene controlling the relative seedling rate of sorghum under saline-alkaline stress.

The genomic gene sequence of sorghum AT1 was shown in SEQ ID NO: 1. The AT1 gene contained 5 exons, of which the first exon was located at positions 1-111 of SEQ ID NO: 1; the second exon was located at positions 2447-2499 of SEQ ID NO: 1; the third exon was located at positions 2846-2890 of SEQ ID NO: 1; the fourth exon was located at positions 2972-3025 of SEQ ID NO: 1; and the fifth exon was located at positions 4084-4417 of SEQ ID NO: 1. The sequence of the cDNA gene of AT1 was shown in SEQ ID NO: 2, and its coding sequence (CDS) was shown in SEQ ID NO: 3. AT1 encoded a highly conservative protein consisting of 198 amino acids (SEQ ID NO: 4). Sequence alignment of the protein product revealed that the total length of amino acids of the gene was close to that of the homologous AT1 protein in millet (Setaria italica), and their N-terminal conservative sequences were very similar. Therefore, sorghum AT1 was considered to be the homologous gene of millet SiAT1. The genomic nucleotide sequence of millet SiAT1 gene was shown in SEQ ID NO: 5, the amino acid sequence of millet SiAT1 protein was shown in SEQ ID NO: 8, the coding cDNA sequence of the SiAT1 protein was shown in SEQ ID NO: 6, and its coding sequence (CDS) was shown in SEQ ID NO: 7.

TABLE 1
Significant SNP sites associated with alkali tolerance detected
in 352 grain sorghum natural population accessions

| Trait | SNP | Chromosome | Site (version 1.1) | P value | Gene |
|---|---|---|---|---|---|
| Survival rate | S1_55779338 | No. 1 | 55779338 | 8.71E−06 | Sobic.001G341700 |
| Survival rate | S1_55779336 | No. 1 | 55779336 | 4.69E−05 | |

### 1.3 Effect of Natural Variation of AT1 Gene on Degree of Alkali Tolerance of Sorghum

In order to verify the accuracy of the candidate gene and AT1 regulation of sorghum alkali tolerance, all variations of AT1 from 5′-UTR to 3′-UTR were retrieved, including SNP and Indel variations. Sequence alignment analysis showed that there were three variations in the 5^{th} exon, among which a 5 bp insertion appeared at the 3271^{st} nucleotide of SEQ ID NO: 1, causing premature termination of protein translation, indicating that the 5p insertion was a site that could cause functional variation of AT1 protein, and the sorghum containing this variation site was an at1 mutant (FIG. 3C). According to the 8 variations in the table in FIG. 3D, the 38 randomly selected sorghum accessions were divided into two haplotypes: the survival rate of Haplotype I (Hap1, the haplotype corresponding to the wild type of AT1 gene) sorghum under alkali treatment conditions was significantly higher than that of Haplotype II (Hap2, the haplotype corresponding to the AT1 gene in the at1 mutant) sorghum (P value=2.43×10⁻¹⁰) (FIG. 3E), and its P value of correlation with the alkali tolerance phenotype was as high as 3.31×10⁻¹⁰. Therefore, Haplotype I and Haplotype II were the alleles of alkali tolerance (AT1) and alkali sensitivity (at1), respectively (FIG. 3D). Subsequently, we randomly selected 8 alkali-tolerant and 8 alkali-sensitive lines in the grain sorghum natural population, measured the expression level of AT1 after 5 and 8 days of alkaline stress, and found that there was no significant difference in the expression of AT1 gene detected between the alkali-tolerant lines (ARL) and the alkali-sensitive lines (ASL). This showed that, consistent with the lack of strong correlation signals found in the 5′-UTR of AT1, the haplotype-based variation was indeed uncorrelated with the AT1 expression level based on RNA level (FIG. 3F). These data suggested that the alkali-tolerant and alkali-sensitive phenotypes of alkali treatment in the two AT haplotypes were not associated with the transcription level of AT1 and its variant at1, but were more likely caused by the mutations of protein numbering within the coding region.

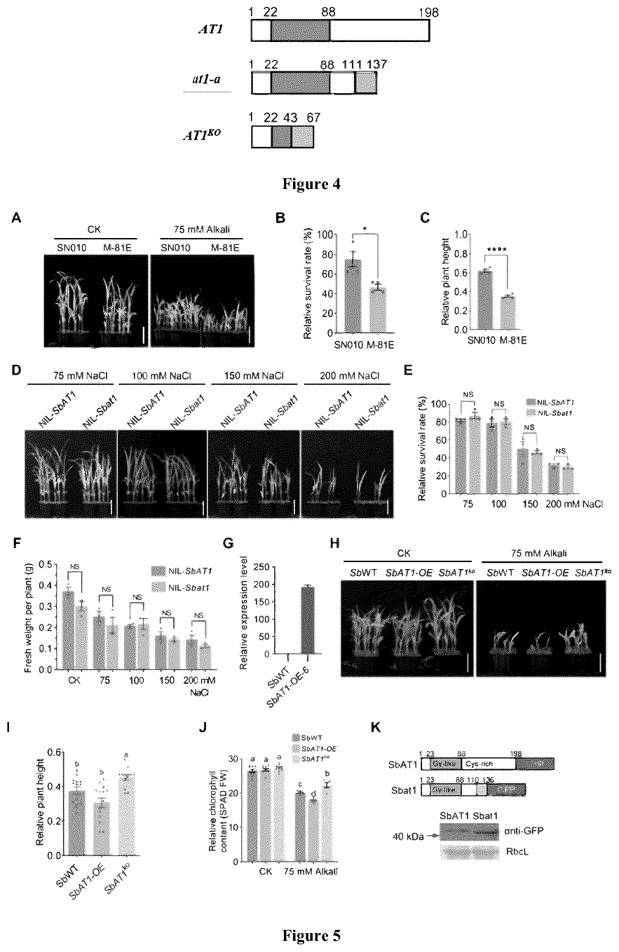

Finally, a natural mutation sequence form of the SbAT1 gene, Sbat1 gene (also referred to as the at1 gene) was detected, in which five bases (GTGGC) were inserted into the fifth exon of the wild-type AT1 gene (i.e., five nucleotides, GTGGC, were inserted between positions 3271-3272 of SEQ ID NO: 1), resulting in a frameshift mutation at the 3′ end of the AT1 gene, the premature termination of protein translation, and the formation of 137 amino acids (as shown in “at1-a” in FIG. 4). The nucleotide sequence of cDNA of the at1 gene was shown in SEQ ID NO: 9, and the amino acid sequence of the at1 protein corresponding to the AT1 gene after mutation was shown in SEQ ID NO: 10.

### 1.4 Effects of AT1 Gene on Saline-Alkali Tolerance

In order to evaluate the effects of alleles of the AT1 gene on the alkali tolerance of sorghum, we constructed and selected a pair of near-isogenic line accessions (NIL): NIL-AT1 and NIL-at1, which differed only in the genotype of the 58 kb interval on Chromosome 1, and the marker genes in the rest of the background regions were consistent. The amino acid sequence of the AT1 gene of the NIL-AT1 accession was shown in SEQ ID NO: 4; the amino acid sequence of the at1 gene of the NIL-at1 accession was shown in SEQ ID NO: 10.

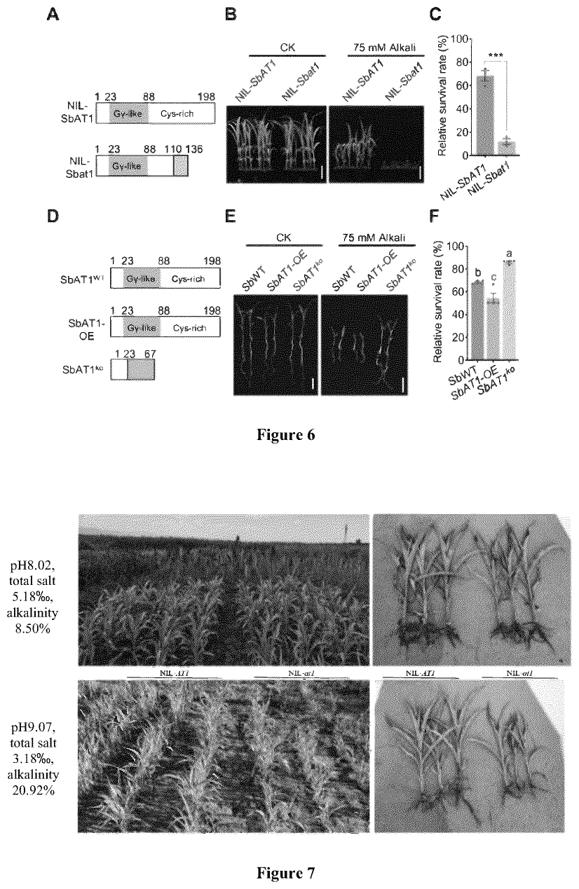

The NILs were from the cross of two sorghum germplasms, SN010 and M-81E. SN010 was haplotype Hap1 (containing wild-type AT1), M-81E was haplotype Hap2 (containing at1), and according to our survival rate and plant height data (FIG. 5A and FIG. 6A-C), SN010 (NIL-AT1) showed higher alkali tolerance than M-81E (NIL-at1). We treated the two NILs with 75 mM mixed alkali, and under alkali treatment, SN010 (NIL-AT1) showed 56.1% higher relative survival rate and better growth than M-81E (NIL-at1), but the two NILs did not show significant differences when planted in neutral pH soil (FIG. 5D-E).

As shown in FIG. 5A, there was no obvious phenotypic difference between the pair of near-isogenic line accessions under normal growth conditions without saline-alkaline stress (control, shown in CK in FIG. 5), but after 23 days of 75 mM mixed alkaline stress treatment (pH 9.32) (shown as 75 mM Alkali in FIG. 5), NIL-at1 plants were almost all dead, while NIL-AT1 had only a few leaves withered, and the plants were still alive. Then, by analyzing their phenotypes and physiological indexes, i.e., relative survival rate, plant height, fresh weight per plant, and relative chlorophyll content (SPAD) (FIG. 5B, C, J), the results showed that the alkali tolerance of NIL-AT1 was much greater than that of NIL-at1, demonstrating that AT1 was indeed involved in the response to alkaline stress.

### Single Salt NaCl Treatment of Near-Isogenic Line Accessions

It was easy for those skilled in the art to understand that the treatment with 75 mM mixed alkali not only applied alkaline stress, but also applied Na⁺ stress (i.e., salt stress) to plants. To distinguish whether the AT1-related stress phenotype of sorghum was caused by high pH alone or high sodium ion (Na⁺) concentration alone, we used 75, 100, 150 and 200 mM NaCl solutions to replace the mixed alkali (NaHCO₃ and Na₂CO₃) in neutral pH soil for similar treatments. The results showed that as the degree of salt stress increased, both NILs were sensitive to high NaCl, and the relative survival rate and single-plant fresh weight of the NIL accessions were significantly reduced. However, there was no significant difference between the two NIL accessions under different degrees of salt stress treatment (P>0.05) (FIG. 5D-F). This result indicated that the AT1lat1-related stress phenotype was more likely to be an alkali-specific response rather than a salt stress response.

To confirm this possibility, we generated additional transgenic sorghum plants in the Wheatland background (SbWT, containing the complete wild-type (WT) SbAT1 gene, kindly provided by USDA, Population genomic and genome-wide association studies of agroclimatic traits in sorghum. (2013). Proceedings of the National Academy of Science of the United States of America 110:453-458.): by overexpressing the SbAT1 gene or knocking out the SbAT1 gene via gene editing technology. The overexpression or knockout was confirmed by qRT-PCR or sequence analysis (FIG. 5G). Unexpectedly, we found that the alkali tolerance of SbAT1-overexpressing (SbAT1-OE) plants was reduced, while plants with SbAT1 knockout (SbAT1^{ko}) showed greatly improved alkali tolerance (FIG. 5H-J, FIG. 6D-F). Under 75 mM mixed alkaline stress, the survival rate of SbAT1 overexpression (SbAT1-OE) plants was 13.95% lower than that of Wheatland plants (i.e., SbWT plants), while the survival rate of SbAT1 knockout (SbAT1^{ko}) plants was 17.93% higher than that of Wheatland plants (i.e., SbWT plants) (FIG. 5F). The above phenotypes of these transgenic plants lead us to reconsider the functionality of gene mutations in the C-terminus of SbAT1 in natural variations (FIG. 3C-D), considering that the truncated mutant protein (at1) probably played a negative role in alkali tolerance. To verify our speculation, we first transiently expressed two GFP fusion proteins: SbAT1-GFP and Sbat1-GFP in plant cells to check the status of the protein accumulated with higher protein levels (FIG. 5K), thereby indicating the translation ability of the mutant at1 gene.

### Field Trial

In order to further verify the conclusion, field trials were carried out under saline-alkaline stress conditions in the natural environment. The aforementioned two sorghum NIL accessions were planted in the fields under low alkaline stress (pH 8.13, total salt content 7.68‰, alkalinity 10.71%) and high alkaline stress (pH 9.07, total salt content 3.18% 0, alkalinity 20.92%). It was found that under low alkaline stress and high alkaline stress, the NIL-AT1 accession grew better than NIL-AT1 accession, and the field trial results (FIG. 7) were consistent with the results in the greenhouse.

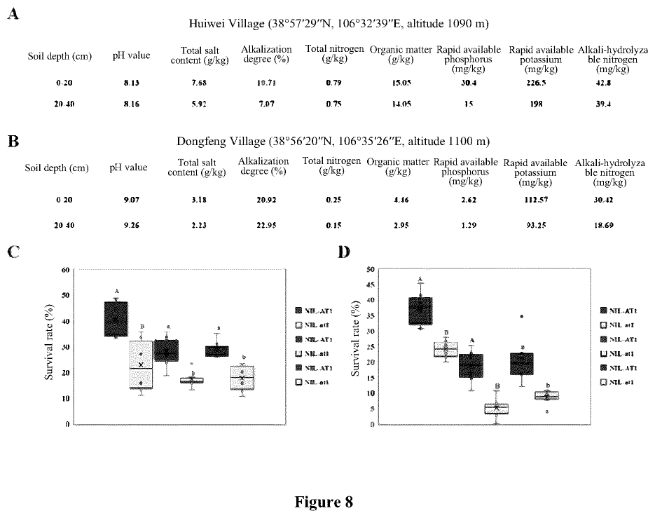

In order to fit the actual production, field regional trials were carried out in Huiwei Village (38° 57′29″N, 106° 32′39″E, altitude 1090 m) and Dongfeng Village (38° 56′20″N, 106° 35′26″E, altitude 1100 m) in Pingluo County, Shizuishan City, Ningxia Hui Autonomous Region, China from April to October 2020. The area is close to the Yellow River, with sufficient water resources and suitable for agricultural development. However, due to natural and human factors such as long-term flooding, high groundwater level, large evaporation and unreasonable fertilization, the secondary salinization of the soil in the irrigation area is serious, and agricultural cultivated land has also experienced varying degrees of secondary salinization and alkalization. A single-factor completely randomized block design was used. Three replicates were selected for each plot, with a total of six plots. The plot had a row length of 7 m, a row width of 5 m, a row spacing of 0.6 m, a plant spacing of 0.2 m, and a walkway of 0.8 m, and the plot area was 35 m². Double seed hole sowing was used, and the sowing depth was about 3 cm. Before sowing, seeds with full grains and uniform size were selected, and basal fertilizer (N:P₂O₅:K₂O=15:15:15) was applied, and the land was raked and leveled. Management projects such as intertillage, weeding and fertilization were carried out on time throughout the growth period. The physical and chemical properties of the test soil were shown in FIGS. 8A and B. Due to the uneven distribution of saline-alkali land and the existence of “alkali spots”, we found that the growth of sorghum in different plots was quite different, but the overall trend was consistent, all indicating that the growth state of NIL-AT1 was better than that of NIL-at1, and the statistical data of the seedling rate of the six plots showed that NIL-AT1 far exceeded NIL-at1 (P<0.05) (FIGS. 8C and D).

The method used in this example was as follows:

### (I) Construction of Sorghum AT1 Gene Overexpression Vector

The plant expression vector pCAMBIA2300-Ubi-Myc was preserved by out laboratory (see, Liu Y, Sun J, Wu Y. Arabidopsis ATAF1 enhances the tolerance to salt stress and ABA in transgenic rice. J Plant Res. 2016 September; 129 (5): 955-962. doi:10.1007/s10265-016-0833-0. Epub 2016 May 23. PMID: 27216423, wherein the vector was described in lines 1-3 of “Constructs and transformation” in “Materials and methods” on the right column of page 2, which was a recombinant vector with Ubi promoter and OCS terminator added to the commercial vector pCAMBIA2300 and with a Myc tag sequence, and was available to the public from the applicant to replicate the present invention). First, the vector was linearized by restriction endonucleases Spe I and BamH I (both purchased from New England Biolabs) on a PCR instrument for 2 h at 37° C. to obtain a linearized vector. Then, primers carrying adapters were used to amplify the CDS coding region (nucleotide sequence shown in SEQ ID NO: 3) of the sorghum AT1 gene with a stop codon to obtain the target fragment with adapters, and the obtained target fragment with adapters (gene fragment) and the obtained linearized vector were ligated using the seamless cloning kit pEASY-Uni Seamless Cloning and Assembly Kit (CU101-01). The system of the seamless cloning kit ligation reaction (10 μL) comprised: 5 μL of 2×Assembly Mix, the linearized vector and the target fragment with adapters which were mixed in a molar ratio of 1:2, and ddH₂O was added to make up to 10 μL. After gentle mixing, the reaction was carried out at 50° C. for 15 min. After the reaction was completed, the centrifuge tube was placed on ice and cooled for a few seconds. Then the recombinant product could be transformed into E. coli XL1-Blue, the plasmid were extracted, and the positive clone recombinant plasmid pCAMBIA2300-Ubi-Myc-AT1 could be confirmed by enzyme digestion, and sent to Ruibo Company for further sequencing verification.

### (II) Acquisition of AT1 Gene Overexpressing Plants

### Acquisition of Recombinant Agrobacterium

1 μg of the prepared recombinant vector plasmid pCAMBIA2300-Ubi-Myc-AT1 was taken and transformed into the competent cells of Agrobacterium EHA105. The positive bacteria identified by PCR were named recombinant Agrobacterium EHA105/pCAMBIA2300-Ubi-Myc-AT1, and stored at −80° C.

Recombinant Agrobacterium EHA105/pCAMBIA2300-Ubi-Myc-AT1 contained the CDS nucleotide sequence of sorghum AT1 gene (SEQ ID NO: 3).

### Acquisition of Plants Overexpressing Sorghum AT1 Gene

The recombinant Agrobacterium EHA105/pCAMBIA2300-Ubi-Myc-AT1 was transferred into the recipient material Wheatland of sorghum by Agrobacterium-mediated method.

The genetic transformation method for sorghum was as follows:

### (1) Preparation of Sorghum Recipient Material:

Wheatland sorghum plants were planted in a greenhouse. When they grew to about 15 days of flowering phase, the ears were cut off, and the immature embryos in grains were squeezed out as much as possible in a sterile clean bench and evenly placed on a callus medium for callus culture.

### (2) Preparation of Bacterial Solution for Infection:

The recombinant Agrobacterium stored at −80° C. was streaked and cultured in YEP solid medium supplemented with Kan. The single colony was inoculated into 10 mL of YEP liquid medium (containing Kan) for activation (shaking at 28° C., 220 rpm overnight). The activated bacterial solution was inoculated into 80 mL of YEP liquid medium (containing Kan) at a ratio of 1:1000, and cultured at 28° C., 220 rpm until the OD600 nm was 0.8 to 1.0.

### (3) Infection Process:

The Agrobacterium bacterial solution reaching the required OD value was transferred into a 50 mL centrifuge tube and centrifuged at 5000 rpm for 10 min to enrich the bacterial cells. Then the bacterial cells were fully resuspended with resuspension buffer, and the grown callus tissue was added to the resuspended infection solution for infection and transformation. The infected callus was transferred to a differentiation and regeneration medium. After regeneration, it was moved to a rooting medium (containing Kan) for rooting culture. If the medium browned, the rooting medium needed to be transferred multiple times.

(4) The transformed seedlings of TO generation sorghum with a plant height of about 5-7 cm and good root growth were transplanted into soil, and cultivated in a greenhouse, and a certain humidity was maintained to ensure the survival rate of the regenerated seedlings. After they are cultivated to maturity, the mature seeds were harvested, which were the TO generation transgenic seeds.

### (III) Construction of AT1 Gene Mutant Plants

### Construction of Recombinant CRISPR-Cas9 Vector

The AT1 gene in the sorghum recipient material variety Wheatland was knocked out using gene editing technology. The original vectors for monocot gene editing CRISPR-Cas9, including the gRNA expression cassette pYLsgRNA-OsU6a/LacZ vector and the CRISPR-Cas9 vector pYLCRISPR/Cas9Pubi-B, were kindly provided by Professor Liu Yaoguang from South China Agricultural University (Ma X, Zhang Q, Zhu Q, et al., A Robust CRISPR/Cas9 System for Convenient, High-Efficiency Multiplex Genome Editing in Monocot and Dicot Plants. Mol Plant. 2015 August; 8 (8): 1274-84. doi:10.1016/j.molp.2015.04.007. Epub 2015 Apr. 24. PMID: 25917172.).

### Acquisition of AT1 Gene Mutant Plants

First, the bacterial strain containing the pYLsgRNA-OsU6a/LacZ vector and the CRISPR-Cas9 vector pYLCRISPR/Cas9Pubi-B was activated and the plasmids were extracted. Next, the target adapter was prepared and the sgRNA vector was digested, and the ligation to the sgRNA expression cassette was performed. The nucleotide sequence of the sgRNA target was 5′-ACGCCTGAAAAGTTGACAGCTG-3′, targeting the positions 2317-2338 of SEQ ID NO: 1 of the AT1 gene. The manipulation method was specifically described as follows:

1) Activation of bacterial strain, preparation and extraction of plasmid: pYLCRISPR/Cas9Pubi-B-harboring strain (TOP10F) and pYLsgRNA-OsU6a/LacZ-harboring strain (DH10B) were streaked and cultured overnight on plates containing culture media containing kanamycin (25 μug/mL) and ampicillin (50 μg/mL), respectively, and single colonies were picked out for expansion culture for plasmid extraction.

2) Preparation of target adapter: The designed and synthesized adapter primers AT1-Target-F: 5′-GCCGCAAGTCGCCGCCTGCCTCGC-3′ and AT1-Target-R: 5′-AAACGCGAGGCAGGCGGCGACTTG-3′ were dissolved in ddH₂O to form a 100 μM stock solution, and the left and right primers were taken, mixed and diluted to a final concentration of 1 uM. Heating was performed on a PCR instrument (about 90° C. for 30 s), and cooled at room temperature to complete annealing to form a double-stranded target adapter.

3) Enzymatic digestion of sgRNA vector: 1 μg of pYLsgRNA-OsU6a/LacZ plasmid was taken, digested with 10 U Bsa I for 20 min in 25 μL of digestion reaction system to obtain the digested plasmid, which was stored under freezing condition.

4) sgRNA expression cassette ligation reaction: The digested plasmid and the corresponding double-stranded target adapter were subjected to ligation reaction (10 μL system): 1 μL of 10×T4 DNA ligase buffer, 10 ng of digested plasmid, 0.05 μM double-stranded target adapter, 18 U of T4 DNA ligase were supplemented with ultrapure water to make up to 10 μL; and ligation was carried out at room temperature for 20 min.

5) First round of amplification: in 15 μL of reaction system: 0.5 μL of the ligation product was taken as a template, forward primer U-F: 5′-CTCCGTTTTACCTGTGGAATCG-3′ and adapter reverse primer gRNA-R: 5′-CGGAGGAAAATTCCATCCAC-3′, 0.2 μM each, and 1 μL of KOD Fx Neo enzyme were used; and PCR program comprised: 30 cycles: 94° C. 10 s, 60° C. 15 s, 68° C. 20 s.

6) Second round of amplification: the first round of PCR product was diluted 10 times, and 1 μL was taken and used as a template for second round of PCR. An appropriate amount of KOD Fx Neo enzyme was also used, forward primer B1′ 5′-TTCAGAggtctcTctcgCACTGGAATCGGCAGCAAAGG-3′ and reverse primer BL: 5′-AGCGTGggtctcGaccgGGTCCATCCACTCCAAGCTC-3′ were used. Amplification was performed for 30 cycles: 95° C. 10 s, 58° C. 15 s, 68° C. 20 s. The size of the target band (831 bp) was detected by running gel, and purification was performed by ethanol precipitation to obtain the purified PCR product, that was the sgRNA with OsU6a promoter: OsU6a-sgRNA.

7) About 2 μg of pYLCRISPR/Cas9Pubi-B plasmid was taken and digested in an enzyme digestion system (30 U Bsa I) for 2 h, and then the digested plasmid fragment was recovered by DNA gel electrophoresis. 100 ng of pYLCRISPR/Cas9Pubi-B plasmid digested with Bsa I was taken, and added simultaneously with OsU6a-sgRNA (20 ng) which was the purified second round PCR product from step 6) and had been digested with Bsa I. 35 U of T4 ligase was added to 10 μL of ligation reaction system, and the ligation was performed for 2-3 h by variable temperature cycle: 10° C. 5 min, 20° C. 5 min; 10-15 cycles.

8) 1 μL of ligation product was taken and electroporated to E. coli DH10B competent cells. After electroporation, 1 mL of SOC was added and the cells were cultured at 37° C. for 2 h. The plating medium was LB (25 μg/mL Kan, 0.5 mM IPTG and appropriate amount of X-gal). A single clone colony was picked out and subjected to PCR amplification by colony PCR method and sequencing for specific target sites by using vector primers SP1: 5′-CCCGACATAGATGCAATAACTTC-3′ and SP2: 5′-GCGCGGTGTCATCTATGTTACT-3′. The amplified product had a band size of about 1 kb. After sequencing, the amplified product contained OsU6a-sgRNA. The obtained positive clone was the successfully recombined plasmid pYLCRISPR/Cas9Pubi-B-AT1-sgRNA.

9) 1 μg of the prepared recombinant vector plasmid pYLCRISPR/Cas9Pubi-B-AT1-sgRNA was taken and transformed to the competent cells of Agrobacterium EHA105, cultured in YEP medium (containing kanamycin 50 mg/L) at 28° C. for two days, the positive clone was picked out, and subjected to PCR identification using the above primers SP1 and SP2. The positive Agrobacterium identified by PCR was named recombinant Agrobacterium EHA105/pYLCRISPR/Cas9Pubi-B-AT1-sgRNA, and stored at −80° C.

The obtained positive Agrobacterium was used to infect the callus tissue of sorghum (Wheatland) and millet (Ci846). The genetic transformation procedure of sorghum was the same as the procedure 4.2.2. The millet recipient material Ci846 was kindly provided by Professor Sui Yi from the Institute of Crop Sciences, Chinese Academy of Agricultural Sciences (related literature: Cheng Z, Sun Y, Yang S, Zhi H, Yin T, Ma X, Zhang H, Diao X, Guo Y, Li X, Wu C, Sui Y. Establishing in planta haploid inducer line by edited SiMTL in foxtail millet (Setaria italica). Plant Biotechnol J. 2021 June; 19 (6): 1089-1091.). The genetic transformation of millet was carried out with reference to the method for genetic transformation of millet in the following patent document: Methods of obtaining embryonic callus tissue of millet for genetic transformation and of genetic transformation, publication number: CN108588002A.

The steps were specifically described as follows:

(1) Disinfection of mature embryos of millet: Mature embryos of millet seeds were disinfected in 10% sodium hypochlorite solution for 10-15 min, then rinsed repeatedly with sterile water for 3-5 times, and finally the water was absorbed with absorbent paper for later use.

(2) Induction of millet primary callus tissue: The callus tissue of mature embryos of seeds was induced in an induction medium and cultured in the dark at 28° C. for about 30 days.

(3) Preparation and large-scale expansion of millet embryonic callus cell suspension system: The induced primary callus tissue was transferred to a suspension culture medium for suspension culture, and the culture conditions were 28-30° C. and oscillation on a shaker with a speed of 150 r/min. During the culture process, the browned tissue was removed, and cells in better embryonic state were selected and expanded to obtain a large amount of embryonic cell callus tissue.

(4) Agrobacterium-mediated genetic transformation of millet embryonic cells: The recombinant Agrobacterium EHA105/pYLCRISPR/Cas9Pubi-B-AT1-sgRNA was activated in advance, the prepared callus tissue was co-cultured with the Agrobacterium in a pre-culture medium, and the co-cultured callus tissue was transferred to a screening medium containing the corresponding resistance for subculture. After three subcultures, the resistant calli were transferred to a differentiation and regeneration medium until seedlings grew out.

(5) The seedlings were transferred to a rooting medium for rooting culture. After growing a relatively strong root system, they were transplanted into soil and cultured in a greenhouse. In the late growth stage, positive seedlings were identified as transgenic TO generation seedlings, and TO generation seeds were harvested.

After obtaining the transgenic TO plants, the targeting effect was detected, that was, primers were synthesized at about 200-300 bp flanking the target site for PCR amplification. Internal primer was designed as a sequencing primer at about 150-250 bp upstream of the target site, and the PCR products were directly sequenced. If there were no overlapping peaks in the sequencing peak graph, it could be judged as wild-type or homozygous mutation. If there were double peaks appeared near the target site for a plant, the plant was a positive AT1 gene mutant plant.

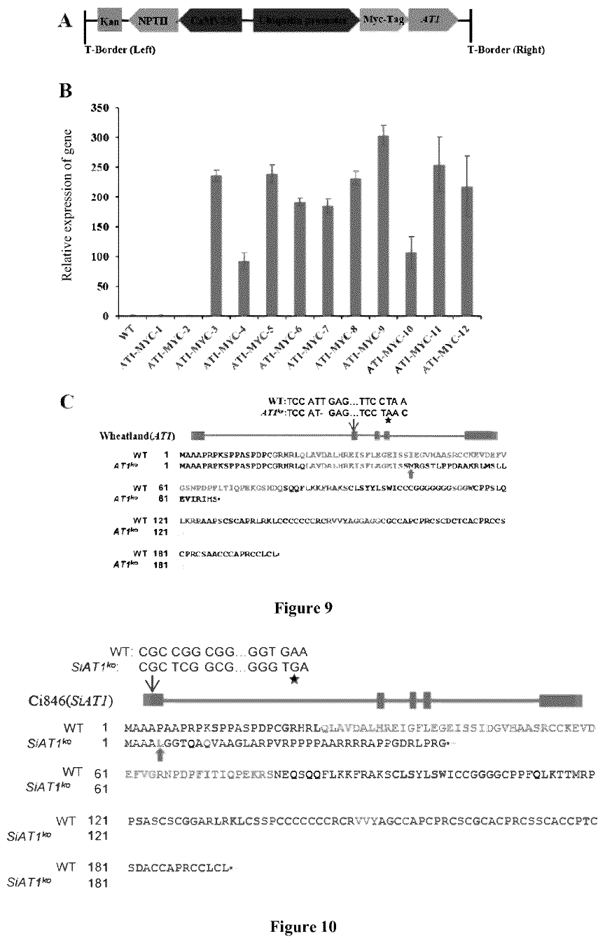

The genomic DNAs of the sorghum transgenic TO generation plant and the Wheatland (wild-type sorghum) were used as templates, respectively, the genomic DNA fragment comprising the splicing site was subjected to PCR amplification, and the amplified products were sequenced to detect the knockout of AT1 gene. It was previously predicted that the amino acids at positions 22-88 at the N-terminal of the AT1 protein in sorghum (i.e., the amino acids at positions 22-88 of SEQ ID NO: 4) were a highly conserved domain. CRISPR-Cas9 transgenic plants were subjected to PCR amplification using primers F: 5′-GTTGACAGCTGAACACATGGCT-3′ and R: 5′-ATACATCGTTAGGAATGGATCCG-3′, and the sequencing results showed that the AT1 gene was edited, resulting in a CRISPR-Cas9 transgenic plant with the loss of AT1 gene function. The transgenic plant with the edited AT1 gene was named AT1^{KO} (FIG. 9C). The results of alignment between the product of amplifying the AT1^{KO} plant with AT1 gene deletion using primers F and R and the product of amplifying the wild-type sorghum Wheatland using primers F and R showed that the AT1^{KO} plant with AT1 gene deletion lost one base T in the corresponding region of AT1 gene (the nucleotide at position 129 of SEQ ID NO: 3 was mutated), which caused the generation of a truncated protein in the corresponding region of the AT1 protein (the amino acid at position 43 of SEQ ID NO: 4 was mutated, resulting in the premature termination of subsequent translation), destroyed the conserved domain (FIG. 9C), thereby knocking out the AT1 gene. The coding sequence expressed by the AT1^{KO} plant was shown in SEQ ID NO: 11, and the amino acid sequence of the protein was shown in SEQ ID NO: 12.

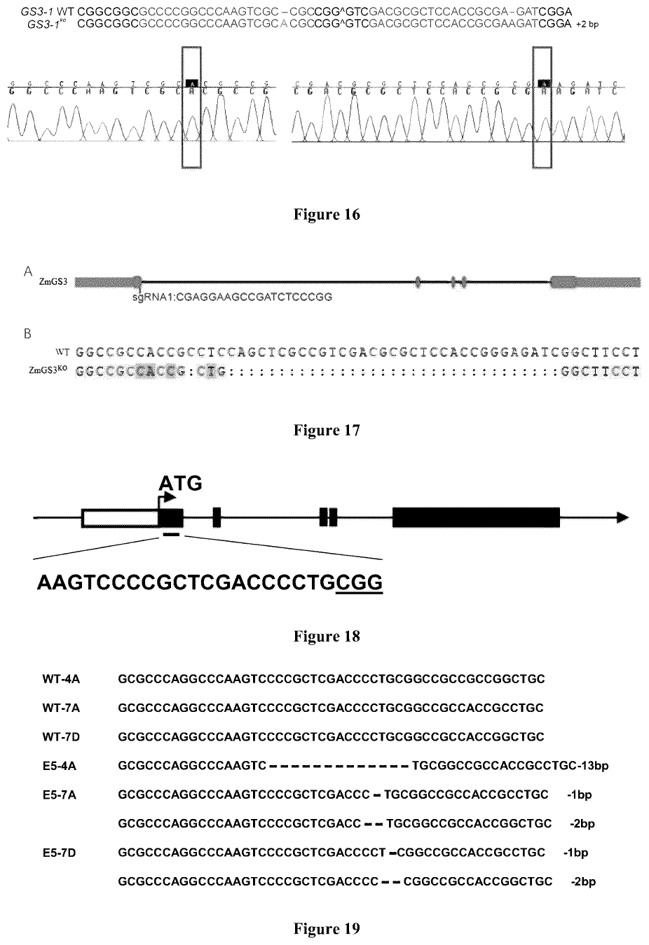

It was also predicted that the amino acids at positions 25-91 at the N-terminal of SiAT1 protein (SEQ ID NO: 8) in millet (the amino acids at positions 25-91 of SEQ ID NO: 8) also encode a conserved domain, and its corresponding coding sequence was shown in the nucleotide sequence at positions 73-273 of SEQ ID NO: 7. The mutant plant of millet SiAT1 obtained by the same gene editing method as above was named SiAT1^{KO} (FIG. 16C). In the millet SiAT1^{KO}, one base T was inserted between the 14^{th} and 15^{th} nucleotides of SEQ ID NO: 7 (wild-type SiAT1 gene), which caused a gene frameshift mutation, the production of a peptide segment of 42 amino acids, and the premature termination of protein translation. Therefore, the gene-edited material was a material in which the conserved domain of AT1 protein was completely deleted (FIG. 10). The coding sequence of AT1 expressed by the SiAT1^{KO} plant was shown in SEQ ID NO: 13, and the amino acid sequence of the protein was shown in SEQ ID NO: 14.

Among them, the nucleotide sequence of the sgRNA target of the millet SiAT1 gene was 5′-TATAATGGCTGCTGCGCCGG-3′, targeting the positions 393-412 of the SiAT1 gene (i.e., SEQ ID NO: 5); the adapter primers used in the preparation of the target adapter were SiAT1-Target-F: 5′-GGCGTATAATGGCTGCTGCGCCGG-3′ and SiAT1-Target-R: 5′-AAACCCGGCGCAGCAGCCATTATA-3′.

After the positive transgenic plants were obtained from screening in the TO generation of sorghum, the plants were selfed to obtain the T1 generation plants. When the T1 generation was harvested, the primers F and R were used again to identify target. After the PCR product was sequenced, the plant with target that was indeed edited and sequenced as single peak was retained, and used as a homozygous plant. The plant was then subjected to PCR amplification using the primers SP1: 5′-CCCGACATAGATGCAATAACTTC-3′ and SP2: 5′-GCGCGGTGTCATCTATGTTACT-3′. If no amplification product could be obtained, it was a homozygous plant that had Cas9 screened out, and this material could be used for subsequent experiments. The screened T1 generation homozygous plant was selfed to obtain a T2 generation homozygous plant.

Through gene editing technology, homozygous lines SbAT1^{ko} and SiAT1^{KO}, in which the CRISPR-Cas9 vector was screened out and the premature termination mutation was caused at the target site, were identified and screened in the T2 generations of transgenic sorghum and millet.

### Example 2. Application of Sorghum and Millet Resistant Plants Produced by Genetic Transformation of AT1 Gene

### Identification of Transgenic Sorghum and Millet Positive Plants and Observation of Saline-Alkali Tolerance Phenotype During Germination Stage

A total of 54 seedlings were obtained in the TO generation of AT1-MY C overexpressing transgenic sorghum, 12 TO transgenic positive plants were obtained by PCR identification. Then, by qPCR identification, it was observed that 10 plants (named AT1-MYC-3 to AT1-MYC-12) among them had AT1 gene expression levels much higher than that of the wild-type Wheatland, ranging from 92 to 303 folds (FIG. 9B), with SbEIF as an internal reference gene. Next, in order to select an appropriate concentration to identify the alkali tolerance of the successfully constructed transgenic sorghum and millet, three alkali concentration gradients (50 mM mixed alkali solution, 75 mM mixed alkali solution and 100 mM mixed alkali solution) were set, and a control (water) treatment was set. The seeds used for the experiment were all grown under the same conditions, harvested at the same time and stored under the same conditions. The seeds were sown in a soil matrix of vermiculite and nutrient soil mixed in a ratio of 1:1. 12 seeds were sown in each hole, and then covered with a soil layer of about 2 cm, which was leveled with the surface of the trays, and two replicates were set. Then, the soil was irrigated with the treatment solution (water or alkaline solution) until saturated as treatment group, and irrigated with clean water as the control group, and the trays were placed on a flat ground for sufficient and even absorption in the soil, and then the trays were placed in a model plant glass greenhouse (the seedling growth environment was as follows: the day/night temperature was controlled at 28° C./22° C., the light/dark time was 16 h/8 h, and the relative humidity was 60% to 70%); and clean water was replenished in the later stage, and three replicates were set for each treatment group. When the plantules of the seeds topped out of the soil surface, the seeds were considered to have germinated. Among them, survival rate=number of seedling seeds (14 days)/number of test seeds×100%; relative survival rate=survival rate of alkali treatment/survival rate of corresponding control×100%. The statistical method was as follows: Excel 2016 was used first for data sorting and chart making, DPS 7.5 software was used for data statistics, and the least significant difference method (LSD) and one-way analysis of variance (ANOVA) were used to perform significant variance analysis on the results. If P<0.05, it was marked with lowercase letters, and the difference was significant; if P<0.01, it is marked with uppercase letters, and the difference was extremely significant.

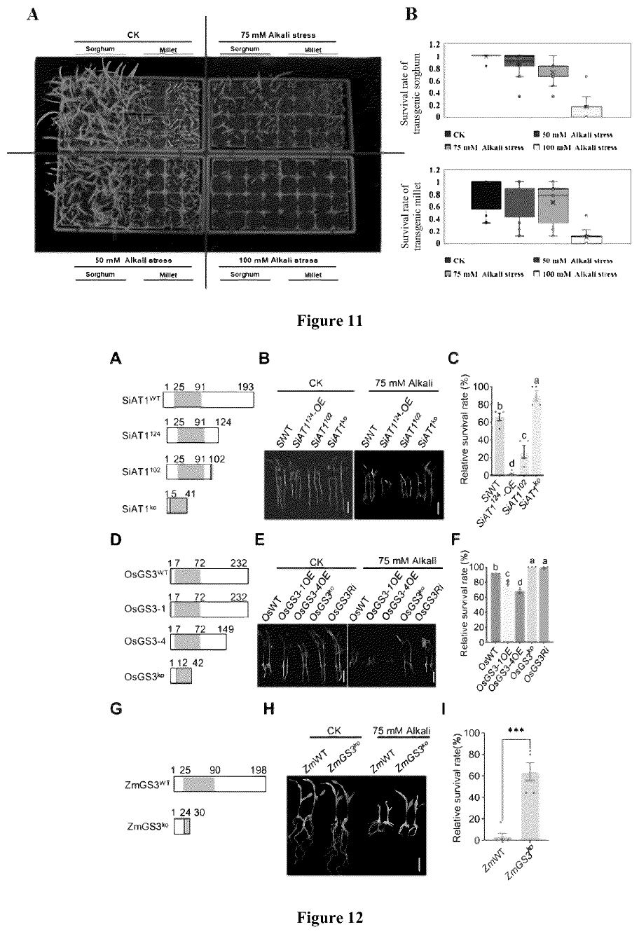

By observing the growth after alkali treatment and analyzing the relative seedling rates, we believed that the phenotypic variation was the highest when the 75 mM mixed alkali treatment was used, and it was suitable to select this concentration for later phenotypic identification (FIG. 11). In FIG. 11, Sorghum represents sorghum group; Millet represents millet group.

Among them, the preparation method of 50 mM mixed alkali solution (NaHCO₃:Na₂CO₃ in with molar ratio of 5:1) comprised: 12.32 g of NaHCO₃ and 3.11 g of Na₂CO₃ were weighed and dissolved in 2 liters of water, the solution pH was 10.03, and the soil pH was 9.19 after the soil fully absorbed the solution; the preparation method of 75 mM mixed alkali solution (NaHCO₃:Na₂CO₃ with a molar ratio of 5:1) comprised: 18.48 g of NaHCO₃ and 4.66 g of Na₂CO₃ were weighed and dissolved in 2 liters of water, the solution pH was 10.04, and the soil pH was 9.32 after the soil fully absorbed the solution; the preparation method of 100 mM mixed alkali solution (NaHCO₃:Na₂CO₃ with a molar ratio of 5:1) comprised: 24.64 g of NaHCO₃ and 6.22 g of Na₂CO₃ were weighed and dissolved in 2 liters of water, the solution pH was 10.03, and the soil pH was 10.10 after the soil fully absorbed the solution.

The results of phenotypic observation showed that under normal growth conditions of the control (CK), there was no significant difference in survival rates among the wild-type sorghum Wheatland, the sorghum AT1 gene overexpression line AT1-OE and the sorghum AT1 gene deletion mutant line AT1^{KO} (FIG. 5H, FIG. 6E), and there was no significant difference in survival rates among the wild-type millet Ci846 (SiWT in FIG. 12B), the millet SiAT1 gene overexpression line SiAT1-OE and the deletion mutant line SiAT1^{KO} (FIG. 12B). Under the 75 mM mixed alkali treatment condition (the soil pH value was 9.32), the sorghum AT1-OE line was extremely sensitive to alkali, while the sorghum mutant AT1^{KO} and millet mutant SiAT1^{KO} with loss of gene function both showed increased saline-alkali tolerance as compared with the wild-type sorghum Wheatland and millet Ci846 (FIG. 6E-F and FIG. 12B-C). The above results all indicated that SbAT1 of sorghum and SiAT1 of millet were indeed important genes that negatively regulated the saline-alkali tolerance of sorghum and millet, and the mutation of their N-terminal GGL conserved domain (SEQ ID NO: 15) helped to improve the saline-alkali tolerance of sorghum and millet.

In addition, the results in Examples 1 and 2 had shown that, relative to the wild-type AT1 protein (SEQ ID NO: 1) with 198 amino acids, the at1 (SEQ ID NO: 10) in the NIL-at1 mutant that had truncation at the C-terminal and retained only 136 amino acids was the protein corresponding to the natural variant allele of AT1. Analysis of near-isogenic line accessions showed that as compared with the wild-type AT1, the alkali tolerance of the at1 that lost the C-terminal but retained the complete N-terminal GGL domain was greatly reduced. Therefore, all the above results in Examples 1 and 2 indicated that the AT1 protein in Graminede crops sorghum and millet was a key factor in negatively regulating the alkali tolerance, and the saline-alkali tolerance of crops could be improved by gene-editing of the AT1 gene or by knocking out AT1.

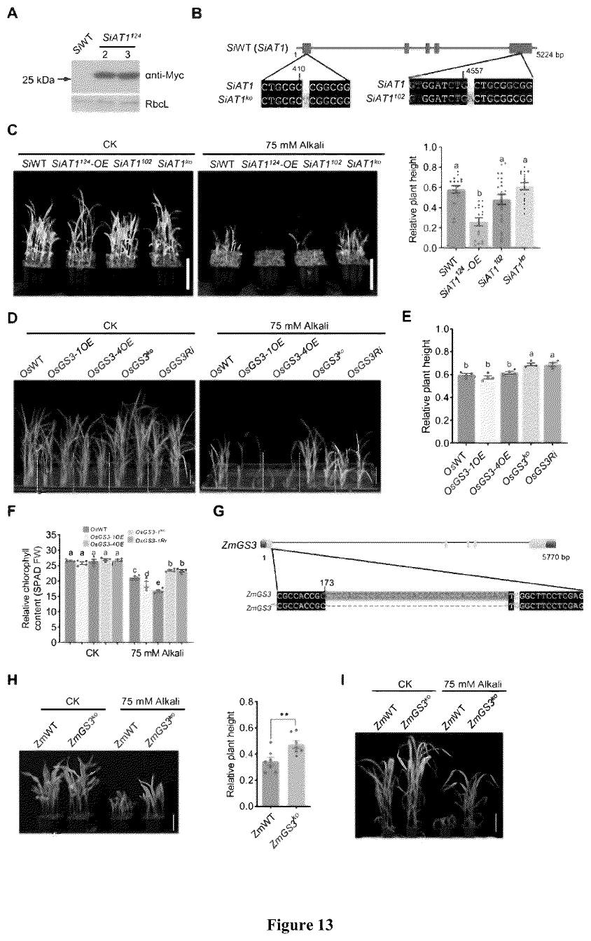

To further confirm the above observation, we produced the following transgenic plants: a stop codon was generated at the same position as sorghum by gene editing to mimic the potential C-terminal truncated SiAT1¹⁰² protein in millet. We also produced plants overexpressing SiAT1¹²⁴ with a truncated C-terminal (SiAT1¹²⁴-OE) and plants with SiAT1 knockout (SiAT1^{KO}) (FIG. 12A, FIG. 13A-B).

Millet was used for testing in this example based on the following considerations: (1) plant transformation of millet was easier and faster than that of sorghum, (2) millet and sorghum were closely related in the taxonomy, and had high genomic similarity and similar environmental physiological performance, and (3) the single copy millet SiAT1 shared 75.24% identity with sorghum SbAT1 at the protein level.

Similar to the results of transgenic sorghum, knockout of the SiAT1 gene resulted in plants with higher tolerance to alkaline conditions, as shown by the higher survival rates observed in SiAT1^{KO} plants compared with other genotypes (FIG. 12B-C). In contrast, C-terminal truncated transgenic millet (SiAT1¹⁰²) and millet plants overexpressing C-terminal truncated SiAT1 (SiAT1¹²⁴-OE) showed a reduced alkali tolerance under alkaline stress treatment, with SiAT1¹²⁴-OE plants showing the weakest growth response to alkalinity (FIGS. 12B-C, FIG. 13C). This result suggested that C-terminal truncated proteins could be expressed in plants and that increased amounts of C-terminal truncated proteins had a negative effect on alkali tolerance, while knocking out AT1 had a positive effect on alkali tolerance in plants. In conjunction with the AT1 overexpression phenotype observed in sorghum, we were able to conclude that AT1 exhibited a negative regulatory function in alkali tolerance in sorghum and millet, and that at1 mutations enhanced this negative regulatory function.

### Example 3. Similar Roles of AT1 Homologous Genes in Rice and Maize

We further investigated the role of AT1 homologous genes in alkali tolerance in other major monocot crops, rice and maize. The ortholog of AT1 in rice had been identified as OsGS3, which was a major QTL for grain size (H. Mao, S. Sun, J. Yao, C. Wang, S. Yu, C. Xu et al., Linking differential domain functions of the GS3 protein to natural variation of grain size in rice. Proc. Natl. Acad. Sci. USA 107, 19579-19584 (2010). doi:10.1073/pnas. 1014419107; C. Fan, Y. Xing, H. Mao, T. Lu, B. Han, C. Xu et al., GS3, a major QTL for grain length and weight and minor QTL for grain width and thickness in rice, encodes a putative transmembrane protein. Theor. Appl. Genet. 112, 1164-1171 (2006). doi:10.1007/s00122-006-0218-1).

The rice GS complete protein (GS3-1) sequence was shown in SEQ ID NO: 16, the rice GS3 C-terminal truncated protein (GS3-4) sequence was shown in SEQ ID NO: 17, and the maize GS3 protein sequence was shown in SEQ ID NO: 18.

We found that in alkaline soil (75 mM mixed alkali, pH 9.0 to 9.2), overexpression of intact OsGS3 (OsGS3-10E) and C-terminally truncated version of OsGS3 (OsGS3-40E) showed lower alkali tolerance, while rice with knockout of OsGS3 (OsGS3^{ko}) or RNAi OsGS3 (OsGS3Ri) showed higher alkali tolerance than ZH11 wild-type rice (OsWT) in terms of relative survival rate, and plant growth measured by relative plant height and relative chlorophyll content (FIG. 12D-F and FIG. 13D-F). In transgenic plants, the relative survival rates of OsGS3-10E and ( ) GS3-4OE were 12.5% and 26.4% lower than that of OsWT, respectively, while the relative survival rates of OsGS3^{ko} and OsGS3Ri were 8.3% and 7.4% higher than that of OsWT, respectively (FIG. 12F). These results indicated that inhibition of rice OsGS3 function could enhance alkali tolerance in rice, and suggested that Gγ subunit had a conserved function. In addition, by manipulating or selecting non-functional alleles of OsGS3, we could enhance alkali tolerance in rice.

The maize homologous gene of AT1 was previously identified as ZmGS3 (Q. Li, X. Yang, G. Bai, M. L. Warburton, G. Mahuku, M. Gore et al., Cloning and characterization of a putative GS3 ortholog involved in maize kernel development. Theor. Appl. Genet. 120, 753-763 (2010). doi:10.1007/s00122-009-1196-x). Therefore, we named the gene AT1/GS3 with a prefix indicating the species; AT1 and GS3 could also be used separately or interchangeably depending on the context. The maize ZmGS3 knockout (ZmGS3^{ko}) line was obtained by gene editing of the maize line KN5585 (ZmWT). Maize ZmGS3^{ko} had a deletion of 34 bp and base mutation in the first exon of ZmGS3. These mutations leaded to a frameshift mutation, and premature translation termination of the predicted protein (FIG. 13G). After alkaline treatment, the growth performance of the knockout maize plants on the 14^{th} day of culture showed that they had stronger alkali tolerance as compared with the wild-type maize (FIG. 12G-H, FIG. 13H). After 50 days of alkaline stress treatment, the phenotypic difference between the two lines was more significant; almost all wild-type maize seedlings died, while ZmGS3^{ko} survived and continued to grow (FIG. 12I, FIG. 13I). This result supported that ZmGS3^{ko} could improve alkali tolerance in maize, similar to what we observed in sorghum, millet, and rice.

The method used in this example was described as follows:

### Study on the Function of GS3 Gene in Regulating Alkali Tolerance

### 3.1 Construction of Transgenic Plants Overexpressing GS3 Gene

Primers were designed based on the rice genome annotation information (GS3 genomic sequence as shown in SEQ ID NO: 19), and the full-length cDNA of GS3 of rice variety Guangluai (Osigcea013f09t3) was used as a template to amplify two alleles of GS3, i.e., GS3-1 and GS3-4, with sizes of 696 bp (as shown in SEQ ID NO: 20) and 450 bp (as shown in SEQ ID NO: 21), respectively.

In order to amplify the cDNA of GS3 gene coding region, the following primers were designed in the present invention:

GS3OEF forward primer (SEQ ID NO: 36):
5′-ggtaccACCATGGCAATGGCGGCGGCGCCC-3′ (the
underlined sequence was the KpnI recognition
site);
GS3-1OER reverse primer (SEQ ID NO: 37):
5′-agatctCAAGCAGGGGGGGCAGCAACG-3′ (the underlined
sequence was the BglII recognition site);
GS3-4OER reverse primer (SEQ ID NO: 38):
5′-agatctACGCCGCCCCACATGAGGA-3′ (the underlined
sequence was the BglII recognition site).

The primer combinations GS3OEF, GS3-10ER and GS3OEF, GS3-40ER were used for PCR amplification to obtain the target fragments of GS3-1 and GS3-4, respectively. The total volume of the PCR reaction was 50 μl, comprising 2 μl of cDNA template, 25 μl of 2×GC I buffer, 5 μl of 10 mM dNTPs, 1 μl of each of 10 mM primers GS3OEF and GS30ER, 1 μl of ExTaq enzyme, and deionized water supplemented to 50 μl (the 2×GC I buffer, dNTPs, and ExTaq enzyme used herein were purchased from Takara Biotech Dalian Co., Ltd.). The PCR reaction conditions were as follows: \textcircled{1} 94° C. 4 min, \textcircled{2} 94° C. 30 s, \textcircled{3} 58° C. 30 s, \textcircled{4} 72° C. 1 min, \textcircled{5} 33 cycles from \textcircled{2} to \textcircled{4}, \textcircled{6} 72° C. 7 min, \textcircled{7} 25° C. storage. The PCR products were detected by electrophoresis on 1% (mass/volume) TBE agarose gel, and DNA fragments of GS3-1 with length of 696 bp (681 bp target DNA segment plus two 15 bp restriction enzyme sites attached to the primers) and 462 bp GS3-4 (447 bp target DNA segment plus two 15 bp restriction enzyme sites attached to the primers) were recovered. They were constructed into the commonly used overexpression vector pCAMBIA1301U (this vector was modified in our laboratory: its basic skeleton was pCAMBIA1301 of the Australian CAMBIA laboratory (http://www.cambia.org/daisy/cambia/materials/overview.html), and the expression regulation of the transformed gene was achieved by incorporating Ubi promoter), and the rice Zhonghua 11 (ZH11) was transformed to obtain overexpression plants GS3-10E and GS3-40E.

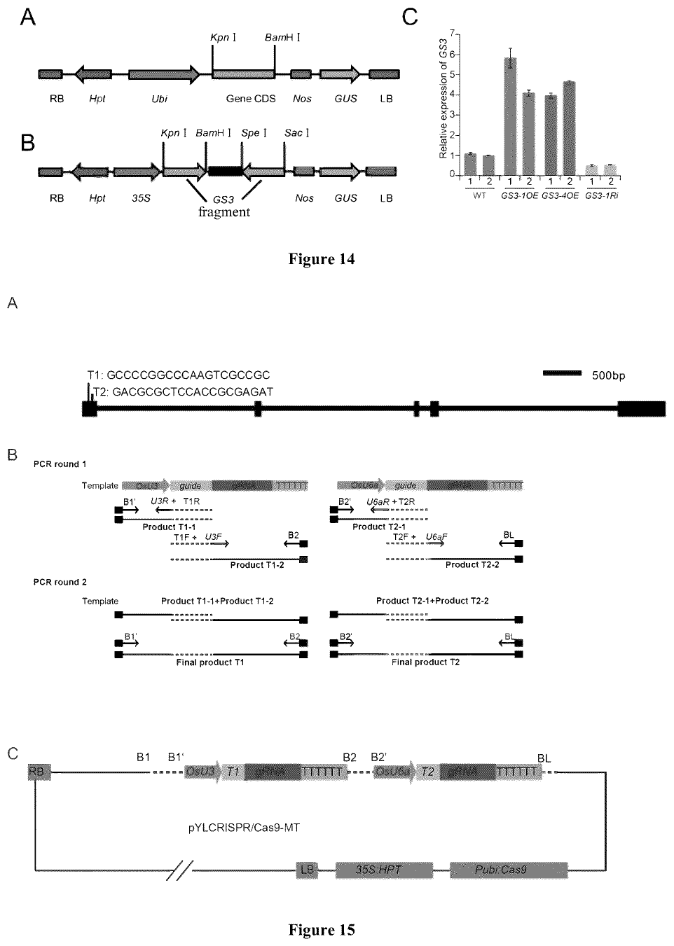

The vector structure was shown in FIG. 14A.

### 3.2 Construction of RNAi Interference Plants

Construction of RNAi vector for inhibition of GS3 expression was carried out in two steps. First, the plasmid containing (S3 full-length cDNA (osigcea013f09t3) was double-digested with BamHI and KpnI, and ligated to the modified dsRNAi1301 to obtain a vector containing the first-chain (forward) GS3; at the same time, the digestion product of BamHI and KpnI was ligated to an intermediate vector GZ-1 (provided by Dr. Ding Xinhua from our laboratory), and then digested with SacI and SpeI enzymes, and then the product was ligated to the dsRNAi1301 containing the first chain (also digested with SacI and SpeI), so that the two fragments of the same sequence were in opposite directions, driven by 35S promoter, and transformed into Zhonghua11 to obtain a transgenic plant GS3-1RNAi with suppressed expression. The schematic presentation of the vector structure was shown in FIG. 14B.

By the method of knocking down the expression of GS3 gene or protein in plants using RNA interference, the coding sequence of plant GS3 gene was preferably inserted into pDS1301 in the forward and reverse directions, respectively, and the obtained recombinant vector was transformed into a plant to obtain a plant with reduced expression of the GS3 gene or protein. The method of inserting the GS3 gene into the pDS1301 vector was preferably completed by enzyme digestion. When inserting in the forward direction, KpnI and BamHI double-enzyme digestion and ligation were adopted. When inserting in the reverse direction, SacI and SpeI double-enzyme digestion and ligation were adopted. When the coding sequence of the GS3 gene was inserted in the forward direction, the nucleotide sequence was shown in SEQ ID NO: 34, and when the coding sequence of the GS3 gene was inserted in the reverse direction, the nucleotide sequence was shown in SEQ ID NO: 35. Among them, the pDS1301 vector was modified by our laboratory (see, the prior art: Yuan B, Shen X, Li X, Xu C, Wang S (2007) Mitogen-activated protein kinase OsMPK6 negatively regulates rice disease resistance to bacterial pathogens. Planta 226:953-960); its basic framework was pCAMBIA1301 from the Australian CAMBIA laboratory (http://www.cambia.org/daisy/cambia/materials/overview.html), and the expression of the transformed gene was regulated by incorporating 35S promoter.

### 3.3 Expression Level Detection of Overexpression and Suppressed Expression in Transgenic Plants

(1) RNAs were extracted from 1 cm long spikelets of GS3-10E, GS3-40E and GS3-1RNAi as well as wild-type plants at the spikelet differentiation stage. The reagents used for RNA extraction were those of the Trizol extraction kit produced by Invitrogen (the specific operation steps were carried out according to the instructions provided by the kit).

(2) Synthesis of cDNA first strand by reverse transcription

The steps were as follows:
\textcircled{1} 3 μg of extracted total RNA was taken, added with 1 μl of DNaseI, 1 μl of 10× DNaseI buffer, and added with DEPC (diethyl pyrocarbonate, a strong inhibitor of RNase, working concentration was 0.01%) treated water to make up 10 μl, mixed well and left at room temperature for 15 min to remove residual genomic DNA;
\textcircled{2} 1 μl of 0.2 M EDTA was added, and incubated in a 65° C. water bath for 10 min to remove the activity of DNaseI;
\textcircled{3} 1 μl of oligo (dT)₁₅ primer was added and incubated in a 65° C. water bath for 10 min to destroy the secondary structure of RNA, and then placed on ice for 2 min;
\textcircled{4} 4 μl of 5× first strand buffer, 2 μl of 0.1 M DTT (dithiothreitol), 1 μl of 10 mM dNTP mixture, and 1 μl of reverse transcriptase were added, mixed well and placed in a 42° C. water bath for 1.5 h;
\textcircled{5} after the reaction was completed, the reverse transcription product was placed in an 85° C. dry bath for 10 min to inactivate the reverse transcriptase;
\textcircled{6} 80 μl of water was added to the reverse transcription product, mixed well and stored as the final product at −20° C. All reagents used in the reaction were purchased from Invitrogen.

(3) The obtained reverse transcription product was detected by real-time PCR. The GS3 gene detection primers were GS3QRT-F and GS3QRT-R, and the Ubiquitin gene (LOC_Os03g13170) was used as an internal reference (the primer combination was UbiQRT-F and UbiQRT-R). The sequences were as follows:

GS3QRT-F:
(SEQ ID NO: 39)
5′-CCGCGAGATCGGATTCC-3′;
GS3QRT-R:
(SEQ ID NO: 40)
5′-CGTGGATCCCTTCGATTGA-3′;
UbiQRT-F:
(SEQ ID NO: 41)
5′-AACCAGCTGAGGCCCAAGA-3′;
UbiQRT-R:
(SEQ ID NO: 42)
5′-ACGATTGATTTAACCAGTCCATGA-3′.

The reaction was carried out in a 10 μL system, which contained 1 μL of reverse transcription product, 0.3 μL of each of forward and reverse primers, and 5 μL of FastStart Universal SYBR Green Master, and water was added to make up 10 μL. The reaction procedure was: \textcircled{1} 95° C. 10 min, \textcircled{2} 95° C. 10 s, \textcircled{3} 60° C. 30 s, \textcircled{4} 40 cycles from \textcircled{2} to \textcircled{3}. The relative expression was calculated based on the CT value.

The real-time PCR detection results showed that the expression levels of the target gene in rice plants GS3-10E and GS3-40E were significantly increased as compared with the wild-type ZH11. The expression level of the target gene in GS3RNAi plants was significantly decreased as compared with the wild-type ZH11, as shown in FIG. 14C.

### 3.4 Alkali Tolerance Regulation Function Experiment of GS3 Gene

The transgenic plants overexpressing the GS3 gene and the plants with reduced GS3 expression prepared above were treated with alkali, respectively, and their differences in the main agronomic traits were compared with non-transgenic recipients. The details were as follows: an alkali treatment experiment was conducted using seeds grown under the same conditions, harvested at the same time, and stored under the same conditions.

The seeds were sown in a soil matrix formed by vermiculite and nutrient soil in equal volume ratios, 12 seeds were sown in each hole, and then covered with a soil layer of about 2 cm, which was leveled with the surface of trays, and two replicates were set. Then, the soil was irrigated with the treatment solution (water or 75 mM mixed alkali solution) until saturated as treatment group, and irrigated with clean water as the control group, and the trays were placed on a flat ground for sufficient and even absorption in the soil, and then the trays were placed in a model plant glass greenhouse (the seedling growth environment was as follows: the day/night temperature was controlled at 28° C./22° C., the light/dark time was 16 h/8 h, and the relative humidity was 60% to 70%); and clean water was replenished in the later stage, and three replicates were set for each treatment group. When the embryos of the seeds topped out of the soil surface, the seeds were considered to have germinated. The survival rate and relative survival rate of the plants were calculated according to Formula I and Formula II:

$$Survival rate = number of seedling seeds ( 14 days ) / number of test seeds \times 100 \% \qquad ( Formula I )$$

$$Relative survival rate = survival rate of alkali treatment / survival rate of corresponding control \times 100 \% . \qquad ( Formula II )$$

The statistical method was as follows: Excel 2016 was used first to organize data and make charts, and DPS 7.5 software was used for data statistics. The least significant difference method (LSD) and one-way analysis of variance (ANOVA) were used to perform significant variance analysis on the results. If P<0.05, it was marked as lowercase letters to indicate significant differences; and if P<0.01, it was marked with uppercase letters to indicate extremely significant differences.

Phenotypic observation results showed that under normal water treatment, there was no significant difference in survival rates between the wild-type ZH11, the GS3 gene overexpression lines GS3-10E, GS3-40E, and GS3 suppressed expression plant GS3RNAi. In contrast, under 75 mM mixed alkali solution treatment, the survival rates of GS3-10E and GS3-40E transgenic plants decreased by 12.5% and 26.4% respectively compared with ZH11, while the survival rate of GS3 RNAi plant increased by 7.4% compared with ZH11 (FIG. 12F). The above results all indicated that the GAS3 of rice was an important gene that negatively regulated alkali tolerance.

The results showed that after alkaline stress treatment, the alkali tolerance of the overexpression transgenic rice decreased, while the suppression of GS3 significantly improved the alkali tolerance, indicating that GS3 was a previously undiscovered alkali negative regulatory gene. Therefore, inhibiting protein expression could improve alkali tolerance in plants.

### 3.5 Using CRISPR/Cas9 Gene Editing to Inhibit the Expression of GS3 Protein in Rice

A CRISPR/Cas9-based target site was designed for the rice GS3 gene, a sgRNA sequence was synthesized, and a DNA fragment containing the sgRNA sequence was ligated to a vector carrying CRISPR/Cas (for vector information, see: Ma X, Zhang Q, Zhu Q, et al. A Robust CRISPR/Cas9 System for Convenient, High-Efficiency Multiplex Genome Editing in Monocot and Dicot Plants. Mol Plant, 2015, 8 (8): 1274-84).

Two target sites were designed (as shown in SEQ ID NOs: 24 and 25), both located on the first exon of GS3 (FIG. 15A). The construction method of the gene editing vector pYL-Cas9-gRNA-GS3 was specifically described as follows:

### (1) Construction of OsU6a-T1-gRNA-polyT and OsU6b-T2-gRNA-polyT Fragments

In the first round PCR, pYL-U6a-gRNA plasmid was used as a template, and primers B1′ and T1R were used to amplify OsU6a promoter and GS3 gene 20 bp T1 target sequence; similarly, pYL-U3-gRNA was used as a template, and primers TIF and B2 were used to amplify GS3 gene T1 target sequence and gRNA-polyT; in the second round PCR, the first round PCR product was used as a template, and primers B1′ and B2 were used to amplify OsU6a-T1-gRNA-polyT fragment; and OsU6a-T2-gRNA-polyT fragment was obtained by the same method. In the first round PCR, the pYL-U6a-gRNA plasmid was used as a template, and primers B2′ and T2R were used to amplify OsU6b promoter and GS3 gene 20 bp T2 target sequence; similarly, pYL-U6b-gRNA was used as a template, and primers T2F and BL were used to amplify GS3 gene T2 target sequence and gRNA-polyT; in the second round PCR, the first round PCR product was used as a template, and primers B2′ and BL were used to amplify OsU6a-T2-gRNA-polyT fragment (FIG. 15B).

The primer sequences used in step 1) were as follows:

B1′:
TTCAGAggtctcTctcgCACTGGAATCGGCAGCAAAGG-3 (the underlined part was
BsaI restriction site, SEQ ID NO: 43)
B2:
AGCGTGggtctcGtcagGGTCCATCCACTCCAAGCTC-3 (the underlined part was BsaI
restriction site, SEQ ID NO: 44)
B2′:
TTCAGAggtctcTctgaCACTGGAATCGGCAGCAAAGG-3 (the underlined part was
BsaI restriction site, SEQ ID NO: 45)
BL:
AGCGTGggtctcGaccgGGTCCATCCACTCCAAGCTC-3 (the underlined part was
BsaI restriction site, SEQ ID NO: 46)
T1F:
AACGGATTCAGCCGGTCTCGGTTTTAGAGCTAGAAATAGCA (the underlined
part was target T1, SEQ ID NO: 47)
T1R:
CGAGACCGGCTGAATCCGTTTGCCACGGATCATCTGCACA (the underlined
part was target T1, SEQ ID NO: 48)
T2F:
GGGACTTGAACGGATTCAGCGTTTTAGAGCTAGAAATAGCA (the underlined
part was target T2, SEQ ID NO: 49)
T2R:
GCTGAATCCGTTCAAGTCCCCGGCAGCCAAGCCAGCACCCG (the underlined
part was target T2, SEQ ID NO: 50).

The total volume of the PCR reaction was 50 μl, containing 2 μl of cDNA template, 25 μl of 2×GC I buffer, 5 μl of 10 mM dNTP, 1 μl of each of 10 mM primers GS3OEF and GS30ER, 1 μl of ExTaq enzyme, and deionized water was added to make up 50 μl (the 2×GC I buffer, dNTP, ExTaq enzyme, etc. used herein were purchased from Takara Biotechnology Dalian Co., Ltd.); the PCR reaction conditions were as follows: \textcircled{1} 94° C. 4 min, \textcircled{2} 94° C. 30 s, \textcircled{3} 58° C. 30 s, \textcircled{4} 72° C. 1 min, \textcircled{5} 33 cycles from \textcircled{2} to \textcircled{4}, \textcircled{6} 72° C. 7 min, \textcircled{7} 25° C. storage.

(2) Construction of pYL-Cas9-gRNA-OsGS3

The PCR fragments of OsU6a-T1-gRNA-polyT and OsU6b-T2-gRNA-polyT as well as pYLCRISPR/Cas9-MT vector were subjected to a cutting-and-ligation method via BsasI, so as to ligate OsU6a-T1-gRNA-polyT and OsU6b-T2-gRNA-polyT to pYLCRISPR/Cas9-MT vector (FIG. 15C), thereby obtaining pYL-Cas9-gRNA-OsGS3 vector, which was transformed to Zhonghua 11 to obtain a transgenic plant OsGS3^{ko}.

### 3.6 Detection of Mutation of OsGS3^{ko}

Primers were designed at 111 bp upstream and 72 bp downstream of a target site, and the DNA of the transgenic plant OsGS3^{ko} obtained in Example 2 was subjected to PCR amplification, and the amplification fragments were sequenced to determine the mutation of the target site. GS3CRJCF/GS3CRJCR primers were used to perform PCR amplification, the primer sequences for identifying the mutation of GS3 were described as follows, and the fragment size was 273 bp.

GS3CRJCF:
(SEQ ID NO: 51)
TACATAGCTGCTGCACCGTC;
GS3CRJCR:
(SEQ ID NO: 52)
GAAGCAAGATCGAAGGAGTATG.

The total volume of the PCR reaction was 20 μl, containing 2 μl of DNA template, 20 μl of 2×GC I buffer, 2 μl of 2 mM dNTP, 0.2 μl of each of 10 mM primers GS3OEF and GS30ER, 0.2 μl of rTaq enzyme, and deionized water was added to make up 20 μl (the 2×GC I buffer, dNTP, rTaq enzyme, etc. used herein were purchased from Takara Biotechnology Dalian Co., Ltd.); the PCR reaction conditions were as follows: \textcircled{1} 94° C. 4 min, \textcircled{2} 94° C. 30 s, \textcircled{3} 58° C. 30 s, \textcircled{4} 72° C. 30 s, \textcircled{5} 33 cycles from \textcircled{2} to \textcircled{4}, 6̅ 72° C. 7 min, \textcircled{7} 25° C. storage. The sequencing results showed that a homozygous mutant OsGS3^{ko} was obtained in the present invention. The OsGS3^{ko} had a 2 bp insertion (see FIG. 16), and a sequence as shown in SEQ ID NO: 30.

A similar method was used to inhibit the GS3 protein (SEQ ID NO: 18) in maize inbred line KN5585 (application number for variety right: 20191002444), wherein the maize GS3 genomic sequence was shown in SEQ ID NO: 22, and had a cDNA sequence as shown in SEQ ID NO: 23. The target site was also designed at the first exon and had a sequence as shown in SEQ ID NO: 26, and the synthesized sgRNA sequence was shown in SEQ ID NO: 29. After the editing vector construction and maize genetic transformation were completed, primers were designed at 118 bp upstream and 113 bp downstream of the target site, and PCR amplification and sequencing were performed to determine the mutation of the target site. The primer sequences for identifying the mutation effect of ZmGS3 were as follows, and the fragment size was 255 bp.

ZmGS36F:
(SEQ ID NO: 53)
ACTATAACAATCGACGACGTG;
ZmGS36R:
(SEQ ID NO: 54)
AGCAGTGCAGCGTAATCGAT.

The total volume of the PCR reaction was 20 μl, containing 2 μl of DNA template, 20 μl of 2×GC I buffer, 2 μl of 2 mM dNTP, 0.2 μl of each of 10 mM primers ZmGS36F and ZmGS36R, 0.2 μl of rTaq enzyme, and deionized water was added to make up 20 μl (the 2×GC I buffer, dNTP, rTaq enzyme, etc. used herein were purchased from Takara Biotech Dalian Co., Ltd.); the PCR reaction conditions were as follows: \textcircled{1} 94° C. 4 min, \textcircled{2} 94° C. 30 s, \textcircled{3} 58° C. 30 s, \textcircled{4} 72° C. 1 min, \textcircled{5} 33 cycles from \textcircled{2} to \textcircled{4}, \textcircled{6} 72° C. 7 min, \textcircled{7} 25° C. storage. According to the sequencing results, a homozygous knockout mutant was obtained in this experiment, and denoted as ZmGS3^{ko}, and there was a 34 bp deletion at the target site (FIG. 17). The mutant gene sequence of ZmGS3^{ko} was shown in SEQ ID NO: 31, and the putative amino acid sequence of the mutant gene sequence was shown in SEQ ID NO: 32.

3.7 Identification of Alkali Tolerance of Rice Plants OsGS3^{ko} and Maize Plants ZmGS3^{ko}

The wild-type rice ZH11 and OsGS3^{ko}, the wild-type maize KN5585 and ZmGS3^{ko} were treated with 75 mM mixed alkali solution. The phenotypic observation results showed that under normal water treatment, there was no significant difference in the survival rates between rice OsGS3^{ko} and ZH11, and there was no significant difference between the maize ZmGS3 deletion mutant line ZmGS3^{ko} and the wild-type KN5585. However, under the treatment of 75 mM alkali solution, the survival rate of the rice OsGS3^{ko} transgenic plants was significantly increased compared with ZH11 (FIG. 12E-F). Similarly, the survival rate of the maize ZmGS3^{ko} transgenic plants was significantly increased compared with KN5585 (FIG. 12H-I). The results showed that the GS3 genes of both rice and maize were important genes that negatively regulated saline-alkali tolerance, and this function was conserved in Gramineae plants maize and rice.

These mutant genes or mutant proteins could be transferred to other rice or maize or other hybrid materials through conventional cross-pollination to cultivate new alkali-tolerant lines.

### Example 4. Similar Effects of AT1 Homologous Gene in Wheat

Common wheat (Triticum aestivum) is a hexaploid plant with genomes A, B and D. The homologous gene of AT1 in wheat is named TAGS or TaAT1 (the two terms can be used interchangeably), with three copies: TaGS-4A gene, TaGS-7A gene and TaGS-7D gene, which are three homologous genes in wheat, and the genomic sequences are shown in SEQ ID NO: 59, SEQ ID NO: 60 and SEQ ID NO: 61, respectively. TaGS protein comprises any one or more of TaGS-4A1 protein, TaGS-4A2 protein, TaGS-7A protein or TaGS-7D protein. Among them, TaGS-4A1 protein and TaGS-4A2 protein are two transcripts of wheat TaGS-4A gene, and their amino acid sequences are SEQ ID NO: 55 and SEQ ID NO: 56 respectively. TaGS-7A gene expresses TaGS-7A protein as shown in SEQ ID NO: 57. TaGS-7D) gene expresses TaGS-7D protein as shown in SEQ ID NO: 58. The following experiments demonstrated that by adjusting any one or more of the above-mentioned multiple nucleic acid molecules, the expression amount of the corresponding TaGS protein could be changed, thereby achieving the regulation of saline-alkali tolerance in wheat plants, which could be applied to plant breeding.

### 4.1 Selection of Wheat TaGS Target Site and Construction of Knockout Vector

### 1) Selection of Wheat TaGS Target Site

There are three homologous genes TaGS-4A, TaGS-7A and TaGS-7D in wheat, and the corresponding gene ID numbers are TraesCS4A02G474000, TraesCS7A02G017700 and TraesCS7D02G015000, respectively. A suitable target site was selected, a conservative target sequence was used to target the three homologous genes, a knockout vector was constructed, and the selected target site was located in the first exon (FIG. 18).

One of the chains in the double-stranded target knocked out by CRISPR technology had the following structure: 5-Nx-NGG-3, wherein N in PAM (NGG) represented any one of A, T, C and G, N in Nx represented any one of A, T, C and G, and x=20. In this example, the target sequence of the selected TaGS gene was as follows, and the underlined bases were PAM.

TaGS gene target sequence:
(SEQ ID NO: 70)
AAGTCCCCGCTCGACCCCTGCGG

After the knockout vector was transformed into wheat, the Cas9 protein cut in the target sequence region under the guidance of sgRNA, thereby forming a DNA double-strand break, triggering the in vivo spontaneous repair mechanism. In the process of spontaneously repairing the gap in the cells, mutations (herein, “mutations” referred to mutations in a broad sense, including insertion, deletion, mutation in a narrow sense, etc., most of which are gene function inactivating mutations) would be introduced to the cells.

### 2) Construction of Recombinant Vector

(1) pBUE411 (addgen #62200) plasmid was digested with restriction endonuclease BsaI, and the vector backbone of about 12.5 kb was recovered and named BUE411.

(2) According to the designed TaGS gene target sequence (SEQ ID NO: 70), the following primers with sticky ends (underlined part) were synthesized:

TaGS-1F:
(SEQ ID NO: 71)
GGCGAAGTCCCCGCTCGACCCCTG.
TaGS-1R:
(SEQ ID NO: 72)
AAACCAGGGGTCGAGCGGGGACTT.

(3) TaGS-1F and TaGS-1R were annealed to form a double-stranded DNA with sticky ends, named TaGS-1, which was ligated to the gel recovered product BUE411 in step 1 to obtain a recombinant plasmid pBUE411-TaGS-1. The structure of the recombinant plasmid pBUE411-TaGS-1 was described as follows: the small fragment between the recognition sequences of the two restriction endonucleases BsaI of the pBUE411 plasmid was replaced with the DNA fragment as shown in positions 1-20 of SEQ ID NO: 70 to obtain the recombinant plasmid.

The experimental methods used in the examples of the present application were all conventional methods unless otherwise specified.

The materials, reagents, etc. used in the examples of the present application, unless otherwise specified, could all be obtained from commercial channels.

### 4.2 Transformation of Wheat

Wheat immature embryo calli were genetically transformed using the Agrobacterium EHA105 carrying the recombinant plasmid pBUE411-TaGS-1 constructed in Example 1. After transformation, complete regenerated plants (i.e., TO generation) were obtained through tissue culture.

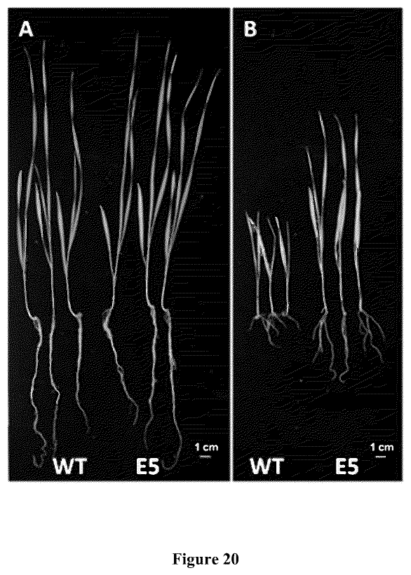

After subculturing in the T2 generation, the transgenic plant E5 with functional loss of three homologous genes of TaGS gene: TaGS-4A, TaGS-7A, and TaGS-7D, was obtained. It was revealed by Sanger sequencing that the TaGS gene TaGS-4A of the E5 plant was a homozygous mutation, and both TaGS-7A and TaGS-7D were biallelic mutations (as shown in FIG. 19). The TaGS-4A mutation of the E5 plant was a 13 bp deletion of CCCGCTCGACCCC bases (SEQ ID NO: 73) at positions 6-18; the first mutation of TaGS-7A of the E5 plant was a 1 bp deletion of base C at position 18, and the second mutation was a 2 bp deletion of bases CC at positions 17-18; the first mutation of TaGS-7D of the E5 plant was a 1 bp deletion of base G at position 20, and the second mutation was a 2 bp deletion of bases TG at positions 19-20.

### 4.3 Saline-Alkali Tolerance Phenotype of Wheat TaGS Gene Triple-Mutation Mutant Under Salt-Alkaline Stress

First, the T2 generation TaGS gene triple-mutation mutant seeds E5 obtained in Example 4.2 and the wild-type Fielder were sown in a soil matrix of vermiculite and nutrient soil mixed in a ratio of 1:1, 12 seeds were sown per hole, and 2 replicates were set. Next, the soil was irrigated with 75 mM mixed alkali solution (pH 9.2, NaHCO₃:Na₂CO₃ at a molar ratio of 5:1) until saturated as treatment group, and irrigated with clean water as control group, the trays were placed on a flat ground for uniform absorption, and three replicates were set for each treatment group. After sufficient absorption, the plug trays were placed in a model plant glass greenhouse, and clean water was replenished later. The seedling growth environment was as follows: the light/dark time was 16 h/8 h, the day/night temperature range was 28/26° C., and the relative humidity was 60% to 70%.

The results showed that after treatment with saline-alkaline stress (75 mM mixed alkali solution), the E5 mutant line exhibited significantly higher saline-alkali tolerance than the wild-type, significantly higher aboveground biomass than the wild-type, and significantly weaker degree of root growth inhibition in underground part than the wild-type (as shown in FIG. 20).

Similar to the above manipulation, 100 mM or 125 mM mixed alkaline solution (pH 9.2, molar ratio of 5:1 NaHCO₃:Na₂CO₃) was used for irrigation until the soil was saturated in treatment group, and clean water was used in the control group. The E5 mutant line exhibited significantly higher saline-alkali tolerance than the wild-type, and weaker degree of chlorosis and wilting than the wild-type.

From the above results, it could be seen that the following technical effects were achieved in the above example of the present invention: by gene editing the nucleic acid molecules used for transcription and translation of TaGS protein in wheat, the expression amount of TaGS protein in wheat was reduced, the saline-alkali tolerance of wheat was improved, and new wheat plants with improved saline-alkali tolerance were obtained.

### 4.4 Other Detection of Wheat TaGS Gene Triple-Mutation Mutant

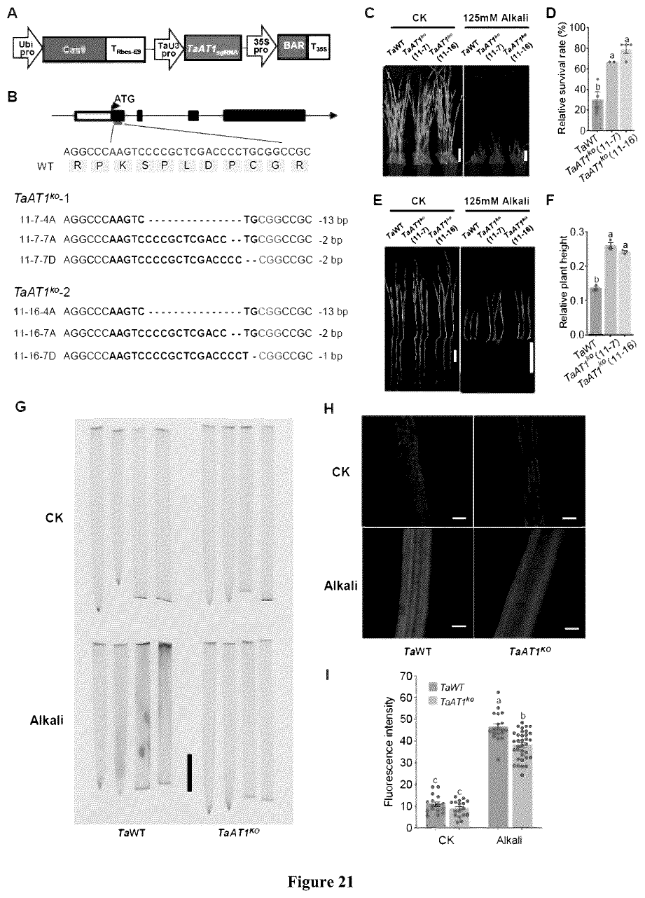

We used all three copies of TaAT1 (also known as TaGS) gene to generate TaAT1 null mutant (i.e., TaGS gene triple-mutation mutant). Phenotypic analysis showed that under 125 mM (104.2 mM NaHCO₃ and 20.8 mM Na₂CO₃, pH 9.7-9.8) mixed alkali treatment, the survival rates of TaAT1^{ko}-1 and TaAT1^{ko}-2 were 122% and 164% higher than that of TaWT, respectively (FIGS. 21C, D, E, and F). It could be seen that knocking out all copies of TaAT1 could greatly enhance the saline-alkali tolerance of wheat.

To further investigate whether TaAT1 adopts a conservative mechanism to resist salt-alkaline stress like sorghum and rice, DAB (3,3-diaminobenzidine) and H₂DCFDA staining method was used to determine the ROS accumulation in TaAT1^{ko} line. It could be seen from FIGS. 21G and H that under salt-alkali treatment, the intracellular H₂O₂ level of TaAT1^{ko} line was significantly lower than that of TaWT, which was consistent with the AT1^{ko} phenotypes of other species. The results showed that, like the role of AT1 gene in sorghum, maize and rice, TaAT1 gene was an important gene that negatively regulated the saline-alkali tolerance of wheat. Genetic modification of TaAT1 gene could also effectively improve the growth performance of wheat in saline-alkali soil, and the mechanism was conservative.

### Example 5. Improvement of Crop Yield in High-Sodium Soil

To evaluate the usefulness of AT1/GS3 gene in crop production, we conducted field trials on sorghum, rice, maize and millet in high-sodium soil containing natural alkali. These sorghum, rice, maize and millet plants had different natural alleles and genetically modified AT1/GS3 genes. The fields were located in two areas of saline-alkali regions in China: Da'an area of Jilin Province (northern China) and Pinglou area of Ningxia (northwest China). These two areas are major crop producing areas in China, but crop yields are limited due to the presence of large areas of saline-alkali land.

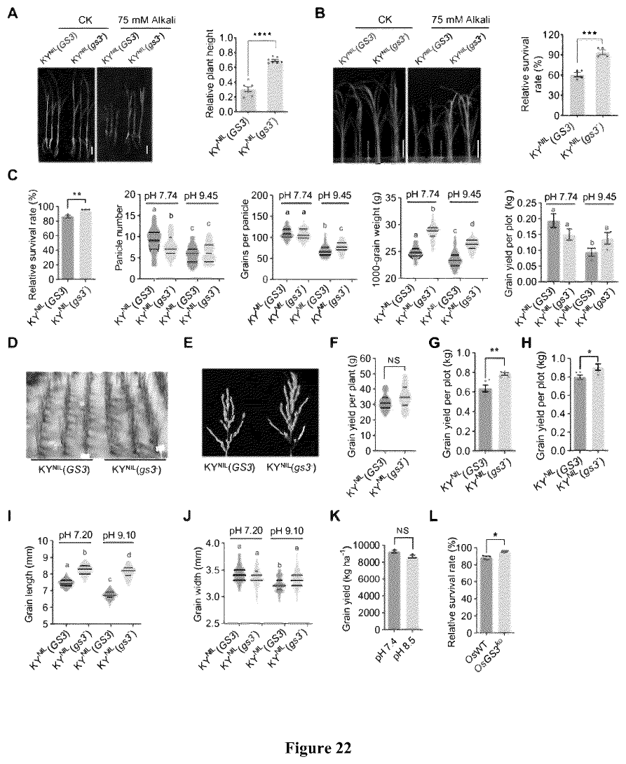

KY^{NIL} (GS3) is an elite rice variety Kongyu131 carrying OsGS3-2 (with an in-frame 3-bp insertion at the C-terminal relative to OsGS3-1), which is functionally equivalent to OsGS3-1. KY^{NIL} (gs3⁻) has an introgressed OsGS3-3, a complete loss-of-function allele, in the Kongyu131 background. Before the field trial, KY^{NIL} (GS3) and KY^{NIL} (gs3⁻) were tested for alkali tolerance. The test was conducted in a greenhouse at the seedling stage with a mixed alkali concentration of 75 mM. As expected, KY^{NIL} (gs3⁻) showed higher alkali tolerance than Kongyu131 (FIGS. 22A and B).

We then conducted an experiment to compare NIL in two different soils at pH 9.45 and 7.74 (control) using a mix of high-sodium soil and nutrient soil from the same region. Under sodium soil with a pH of 9.45, KY^{NIL} (gs3⁻) significantly outperformed KY^{NIL} (GS3) in terms of relative survival rate, number of grains per panicle, grain weight, and yield (FIG. 22C), except for the number of panicles per plant (FIG. 22C).

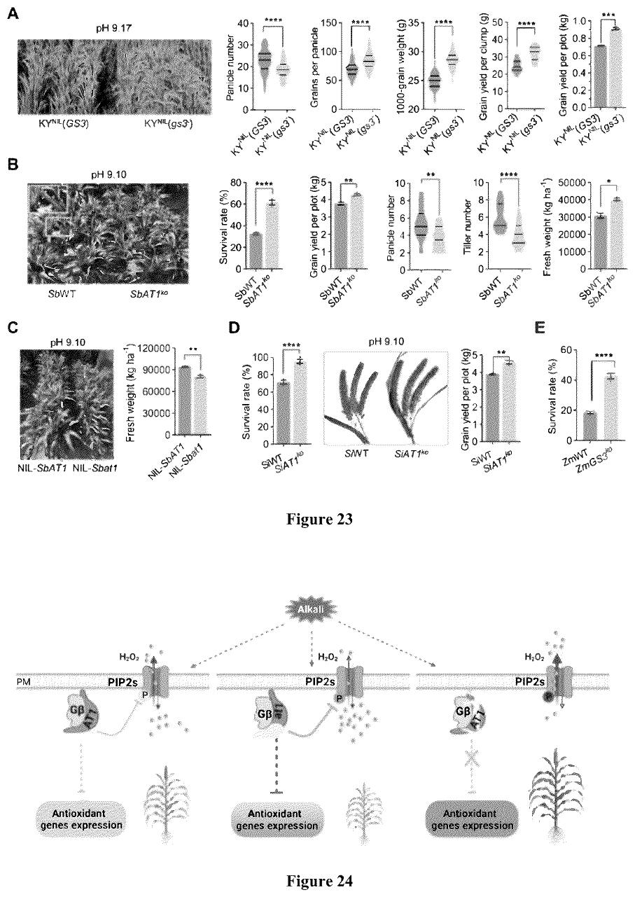

We also conducted field trials in Da'an, Jilin Province, China, where the soil pH was 9.17. KY^{NIL} (gs3⁻) performed much better than KY^{NIL} (GS3) at both the seedling stage (FIG. 22D) and harvest stage (FIG. 23A, left panel), indicating the enhanced alkali tolerance of KY^{NIL} (gs3⁻). At harvest stage, KY^{NIL} (gs3⁻) rice produced larger panicles (FIG. 22E), a higher number of grains per panicle (FIG. 23A, third panel), and increased grain weight (FIG. 23A, fourth panel), resulting in a 29.3% increase in grain yield per clump (FIG. 23A, fifth panel), and that the grain yield of KY^{NIL} (gs3⁻) rice was 27.8% higher than that of the control (FIG. 23A, right panel). In the same plantings in the field with a pH of 5.58, the difference in yield per plant between KY^{NIL} (gs3⁻) and the control was only 10.3% (FIG. 22F). In the second year, the grain yield of NIL in sodic and acidic soils was studied at the same location. The yield increased by 22.4% in the alkaline soil and 6.64% in the control field (FIGS. 22G and H), similar to the results observed previously. In addition, knockout of OsGS3 contributed to grain length in both sodic and neutral soils. We also found that knockout of OsGS3 contributed to grain width in sodic soils but not in neutral soils (Figure S22I and J). These results suggested that a non-functional allele of GS3 could achieve higher crop yields in high-sodium soils.

Due to its superior quality and high yield, the improved elite rice Zhongkefa5 (ZKF5) has been planted in more than 100,000 hectares in northern China since 2018. ZKF5 has a non-functional allele of GS3 (OsGS3-3), similar to KY^{NIL} (gs3⁻). The field performance of ZKF5 was then tested in relatively high-sodium soils (pH 8.5-8.7) and low-sodium soils (pH 7.4-7.6) in the summer of 2021. At the end of the growing season, the field production data from more than 30 hectares of land were obtained from the local farmers' association. We found that the yield in the sodium-containing fields (high sodium) were only reduced by 7.8% compared with that of the neutral fields (low sodium) (FIG. 22K). These large-scale field production data also suggested that the use of the GS3-3 allele (i.e., non-functional allele) in rice production in sodium-containing lands could allow the cultivation of rice with improved crop yields.

In addition, we also planted Zhonghua 11 (ZH11) rice and its OsGS3 knockout line OsGS3^{ko} in the same soils with two pH values in a greenhouse, in which the control had pH 7.74 and the alkaline soil had pH 9.45. Although we were unable to harvest seeds of ZH11 due to its photoperiod sensitivity when grown in Jilin Province, China, the higher relative survival rate observed in the OsGS3^{ko} line (FIG. 22L) indicated that it was more tolerant to alkaline conditions.

Sorghum, maize, and millet were tested in the Pingluo area of Gansu Province, China. The Pingluo area is located in northwestern China and consists of arid land with high sodium soils. In this area, pH naturally rises during the growing season due to changes in groundwater levels (B. P. Singh, A. L. Cowie, K. Y. and Chan, Soil health and climate change. New York: Springer-Verlag Berlin Heidelberg 29, (2011). doi:10.1007/978-3-642-20256-8). Wheatland wild-type and SbAT1^{ko} sorghum planted in the same plot had a pH of 8.97 in spring and an flowering pH of 9.27 in August. The SbAT1^{ko} line had a survival rate of over 60%, while the Wheatland wild-type had a survival rate of only 33% (FIG. 23B, first and second panels). Leaf burn, a symptom that typically occurs in monocot crops affected by high salt or high sodium stress, was observed in most Wheatland wild-type plants but not in plants of the SbAT1^{ko} line (FIG. 23B, first panel). At the harvest stage, the numbers of tillers and panicles of the SbAT1^{ko} line (FIG. 23B, fourth and fifth panels) were lower than those of the control plant, but the yield was 20.1% higher than that of the control plant (FIG. 23B, third panel). Since the whole plant of sorghum is usually used for silage, we measured the fresh weight of whole plant biomass and found that the plant fresh weight of the SbAT1^{ko} line was 30.5% higher than that of the control plant (FIG. 23B, right panel). The grain yield and whole plant biomass of the SbAT1^{ko} line were higher than those of the wild type, indicating that the field performance of the SbAT1^{ko} line sorghum in sodium-containing land (saline-alkali land) was better. In the same area, we also planted NIL-SbAT1 and NIL-SbAT1 lines in summer. At the end of August, we found that NIL-SbAT1 was superior to NIL-Sbat1 (FIG. 23C), and the difference was similar to the results recorded in the greenhouse experiment (FIG. 6B).

Millet SiAT1^{KO} and its wild-type control Ci846 were also planted with sorghum in the same area. The survival rate of the SiAT1^{KO} line at the seedling stage was close to 100%, while the survival rate of the wild-type Ci846 was only about 75% (FIG. 23D, left panel). In addition, at the harvest stage, the panicle and kernel sizes of the knockout line SiAT1^{KO} were also larger than those of the control plant (FIG. 23D, middle panel), and the yield of SiAT1^{KO} was about 19.5% higher than that of the control plant (FIG. 23D, right panel).

On the same plot of land, we also planted the maize ZmGS3 knockout line and its wild-type control line KN5585 (ZmWT). At the seedling stage, one month after planting, the relative survival rate of the ZmGS3^{ko} line was about 42.5%, while the relative survival rate of the wild-type control was only about 18.5% (FIG. 23E). After 3 months of growth, most individuals of the KN5585 line died, while 7.4% of the individuals of the knockout line survived. Although none of these maize plants matured to produce grains due to the inherent sensitivity of maize to alkaline conditions, the ZmGS3 knockout line showed enhanced alkali tolerance.

In summary, we were able to conclude that non-functional mutations in AT1 homologous genes, whether obtained from natural variation or generated by gene editing, can improve field performance of crops in terms of biomass or yield when grown in sodic soils (saline-alkali land).

### Example 6. Study of Action Mechanism of AT1

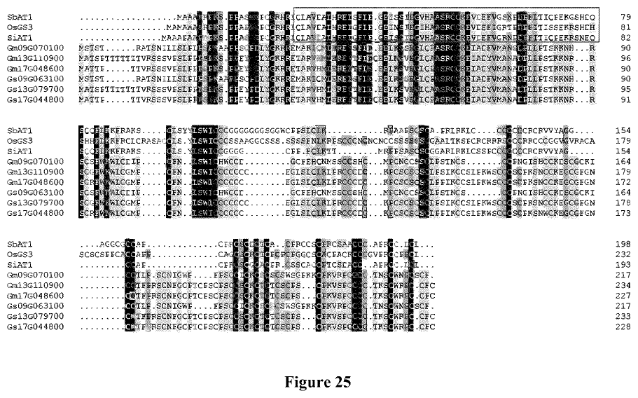

Based on our studies, we designed a model of putative Gγ subunit AT1-mediated alkaline stress response in plants (FIG. 24). Under alkaline stress, PIP2s (PIP2 water channel protein) functions as a H₂O₂ export protein. The Gγ subunit AT1 may pair with GB to negatively regulate the phosphorylation of PIP2s, thereby reducing the H₂O₂ export capacity of PIP2s, resulting in excessive accumulation of H₂O₂ and causing sensitivity of plants to alkaline stress. The truncated form of AT1, i.e., at1, further inhibits H₂O₂ export activity and leads to high sensitivity of plants to alkaline stress. However, the natural non-functional form of AT1 or the knockout of AT1 homologous gene releases the inhibitory effect on PIP2s and effectively improves the tolerance against alkaline stress in crops.

### Example 7. Similar Functions of AT1 Soybean Homologous Genes

Through BLAST, three genes with relatively high homology to sorghum AT1 protein sequence were retrieved in wild soybean (Glycine soja) and cultivated soybean (Glycine max) (both were tetraploid) (FIG. 25 and Table 2, the similarity was about 40% to about 50%), and all of them contained Gγ-like domain. However, all three phylogenetic analysis diagrams showed that they were distantly related to sorghum, rice, maize, millet and wheat.

TABLE 2
Alignment of full-length amino acid sequences with sorghum
AT1 (using the sequence alignment tool of VectorBuilder)

| | Species | Identity % | Similarity % |
|---|---|---|---|
| Alignment of full- | Rice | 58.40 | 61.76 |
| length amino acid | Millet | 76.64 | 79.44 |
| sequence with | Maize | 83.73 | 84.21 |
| sorghum AT1 | Wheat-4A transcript 1 | 41.67 | 46.08 |
| | Wheat-4A transcript 2 | 55.07 | 61.84 |
| | Wheat-7A | 55.07 | 62.32 |
| | Wheat-7D | 55.56 | 62.8 |
| | Soybean-09 | 35.8 | 46.91 |
| | Soybean-13 | 38.4 | 46.01 |
| | Soybean-17 | 38.91 | 47.08 |
| | Wild soybean-09 | 35.8 | 46.91 |
| | Wild soybean-13 | 38.55 | 46.18 |
| | Wild soybean-17 | 38.52 | 47.08 |

TABLE 3
Alignment of GGL domain with sorghum AT1 GGL domain
(using the sequence alignment tool of VectorBuilder)

| | Species | Identity % | Similarity % |
|---|---|---|---|
| Alignment of GGL | Rice | 79.10 | 86.57 |
| domain with sorghum | Millet | 89.55 | 95.52 |
| AT1 GGL domain | Maize | 94.03 | 95.52 |
| | Wheat-4A transcript 1 | 76.12 | 85.07 |
| | Wheat-4A transcript 2 | 76.12 | 85.07 |
| | Wheat-7A | 74.63 | 85.07 |
| | Wheat-7D | 77.61 | 86.57 |
| | Soybean-09 | 47.22 | 62.5 |
| | Soybean-13 | 44.44 | 61.11 |
| | Soybean-17 | 43.66 | 61.97 |
| | Wild soybean-09 | 47.22 | 62.5 |
| | Wild soybean-13 | 45.07 | 61.97 |
| | Wild soybean-17 | 43.06 | 61.11 |

In Examples 1-5, we have demonstrated that the AT1/GS3 gene played a conserved and important role in the salinity-alkaline stress response of five monocotyledonous cereals (i.e., sorghum, millet, rice, maize and wheat). Although further studies are needed to determine whether genetic modification of AT1/GS3 genes can also regulate saline-alkali tolerance in dicotyledonous plants (such as soybean), considering that the AT1 homologous gene in soybean has a certain degree of identity with the AT1 gene in sorghum, especially the high identity of the GGL domain (about 40% to 50%), our preliminary experimental results (the data were not shown) support that the AT1 homologous gene in soybean will play a similar conservative role in regulating saline-alkali tolerance as the homologous genes in sorghum, millet, rice, maize and wheat. For example, reducing the expression of all copies of AT1 homologous genes in soybean, especially knocking out the GGL domain, or using all copies of non-functional alleles, could improve the saline-alkali tolerance of soybean; conversely, if the expression of AT1 homologous genes in soybean was increased (e.g., overexpressed), or the C-terminal truncation mutant of AT1 homologous gene was expressed, soybeans with saline-alkali sensitivity could also be obtained.

The sequences of AT1 homologous genes in soybean are shown in Table 4.

TABLE 4
Sequences of AT1 homologous genes in soybean

Cultivated soybean Glyma.13G110900
>G.max Wm82.a4.v1|Glyma.13G110900.1 CDS (SEQ ID NO: 74)
ATGGCCACTTCTCCCACCACCACCACCACCACCACCGTTCGTTCTTCTTCCGTCCCTTCTCTGCCTC
CACCCTCCCCCAAGTCCCCGCCGGAGTACCCAGATTTGTATGGCAAGCGCCGTGAAACCGCCAGG
GTTCACATGCTCGAGAGGGAAATCACTTTTCTCGAGCAGGAAGAATTAAAATCTGTTGAAGGCCT
TCAAGCAGCTTCAAGATGCTGCAAAGAGATTGCTGATTATGTGATGGCAAACGCAGATCCTCTGT
TACCTTCAACCAAGAAGAACCGTCGGTCATGTGGCTTCTGGAAGTGGCTCTGTGGCATGCCCTGTT
TTAACCTCTCTTGGATCTGTTGCTGCTGTTGCTGCTGTGAAGGGTTATCTTTACAACTAAAATTGCC
ACGCTGCTGTTGTGACTGCAAACCATGCAGTTGCAGTTGCAGCTGTCTTCCATCCATCAAATGCTG
CTCCTTACCAAAGTGGAGCTGTTGTTGCTCTTGCCCCAAATCAAATTGCTGTAAAGAGGGTTGCGG
TTTTGGAAATTGTTGTACTTTCCCACGTAGTTGCAATTTTGGGTGCCCAACTTGTCCATCTTGCCCC
TCTTGCTGCAGTTGCAAATGCACCTGCACTTGCTCTTGCCCAAGCTGTCCAAAGGTAAGCCCATGT
TGCTGTTGTACAAAGTCATGTTGGAGACCTTGTTGTTTTTGTTGCTAG
>G.max Wm82.a4.v1|Glyma.13G110900.1.p (235AA) (41-105, similar to GGL domain) (SEQ ID
NO: 75)
MATSPTTTTTTTVRSSSVPSLPPPSPKSPPEYPDLYGKRRETARVHMLEREITFLEQEELKSVEGLQAAS
RCCKEIADYVMANADPLLPSTKKNRRSCGFWKWLCGMPCFNLSWICCCCCCCEGLSLQLKLPRCCCD
CKPCSCSCSCLPSIKCCSLPKWSCCCSCPKSNCCKEGCGFGNCCTFPRSCNFGCPTCPSCPSCCSCKCTC
TCSCPSCPKVSPCCCCTKSCWRPCCFCC*
Cultivated soybean Glyma.17G048600
>G.max Wm82.a4.v1|Glyma.17G048600.1 CDS (SEQ ID NO: 76)
ATGGCCACTACTCCCACCACCGTTCGTTCTTCTTCCGTCCCTTCTCTGCCTCCACCCTCTCCTAAGT
CCCCGCCGGAGTACCCAGATTTGTATGGCAAGCGCCGTGAAACCGCCAGGGTTCACATGCTCGAG
AGGGAAATCACTTTTCTCGAGGAAGAATTAAAATCTGTTGAGGGCCTTCAACCAGCTTCAAGATG
CTGCAAAGAGATTGCTGATTATGTGATGGCAAACGCAGATCCCCTGTTACCTTCAACCAAGAAGA
ACCGCCGGTCATGTCGCTTCTGGAAGTGGCTCTGTGGCATGCCCTGTTTTAACCTCTCTTGGATCT
GCTGTTGTTGCTGCTGTGAAGGGTTATCTTTACAACTAAAATTGCCACGCTGCTGTTGTGACTGCA
AACCATGCAGTTGCAGTTGCAGTTGTCTTCCACCCATCAAATGCTGCTCCTTACCAAAGTGGAGCT
GTTGTTGCTCTTGCCCCAAATCAAATTGCTGTAAAGAGGGTTGTGGTTTTGGAAATTGTTGTACTT
TCCCACGTAGTTGCAATTTTGGGTGTCCAACTTGTCCATCTTGCCCCTCTTGCTGCAGTTGCAAATG
CACCTGCACTTGCTCTTGCCCAAGCTGTCCGAAGGTAAGCCCATGTTGCTGTTGTACAAAGTCATG
CTGGAGACCATGTTGTTTTTGTTGCTAG
>G.max Wm82.a4.v1|Glyma.17G048600.1.p (228AA) (36-99, similar to GGL domain) (SEQ ID
NO: 77)
MATTPTTVRSSSVPSLPPPSPKSPPEYPDLYGKRRETARVHMLEREITFLEEELKSVEGLQPASRCCKEI
ADYVMANADPLLPSTKKNRRSCRFWKWLCGMPCFNLSWICCCCCCEGLSLQLKLPRCCCDCKPCSCS
CSCLPPIKCCSLPKWSCCCSCPKSNCCKEGCGFGNCCTFPRSCNFGCPTCPSCPSCCSCKCTCTCSCPSC
PKVSPCCCCTKSCWRPCCFCC*
Cultivated soybean Glyma.09G070100
>G.max Wm82.a4.v1|Glyma.09G070100.1 CDS (SEQ ID NO: 78)
ATGAGCACTTCCACAAGGGCAACTTCCAACATCCTTCTCTCTCTGCCACTGCCCTCTCCTAAGGCG
GCGCCTCCTTCGTGCCCAGATTTGTATGGAAAGCGTCGCGAAATGGCCAAGATTCAGATGCTGGA
AAGAGAGATAAGTTTCCTCGAGGAAGAGTTAAAATCTTCTGAAGGCCTTCAACCAGCTTCAAGAT
GCTGCAAAGAGATTGCCGATTTTGTGATGGCAAACTCAGATCCTCTGTTACCTACGAGCAAGAAG
AACCACCGGTCATGTAGCTTCTGGAAGTGGCTGTGTGACATCCCTTGCTTTAACTTGTCTTGGATC
TGCCATTGGTGCTGTGATGGGTGCTTTGAACATCAAAACATGTCAAGTTGTTGTTCCCACTGCATG
CCATGCAATTGCTGTTCTAGTTGTCTTCCATCTACCAATTGCTCTTGCTGCCCTAATGGAATATCAC
ATTGCTGCAAAGATAGTTGTGGTTGCAAAATTTGTTGCACTCTCCCAAGTTGCAATATTGGGTGGC
CTTTTCCCTCTTGCTGCATTTGCAAATGCTCTTGCTCTTGCTCTTGGTCAGGCCCAAAAAAATGTCC
CAAGGTTCGTCCATGTTGCTGTTGTACAAATTCCTGTTGGAACCCCTGTTCATGTTTCTAG
>G.max Wm82.a4.v1|Glyma.09G070100.1.p (217AA) (36-99, similar to GGL domain) (SEQ ID
NO: 79)
MSTSTRATSNILLSLPLPSPKAAPPSCPDLYGKRREMAKIQMLEREISFLEEELKSSEGLQPASRCCKEIA
DFVMANSDPLLPTSKKNHRSCSFWKWLCDIPCFNLSWICHWCCDGCFEHQNMSSCCSHCMPCNCCSS
CLPSTNCSCCPNGISHCCKDSCGCKICCTLPSCNIGWPFPSCCICKCSCSCSWSGPKKCPKVRPCCCCTN
SCWNPCSCF*
Wild soybean GlysoPI483463.17G044800
>G.soja v1.1|GlysoPI483463.17G044800.1 CDS (SEQ ID NO: 80)
ATGGCCACTACTCCCACCACCGTTCGTTCTTCTTCCGTCCCTTCTCTGCCTCCACCCTCTCCTAAGT
CCCCGCCGGAGTACCCAGATTTGTATGGCAAGCGCCGTGAAACCGCCAGGGTTCACATGCTCGAG
AGGGAAATCACTTTTCTCGAGCAGGAAGAATTAAAATCTGTTGAGGGCCTTCAACCAGCTTCAAG
ATGCTGCAAAGAGATTGCTGATTATGTGATGGCAAACGCAGATCCCCTGTTACCTTCAACCAAGA
AGAACCGCCGGTCATGTCGCTTCTGGAAGTGGCTCTGTGGCATGCCCTGTTTTAACCTCTCTTGGA
TCTGCTGTTGTTGCTGCTGTGAAGGGTTATCTTTACAACTAAAATTGCCACGCTGCTGTTGTGACT
GCAAACCATGCAGTTGCAGTTGCAGTTGTCTTCCACCCATCAAATGCTGCTCCTTACCAAAGTGGA
GCTGTTGTTGCTCTTGCCCCAAATCAAATTGGTGTAAAGAGGGTTGTGGTTTTGGAAATTGTTGTA
CTTTCCCACGTAGTTGCAATTTTGGGTGTCCAACTTGTCCATCTTGCCCCTCTTGCTGCAGTTGCAA
ATGCACCTGCACTTGCTCTTGCCCAAGCTGTCCGAAGGTAAGCCCATGTTGCTGTTGTACAAAGTC
ATGCTGGAGACCATGTTGTTTTTGTTGCTAG
>G.soja v1.1|GlysoPI483463.17G044800.1.p (229AA) (36-100, similar to GGL domain) (SEQ
ID NO: 81)
MATTPTTVRSSSVPSLPPPSPKSPPEYPDLYGKRRETARVHMLEREITFLEQEELKSVEGLQPASRCCKE
IADYVMANADPLLPSTKKNRRSCRFWKWLCGMPCFNLSWICCCCCCEGLSLQLKLPRCCCDCKPCSC
SCSCLPPIKCCSLPKWSCCCSCPKSNWCKEGCGFGNCCTFPRSCNFGCPTCPSCPSCCSCKCTCTCSCPS
CPKVSPCCCCTKSCWRPCCFCC*
Wild soybean GlysoPI483463.13G079700
>G.soja v1.1|GlysoPI483463.13G079700.1 CDS (SEQ ID NO: 82)
ATGGCCACTTCTCCCACCACCACCACCACCACCACCGTTCGTTCTTCTTCCGTCCCTTCTCTGCCTC
CACCCTCCCCCAAGTCCCCGCCGGAGTACCCAGATTTGTATGGCAAGCGCCGTGAAACCGCCAGG
GTTCACATGCTCGAGAGGGAAATCACTTTTCTCGAGGAAGAATTAAAATCTGTTGAAGGCCTTCA
AGCAGCTTCAAGATGCTGCAAAGAGATTGCTGATTATGTGATGGCAAACGCAGATCCTCTGTTAC
CTTCAACCAAGAAGAACCGTCGGTCATGTGGCTTCTGGAAGTGGCTCTGTGGCATGCCCTGTTTTA
ACCTCTCTTGGATCTGTTGCTGCTGTTGCTGCTGTGAAGGGTTATCTTTACAACTAAAATTGCCAC
GCTGCTGTTGTGACTGCAAACCATGCAGTTGCAGTTGCAGCTGTCTTCCATCCATCAAATGCTGCT
CCTTACCAAAGTGGAGCTGTTGTTGCTCTTGCCCCAAATCAAATTGCTGTAAAGAGGGTTGCGGTT
TTGGAAATTGTTGTACTTTCCCACGTAGTTGCAATTTTGGGTGCCCAACTTGTCCATCTTGCCCCTC
TTGCTGCAGTTGCAAATGCACCTGCACTTGCTCTTGCCCAAGCTGTCCAAAGGTAAGCCCATGTTG
CTGTTGTACAAAGTCATGTTGGAGACCTTGTTGTTTTTGTTGCTAG
>G.soja v1.1|GlysoPI483463.13G079700.1.p (234AA) (41-104, similar to GGL domain) (SEQ
ID NO: 83)
MATSPTTTTTTTVRSSSVPSLPPPSPKSPPEYPDLYGKRRETARVHMLEREITFLEEELKSVEGLQAASR
CCKEIADYVMANADPLLPSTKKNRRSCGFWKWLCGMPCFNLSWICCCCCCCEGLSLQLKLPRCCCDC
KPCSCSCSCLPSIKCCSLPKWSCCCSCPKSNCCKEGCGFGNCCTFPRSCNFGCPTCPSCPSCCSCKCTCT
CSCPSCPKVSPCCCCTKSCWRPCCFCC*
GlysoPI483463.09G063100
>G.soja v1.1|GlysoPI483463.09G063100.1 CDS (SEQ ID NO: 84)
ATGAGCACTTCCACAAGGGCAACTTCCAACATCCTTCTCTCTCTGCCACTGCCCTCTCCTAAGGCG
GCGCCTCCTTCGTGCCCAGATTTGTATGGAAAGCGTCGCGAAATGGCCAAGATTCAGATGCTGGA
AAGAGAGATAAGTTTCCTCGAGGAAGAGTTAAAATCTTCTGAAGGCCTTCAACCAGCTTCAAGAT
GCTGCAAAGAGATTGCCGATTTTGTGATGGCAAACTCAGATCCTCTGTTACCTACGAGCAAGAAG
AACCACCGGTCATGTAGCTTCTGGAAGTGGCTGTGTGACATCCCTTGCTTTAACTTGTCTTGGATC
TGCCATTGGTGCTGTGATGGGTGCTTTGAACATCAAAACATGTCAAGTTGTTGTTCCCACTGCATG
CCATGCAATTGCTGTTCTAGTTGTCTTCCATCTACCAATTGCTCTTGCTGCCCTAATGGAATATCAC
ATTGCTGCAAAGATAGTTGTGGTTGCAAAATTTGTTGCACTCTCCCAAGTTGCAATATTGGGTGGC
CTTTTCCCTCTTGCTGCATTTGCAAATGCTCTTGCTCTTGCTCTTGGTCAGGCCCAAAAAAATGTCC
CAAGGTTCGTCCATGTTGCTGTTGTACAAATTCCTGTTGGAACCCCTGTTCATGTTTCTAG
>G.soja v1.1|GlysoPI483463.09G063100.1.p (217AA) (36-99, similar to GGL domain) (SEQ
ID NO: 85)
MSTSTRATSNILLSLPLPSPKAAPPSCPDLYGKRREMAKIQMLEREISFLEEELKSSEGLQPASRCCKEIA
DFVMANSDPLLPTSKKNHRSCSFWKWLCDIPCFNLSWICHWCCDGCFEHQNMSSCCSHCMPCNCCSS
CLPSTNCSCCPNGISHCCKDSCGCKICCTLPSCNIGWPFPSCCICKCSCSCSWSGPKKCPKVRPCCCCTN
SCWNPCSCF*

TABLE 5
Amino acid sequence of GGL domain

Sorghum AbAT1 GGL domain (SEQ ID NO: 15, corresponding to the amino acid fragment at
positions 22-88 of SEQ ID NO: 4)
LQLAVDALHREISFLEGEISSIEGVHAASRCCKEVDEFVGSNPDPFLTIQPEKGSHDQSQQFLKKFR
Millet SiAT1 GGL domain (SEQ ID NO: 86, corresponding to the amino acid fragment at positions
25-91 of SEQ ID NO: 8)
LQLAVDALHREIGFLEGEISSIDGVHAASRCCKEVDEFVGRNPDPFITIQPEKRSNEQSQQFLKKFR
Rice OsGS GGL domain (SEQ ID NO: 87, corresponding to the amino acid fragment at positions
24-90 of SEQ ID NO: 16)
LQLAVDALHREIGFLEGEINSIEGIHAASRCCREVDEFIGRTPDPFITISSEKRSHDHSHHFLKKFR
Maize ZmGS GGL domain (SEQ ID NO: 88, corresponding to the amino acid fragment at positions
24-90 of SEQ ID NO: 18)
LQLAVDALHREIGFLEGEISSIEGVHAASRCCKEVDEFVGRNPDPFLTIQQERGSHDQSQQFLKKFR
Wheat TaAT1-4A1 GGL domain (SEQ ID NO: 89, corresponding to the amino acid fragment at
positions 18-84 of SEQ ID NO: 55)
LQLAVDALHRQISFLEGEISSIEGLHAASICCKEVDEFIGKNADPFITISSEKGNADQSHRSPKKIR
Wheat TaAT1-4A2 GGL domain (SEQ ID NO: 89, corresponding to the amino acid fragment at
positions 18-84 of SEQ ID NO: 56)
LQLAVDALHRQISFLEGEISSIEGLHAASICCKEVDEFIGKNADPFITISSEKGNADQSHRSPKKIR
Wheat TaAT1-7A GGL domain (SEQ ID NO: 90, corresponding to the amino acid fragment at
positions 18-84 of SEQ ID NO: 57)
LQLAVDALHRQISFLEGEINSIEGLHAASICCKEVDEFIGKNADPFITISSEKGNAEQSHPFPKKIR
Wheat TaAT1-7D GGL domain (SEQ ID NO: 91, corresponding to the amino acid fragment at
positions 18-84 of SEQ ID NO: 58)
LQLAVDALHRQISFLEGEISSIEGLHAASICCKEVDEFIGKNADPFITISSEKGNADQSHRFPKKIR
Cultivated soybean Gm09G070100 GGL domain (SEQ ID NO: 92, corresponding to the amino acid
fragment at positions 36-99 of SEQ ID NO: 79)
EMAKIQMLEREISFLEEELKSSEGLQPASRCCKEIADFVMANSDPLLPTSKKNHRSCSFWKWLC
Cultivated soybean Gm13G110900 GGL domain (SEQ ID NO: 93, corresponding to the amino acid
fragment at positions 41-105 of SEQ ID NO: 75)
ETARVHMLEREITFLEQEELKSVEGLQAASRCCKEIADYVMANADPLLPSTKKNRRSCGFWKWLC
Cultivated soybean Gm17G048600 GGL domain (SEQ ID NO: 94, corresponding to the amino acid
fragment at positions 36-99 of SEQ ID NO: 77)
ETARVHMLEREITFLEEELKSVEGLQPASRCCKEIADYVMANADPLLPSTKKNRRSCRFWKWLC
Wild soybean Gs09G063100 GGL domain (SEQ ID NO: 92, corresponding to the amino acid fragment
at positions 36-99 of SEQ ID NO: 85)
EMAKIQMLEREISFLEEELKSSEGLQPASRCCKEIADFVMANSDPLLPTSKKNHRSCSFWKWLC
Wild soybean Gs13G079700 GGL domain (SEQ ID NO: 95, corresponding to the amino acid fragment
at positions 41-104 of SEQ ID NO: 83)
ETARVHMLEREITFLEEELKSVEGLQAASRCCKEIADYVMANADPLLPSTKKNRRSCGFWKWLC
Wild soybean Gs17G044800 GGL domain (SEQ ID NO: 96, corresponding to the amino acid fragment
at positions 36-100 of SEQ ID NO: 81)
ETARVHMLEREITFLEQEELKSVEGLQPASRCCKEIADYVMANADPLLPSTKKNRRSCRFWKWLC
Note:
(i) The amino acid sequences of the GGL domains of wheat TaAT1-4A1 and TaAT1-4A2 are identical (SEQ ID NO: 89);
(ii) The amino acid sequences of the GGL domains of cultivated soybean Gm09G070100 and wild soybean Gs09G063100 are identical (SEQ ID NO: 92 ).

Those skilled in the art will further appreciate that the present invention may be implemented in other specific forms without departing from its spirit or central features. Since the foregoing description of the present invention discloses only exemplary embodiments thereof, it should be understood that, other variations are considered to be within the scope of the present invention. Therefore, the present invention is not limited to the specific embodiments described in details herein. Instead, reference should be made to the appended claims to indicate the scope and content of the present invention.

1. An isolated nucleic acid molecule, encoding a protein comprising an amino acid sequence having at least 40% identity or having at least about 60% similarity to SEQ ID NO: 15, wherein the nucleic acid molecule is used to regulate saline-alkali tolerance in a plant, or for producing a plant with saline-alkali tolerance or salt-alkali sensitivity.

2. The nucleic acid molecule according to claim 1, wherein the nucleic acid molecule encodes a protein comprising an amino acid sequence as shown in any one of SEQ ID NOs: 15 and 86-96.

3. The nucleic acid molecule according to claim 1, which encodes an amino acid sequence selected from:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85.

4. A mutant protein, which is encoded by a variant nucleic acid molecule generated from a frameshift mutation in a nucleic acid molecule encoding one of the following amino acid sequences:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85;
wherein the mutant protein has reduced activity or no activity as compared with the protein encoded by the nucleic acid molecule before the frameshift mutation.

5. (canceled)

6. An expression cassette or recombinant vector, which comprises the nucleic acid molecule according to claim 1.

7. (canceled)

8. A cell, which comprises the nucleic acid molecule according to claim 1.

9. (canceled)

10. A method for producing a saline-alkali-tolerant plant, comprising:
in a plant, reducing the expression level of all alleles encoding a protein comprising an amino acid sequence having at least 40% identity, or having at least about 60% similarity to SEQ ID NO: 15, or making the all alleles non-expressed,
wherein the growth of the saline-alkali-tolerant plant under saline-alkali conditions is better than that of a wild-type plant thereof,
wherein the saline-alkali conditions comprise growth conditions at pH >7.5 and Na⁺ concentration >75 mM or growth conditions at pH >8.0 and Na⁺ concentration >50 mM.

11. The method according to claim 10, wherein reducing the expression level of the all alleles or making the all alleles non-expressed in the plant is performed by gene editing, targeted mutagenesis, chemical induction, radiation induction, natural mutation, RNAi or adding a substance capable of inhibiting the expression of a target gene.

12. (canceled)

13. (canceled)

14. The method according to claim 10, wherein a GGL domain or GGL-like domain, or the first exon portion at N-terminal of the all alleles is knocked out or mutated.

15. (canceled)

16. (canceled)

17. The method according to claim 10, which further comprises identifying an offspring plant of one or more generations obtained by selfing a parent plant comprising a knockout or mutation of the all alleles or comprising non-functional allele(s), or by crossing said parent plant with another parent plant comprising a knockout or mutation of the all alleles or comprising non-functional allele(s),
wherein the knockout or mutation of the all alleles results in that the protein encoded by each of the all alleles has a reduced activity or no activity.

18. The method according to claim 10, wherein a GGL domain or GGL-like domain, or the first exon portion at N-terminal, of all alleles of a gene encoding an amino acid sequence selected from the following amino acid sequences is knocked out or mutated:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85.

19. A plant or plant material, wherein all alleles encoding a protein comprising an amino acid sequence having at least 40% identity, or having at least about 60% similarity, to SEQ ID NO: 15 are knocked out or mutated;
wherein the knockout or mutation in the all alleles results in that the protein encoded by each of the all alleles has a reduced activity or no activity.

20. (canceled)

21. The plant or plant material according to claim 19, wherein a GGL domain or GGL-like domain, or the first exon portion at N-terminal of all alleles of the gene encoding the amino acid sequence selected from the following amino acid sequences is knocked out or mutated:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85.

22. (canceled)

23. A method for producing seeds of a hybrid plant, comprising:
(i) hybridizing a first parent plant with a second parent plant, wherein in the first parent plant and the second parent plant, all alleles encoding a protein comprising an amino acid sequence having at least 40% identity, or having at least about 60% similarity, to SEQ ID NO: 15 are knocked out or mutated; and
(ii) harvesting seeds of the hybrid plant or progeny thereof.

24. A method for producing seeds of a conventional breeding plant, comprising:
propagating a parental seed to harvest an offspring seed thereof, wherein in the parental seed, all alleles encoding a protein comprising an amino acid sequence having at least 40% identity, or having at least about 60% similarity to SEQ ID NO: 15 have been knocked out or mutated, so that the protein is not expressed or has an reduced expression level as compared with a wild-type control plant.

25. The method according to claim 23, wherein in the first parent plant and the second parent plant, all alleles encoding a protein comprising one of the following amino acid sequences have been knocked out or mutated:
(i) an amino acid sequence having at least 80%, 85%, 90%, 95%, 99% or 100% identity and similar or identical function to SEQ ID NO: 4;
(ii) an amino acid sequence having at least 80%, 85%, 90%, 95%, 99% or 100% identity and similar or identical function to SEQ ID NO: 8;
(iii) an amino acid sequence having at least 80%, 85%, 90%, 95%, 99% or 100% identity and similar or identical function to any one of SEQ ID NOs: 16-17;
(iv) an amino acid sequence having at least 80%, 85%, 90%, 95%, 99% or 100% identity and similar or identical function to SEQ ID NO: 18;
(v) an amino acid sequence having at least 80%, 85%, 90%, 95%, 99% or 100% identity and similar or identical function to any one of SEQ ID NOs: 55-58; or
(vi) an amino acid sequence having at least 80%, 85%, 90%, 95%, 99% or 100% identity and similar or identical function to any one of SEQ ID NOs: 75, 77, 79, 81, 83 or 85.

26. The method according to claim 23, wherein the first parent plant and/or the second parent plant is an inbred line plant.

27. (canceled)

28. A method for producing a saline-alkali sensitive plant, comprising:
increasing the expression level of a gene encoding a protein comprising an amino acid sequence having at least 40% identity, or having at least about 60% similarity to SEQ ID NO: 15 in a plant, or expressing or overexpressing a C-terminal truncated protein encoded by the gene mutant in a plant.

29. The method according to claim 28, wherein the expression level of target gene is increased by introducing into the plant an exogenous nucleic acid molecule encoding an amino acid sequence having at least 40% identity, or having at least about 60% similarity to SEQ ID NO: 15.

30. The method according to claim 29, wherein a genetic material carrying the nucleic acid molecule is introduced into a cell or tissue of the plant, and the genetic material exists in the plant in the form of free or integrated into a chromosome of the plant, and then the cell or tissue with the genetic material introduced is cultured into a whole plant to obtain the saline-alkali sensitive plant.
